(19) United States
(12) Patent Application Publication
(10) Pub. No.: US 2020/0390743 A1
(43) Pub. Date: Dec. 17, 2020

(54) Methods and Compositions For Improving Outcomes of Cancer Patients

(71) Applicant: Reven IP HoldCo LLC, Golden, CO (US)

(72) Inventors: James Ervin, Novi, MI (US); Hendrik J. Van Wyk, Glendale, CO (US); Brian D. Denomme, Northville, MI (US); Mariette L. Van Wyk, Glendale, CO (US); Peter Pacult, Denver, CO (US); Michael A. Volk, Broomfield, CO (US); Natalie M. Pizzimenti, Washington, MI (US)

(21) Appl. No.: 16/899,699

(22) Filed: Jun. 12, 2020

Related U.S. Application Data
(60) Provisional application No. 62/860,642, filed on Jun. 12, 2019.

(51) Int. Cl. A61K 31/375; A61K 31/51; A61K 33/06; A61K 31/714; A61K 31/455; A61K 31/4415; A61K 31/675; A61K 31/16; A61K 33/00; A61P 3/10; A61P 3/12; A61P 7/00; A61P 3/14

(52) U.S. Cl. CPC A61K 31/375; A61K 31/51; A61K 33/06; A61K 31/714; A61K 31/455; A61K 31/4415; A61P 3/14; A61K 31/16; A61K 33/00; A61P 3/10; A61P 3/12; A61P 7/00; A61K 31/675

(57) ABSTRACT

This disclosure provides compositions and methods for improving outcomes in cancer patients.

### CROSS-REFERENCE TO RELATED APPLICATION

The application claims the benefit of priority of U.S. Provisional Application No. 62/860,642 filed Jun. 12, 2019, the entire disclosure of which is incorporated herein by reference in its entirety.

### FIELD OF THE INVENTION

The present invention relates generally to methods for improving outcomes of cancer patients by administration of compositions as disclosed herein.

### BACKGROUND OF THE INVENTION

Homeostasis is the ability of an organism to maintain a condition of equilibrium or stability within its internal environment, particularly when faced with external changes. Some examples of homeostatically-controlled systems in humans include the regulation of a constant body temperature, blood glucose levels, and extracellular ionic species concentrations. Acid-base homeostasis relates to the proper balance of acids and bases in extracellular fluids, i.e., the pH of the extracellular fluid. In humans, the pH of plasma is approximately 7.4 and is tightly maintained around that value by three interconnected control systems: (1) buffering agents, including bicarbonate, phosphate, and proteins; (2) the respiratory system, which impacts the partial pressure of carbon dioxide in blood plasma; and (3) the renal system, which excretes waste acids and bases. Acid homeostasis is also influenced by metabolic load, which serves as a primary source of acid in the body. For instance, a high glucose diet can increase total acid burden from metabolic sources, consequently placing a bigger burden on acid homeostasis control mechanisms.

Inefficiencies in these control systems and factors, which increase acid, such as from metabolic sources, may gradually result in unstable internal environments that increase the risk of illness or exacerbate existing conditions. These inefficiencies may be caused by natural aging processes or may be self-inflicted through various lifestyle choices. For example, many pathological roots in oncology trace to failings along several key points of chemistry. Deficiencies in oxygen delivery impair aerobic metabolism and fuel the vascularization of tumors.

It would be beneficial to develop methods to restore the imbalances caused by inefficient, ineffective, or over-stressed homeostatic processes to achieve better outcomes for patients having a condition, such as cancer. The presently disclosed subject matter addresses, in whole or in part, these and other needs in the art.

### SUMMARY OF THE INVENTION

This disclosure addresses the need mentioned above in a number of aspects. In one aspect, this disclosure provides a method for preventing, alleviating, or treating a hypoxia-related disease or condition, comprising administering an effective amount of a composition to a subject in need thereof to improve oxygen transport and thereby elevate blood oxygen levels, wherein the composition comprises: at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In some embodiments, the hypoxia-related disease or condition is cancer, angiogenesis, or an angiogenesis-related disorder. In some embodiments, the cancer is a tumor or a solid tumor. Cancer can be any one of breast cancer, pancreatic cancer, ovarian cancer, colon cancer, lung cancer, non-small cell lung cancer, in situ carcinoma (ISC), squamous cell carcinoma (SCC), thyroid cancer, cervical cancer, uterine cancer, prostate cancer, testicular cancer, brain cancer, bladder cancer, stomach cancer, hepatoma, melanoma, glioma, retinoblastoma, mesothelioma, myeloma, lymphoma, and leukemia.

In some embodiments, the composition increases intracellular HCO3⁻ level and thereby promotes hemoglobin affinity for oxygen.

In some embodiments, the subject suffers a blood electrolyte imbalance, which is a result of excess acid or bicarbonate.

In some embodiments, the method comprises elevating pO₂ level in the venous blood in the subject.

In another aspect, this disclosure also provides a method for treating a subject suffering from a condition characterized by elevated serum calcium. The method comprises administering an effective amount of a composition to the subject to reduce blood calcium levels, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for restoring tumor suppressor protein p53 function in a subject. The method comprises administering an effective amount of a composition to the subject, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for suppressing tumor aggression in a subject having a cancer while restoring angiogenesis in healthy tissue of the subject. The method comprises administering an effective amount of a composition to the subject to increase eNOS and suppress iNOS, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for treating a subject having a cancer and suffering from elevated blood glucose related to the cancer. The method comprises administering an effective amount of a composition to the subject to improve pituitary, thyroid and renal function, thereby reducing blood glucose levels, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In some embodiments, the composition reduces cortisol levels, thereby reducing circulating glucose by relieving mitochondrial stress and endoplasmic reticulum stress.

In another aspect, this disclosure also provides a method for inhibiting poly ADP ribose polymerase (PARP). The method comprises administering to a subject in need thereof an effective amount of a composition, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for restoring a disturbed bone marrow microenvironment. The method comprises administering an effective amount of a composition to a subject in need thereof, the method comprising at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In yet another aspect, this disclosure also provides a method for promoting apoptosis in cancer. The method comprises administering an effective amount of a composition to a subject in need thereof, thereby eliciting a temporarily elevated acidic pH in the bloodstream to further decreasing intracellular pH which results in acidic stress and apoptosis in cancer cells, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In some embodiments, the subject is a human or a veterinary subject. The composition can be delivered by intravenous, intramuscular, or parenteral administration, oral administration, otic administration, topical administration, inhalation administration, transmucosal administration, and transdermal administration. In some embodiments, the intravenous administration is a bolus delivery. In some embodiments, the composition is administered by local delivery.

In some embodiments, the methods described above further comprise administering to the subject a second agent. The composition can be administered to the subject before or after administrating the second agent. In some embodiments, the composition is administered concurrently with the second agent.

In some embodiments, the second agent is an anti-cancer agent. In some embodiments, the composition is administered after the subject is treated with adjuvant or neoadjuvant chemotherapy. In some embodiments, the composition is administered between 1 and 90 days after the subject is treated with adjuvant or neoadjuvant chemotherapy.

In some embodiments, the methods described above further comprise administering to the subject a second dose of the composition. The second dose can be administered to the subject between 1 and 30 days after a first dose is administered.

In some embodiments, the pharmaceutical grade acid is a physiologically acceptable acid (e.g., hydrochloric acid, ascorbic acid, acetic acid, or a combination thereof).

In some embodiments, the pH buffering agent is a physiologically acceptable buffer (e.g., sodium bicarbonate, a phosphate buffer, sodium hydroxide, an organic acid, an organic amine, ammonia, a citrate buffer, a synthetic buffer creating specific alkaline conditions, or a combination thereof). In some embodiments, the synthetic buffer is tris-hydroxymethyl aminomethane.

In some embodiments, the composition further comprises one or more ingredients selected from the group consisting of vitamins, salts, acids, amino acids or salts thereof, and stabilized oxidative species.

In some embodiments, the composition further comprises ascorbic acid. In some embodiments, the composition further comprises dehydroascorbic acid.

In some embodiments, the composition further comprises other recognized antioxidant defense compounds including nonenzymatic compounds such as tocopherol (aTCP), coenzyme Q10 (Q), cytochrome c (C) and glutathione (GSH) and enzymatic components including manganese superoxide dismutase (MnSOD), catalase (Cat), glutathione peroxidase (GPX), phospholipid hydroperoxide glutathione peroxidase (PGPX), glutathione reductase (GR); peroxiredoxins (PRX3/5), glutaredoxin (GRX2), thioredoxin (TRX2) and thioredoxin reductase (TRXR2).

In some embodiments, the composition further comprises one or more of a sodium salt, a magnesium salt, a potassium salt, and a calcium salt.

In some embodiments, the composition further comprises one or more of a B vitamin, vitamin C, and vitamin K.

In some embodiments, the composition is formulated for intravenous, bolus, dermal, oral, otic, suppository, buccal, ocular, or inhalation delivery. The composition can be formulated in hypotonic, isotonic, or hypertonic form.

In some embodiments, the composition comprises pharmaceutical grade of:

900±90 mg of L-Ascorbic Acid;

63.33±6.33 mg Thiamine HCl;

808±80.8 mg of Magnesium Sulfate;

1.93±0.193 mg of Cyanocobalamin;

119±11.9 mg of Niacinamide;

119±11.9 mg of Pyridoxine HCl;

2.53±0.253 mg of Riboflavin 5′Phosphate;

2.93±0.293 mg of Calcium D-Pantothenate;

840±84 mg of Sodium Bicarbonate;

4.5±0.45 mM of HCl; and

water in an amount to obtain a final composition volume of 20 mL.

In another aspect, the composition is provided in a kit comprising (a) a first vial containing a stable therapeutic composition comprising a buffer solution comprising at least one pharmaceutical grade acid and at least one pharmaceutical grade pH buffering agent, wherein the buffer solution is sufficient to reduce the physiological bloodstream pH of a subject by 0.1 to 1.1, and wherein the buffer solution has a buffer capacity sufficient to sustain the reduction of the physiological bloodstream pH of the subject for between 1 minute and 1 week; and optionally (b) instructions for use.

In yet another aspect, the composition is provided in a kit comprising (a) a first vial containing an intravenous buffer solution comprising at least one pharmaceutical grade acid in a sterile aqueous solution; (b) a second vial containing at least one pharmaceutical grade pH buffering agent in a sterile aqueous solution, wherein, when combined, the contents of the two vials form an intravenous buffer solution, wherein the concentration of the pharmaceutical grade acid and the pharmaceutical grade pH buffering agent in the buffer solution is sufficient to provide a total titratable acid content of from 60 mmol/L to 3000 mmol/L when administered to a subject, and wherein the selection of the pharmaceutical grade acid and the pharmaceutical grade pH buffering agent is effective to provide a buffer solution pH of between 4 and 7.7; and optionally (c) instructions for use.

In some embodiments, the composition further comprises 100±10 mg of dehydroascorbic acid. In some embodiments, the buffer solution is sufficient to reduce the physiological bloodstream pH of a subject between about 0.01 and about 1.1. In some embodiments, the buffer solution has a buffer capacity sufficient to sustain the reduction of the physiological bloodstream pH of the subject for between 1 minute and 1 week.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

In the following, a brief description of the appended figures will be given. The figures are intended to illustrate the present invention in more detail. However, they are not intended to limit the subject matter of the invention in any way.

FIG. 1 is a diagram showing the typical chemiosmotic gradient of hydrogen ions between the inner-membrane and matrix in a normally functioning mitochondria in a mammalian cell.

FIG. 2 is a diagram showing the reduced chemiosmotic gradient of hydrogen ions in mitochondria in a mammalian cell with a dysfunctional metabolism, as may occur after a prolonged exposure to a poor diet or lack of exercise.

FIG. 3 is a diagram showing the chemiosmotic flow of ions into and out of the cell of a subject having a hypoxic crisis or as observed in phases of acid-base disturbance, such as during or following exercise, or as observed during or following use of the composition of the invention.

FIG. 4 is a diagram showing the chemiosmotic flow of ions into and out of the cell of a subject having had the hypoxic crisis corrected by use of the composition of the invention.

FIG. 5 is a diagram showing the amplitude and duration of an acid state shift caused by different formulations of compositions of the present disclosure.

FIG. 6 shows clinical (non-GCP)/non-clinical (GCP) efficacy of the compositions as described: Perfusion, Plaque, Healing

FIG. 7 shows pH and HCO₃− response on Day 0, Day 2, and Day 82. Monitoring occurred for 60 minutes, starting with the infusion occurring at time 0. The infusion completed at 45 minutes, and the recovery was monitored for 15 minutes.

FIG. 8 shows Ca²⁺ and K⁺ response on Day 0, Day 2, and Day 82. Monitoring occurred for 60 minutes, starting with the infusion occurring at time 0. The infusion completed at 45 minutes, and the recovery was monitored for 15 minutes.

FIG. 9 shows blood glucose response on Day 0, Day 2, and Day 82. Monitoring occurred for 60 minutes, starting with the infusion occurring at time 0. The infusion completed at 45 minutes, and the recovery was monitored for 15 minutes.

FIG. 10 shows sO2, pO2, pCO2 response on Day 0, Day 2, and Day 82. Monitoring occurred for 60 minutes, starting with the infusion occurring at time 0. The infusion completed at 45 minutes, and the recovery was monitored for 15 minutes.

FIG. 11 shows the recovery of RBC.

FIGS. 12A and 12B (collectively “FIG. 12”) show vasodilation response as observed over 10 dosing events. FIG. 12A shows the changes in the plasma volume over the course of 10 treatments. FIG. 12B shows hematocrit as a measure of vasodilation over ten treatments. 100% represents post-treatment plasma volume as estimated from hematocrit concentration.

FIG. 13 shows blood pressure and heart rate response similar to post-exercise adaptation. Data from Feb. 18, 2019—dose 26—day 82.

FIG. 14 shows photos (with permission) of a post-chemotherapy patient before and after treatment with the inventive composition. This is a 69-year-old male during chemotherapy (photo on the left) on Jul. 26, 2018, prior to any treatment with the composition as described. The photo on the right occurred on Mar. 8, 2019 after receiving 29 treatments of the composition as described.

FIG. 15 shows pH and HCO₃⁻ response of acid shifting composition (Dose 1, Day 1).

FIG. 16 shows sO₂, pCO₂, pO₂ response of acid shifting composition (Dose 1, Day 1).

FIG. 17 shows pH and HCO₃⁻ response of acid shifting composition with vitamins and minerals (Dose 4, Day 6).

FIG. 18 shows sO₂, pCO₂, pO₂ response of acid shifting composition with vitamins and minerals (Dose 4, Day 6).

FIG. 19 shows pH and HCO₃⁻ response of acid shifting composition with vitamins and minerals (Dose 5, Day 8).

FIG. 20 shows sO₂, pCO₂, pO₂ response of acid shifting composition with vitamins and minerals (Dose 5, Day 8) of Subject 2, after administration of the therapeutic composition.

FIG. 21 shows venous pH, HCO₃⁻, and sO₂ response after 4 doses in all 3 subjects.

FIG. 22 shows Fibrinogen and platelet response in all 3 horses follow 4 treatments of the inventive composition (also referred to as RJX G2).

FIG. 23 shows Ca²⁺ and K⁺ response after 4 doses in all 3 subjects.

FIG. 24 shows ACTH (surrogate for Cortisol), T4, glucose, and insulin response after 4 doses of the composition as described in horses (upper right: literature reference in horse section 12 relating human T4 and basal metabolic rate/calories).

FIG. 25 shows white blood cell (wbc), eosinophil, and lyme antibody response after 4 doses in all 3 horses.

FIG. 26 shows lyme surface protein response after 4 treatments in 3 horses.

### DETAILED DESCRIPTION OF THE INVENTION

The present disclosure is based on, at least in part, the unexpected discovery that reducing physiological bloodstream pH in a subject is useful in treating, ameliorating, and preventing many conditions and diseases and symptoms thereof in a subject in need. The invention provides a stable therapeutic composition that can be administered to a subject in need thereof, in order to provide the requisite shift in blood pH.

### I. Overview

Many pathological roots in oncology trace to failings along several key points of chemistry. For one, deficiencies in oxygen delivery impair aerobic metabolism and fuel the vascularization of tumors. As a second, glycolysis-centric metabolism produces steady acidification within a neighborhood of cells and can lead to broad acidosis, with many enzyme function, sensory, and metabolic consequences. Third, intracellular and bloodstream calcium commonly elevates within cancer cells and host-healthy cells while depleting in compartments like the endoplasmic reticulum, to affect sensory function, cell division, mutation, and other functions. Fourth, impairments in the metabolic chain as promoted by acidification, hypoxia, calcium signaling, and gut impairments from these factors, increase reactive oxygen species (ROS), which promotes cell destruction and affects white blood cell phenotype and response signaling. Thus oxygen status, acid-base status, calcium status, and ROS are fundamental forces that have broad influence in cancer. As described below, these collective influences can distort glucose utility, disturb the bone marrow microenvironment “niche,” promote elevations in poly (ADP-ribose) polymerase (PARP) to promote mutagenicity, promote angiogenesis to vascularize tumors, enable acid-base adaptations in cancer to enhance their proliferation, impair p53 to over-ride proper apoptotic controls and impair overall metabolism to drive sensory disturbance along with fatigue and cognitive impairment.

These roots are explored below in more detail:

1. Enhancements in oxygen delivery impair cancer by empowering otherwise healthy cells to survive and empower select cells to help fight cancer:
a. Claim elements
   i. Enhanced oxygen helps healthy cells better survive cancer.
   ii. Enhanced oxygen helps white blood cells (WBC's) fight cancer
   iii. Tumor hypoxia is relevant for tumor growth, metabolism, resistance to chemotherapy, and metastasis. Those with hypoxic tumors have a worse prognosis, and potentially have a more aggressive phenotype (Vaupel).
   iv. Reducing hypoxic presentation, reduces hypoxia-inducible factor (HIF), to reduce expression and activity of glycolytic enzymes which impairs cancer metabolism
b. How the composition as described accomplishes this:
   i. Correct impaired hemoglobin affinity for Oxygen: H+ to drive HCO3− (bicarbonate) to intracellular and alkaline pH upon rebound
   ii. Vasodilation: Ca²⁺↓+Mg²⁺↑: endothelin constriction ↓, Ca²⁺↓ yields endothelial nitric oxide synthase (eNOS)↓+pH stimulus: (nitric oxide (NO) binds to its receptor soluble guanylyl cyclase (sGC)) NO-sGC dilation ↑, B3: prostacyclin dilation ↑
   iii. Stimulate red blood cell (RBC) and platelet recovery: partial pressure of oxygen (pO2) stimulus to produce erythropoietin (EPO) and alkaline bone marrow
   iv. Mitigate hyper-coagulation: alkaline conditions with resolution of Ca²⁺ and glucose signaling
   v. Reverse vascular plaque to open vessels: alkaline+low ROS+eNOS correction to reverse plaque progression
   vi. Restore SELECTIVE angiogenesis through: reducing cytosolic Ca²⁺ and complete metabolic chain with less ROS, which allows eNOS to translocate from Golgi to cell membrane to enable endothelium-directed vessel growth and sustain vascular health and perfusion in healthy tissue (B Vitamins+increased Antioxidants (Anti-Ox) via Ascorbic acid and dehydroascorbic acid (DHAA) stimulated glutathione reduce ROS), increasing intracellular bicarbonate and restoring more alkaline conditions □ local hypoxia or other inducible moments (pH shift) allow better distinguishing from the background in order to reliably trigger NO.

2. The composition as described corrects Ca²⁺ presentation and signaling. Most cancer patients have a disruption in their regulation of calcium, eventually progressing to hypercalcemia. With continued worsening of hypercalcemia, patients can experience polyuria, abdominal pains, and other weaknesses (Rosner, et al. Advances in Chronic Kidney Disease, vol. 21, no. 1, Elsevier Ltd, 2014, pp. 7-17).
a. Claim elements
   i. Resolving hypercalcemia and reducing cytosolic Ca+2 while increasing Ca+2 in the endoplasmic reticulum.
   ii. Reducing elevated pain response and impaired sensory function that is commonly coincident with cancer.
b. How the composition as described accomplishes this:
   i. Magnesium reduces cytosolic calcium both actively and/or passively. Actively magnesium has been shown to reduce osteogenic transcription factors that can be expressed in vascular smooth muscle cells. Passively it is able to do this through binding in the blood to inorganic phosphate. The main source of calcified plaques is calcium phosphate bonds, therefore through the binding of magnesium to phosphate there is a reduction in the availability for calcium to bind and form plaques. (Ter Braake et al.)
   ii. Reductions in metabolic H+ lead to reductions in cytosolic calcium by reducing the exchange of H+ from the cell with Ca+2 from the bloodstream
   iii. Adenosine tri-phosphate (ATP) excess allows sarcoendoplasmic reticulum ATPase (SERCA) to pump cytosolic Ca+2 into Endoplasmic Reticulum stores.

3. Metabolic dysfunction towards glycolysis is a major component of cancer metabolism. The composition as described reverses metabolic dysfunction by completing metabolic chain to increase ATP while reducing metabolic acid H+, Lactate, ROS. The alteration in the metabolism in cancer cells progress to alter the physiology of the cancer patient in order for it to thrive. Alterations in the more alkaline intracellular pH of the cell promote glycolysis (the breakdown of glucose to lactic acid) and suppress gluconeogenesis. Also, lactate dehydrogenase, which is responsible for the conversion of pyruvate to lactate, functions optimally around pH 7.5, which is similar to what the intracellular pH is of a cancer cell (Webb, et al. Nature Reviews Cancer, vol. 11, no. 9, 2011, pp. 671-77). ROS are generated as a byproduct of the electron transport chain. Since cancer cells favor glycolysis this results in an abnormal ratio of NADH/NAD+ and increased leakage of electrons causing a heightened generation of ROS.
a. How the composition as described accomplishes this:
   i. Reduction in cytosolic calcium restores hydrogen control of the chemiosmotic gradient to restore oxidative phosphorylation (OxPhos) (Poburko D, Demaurex N. Pflugers Arch. 2012; 464(1):19-26; Celsi F, et al. Biochim Biophys Acta. 2009; 1787(5):335-344.)
   ii. Enhancements in ascorbic acid, glutathione and redox status improve the efficiency of the Krebs cycle and/or electron transport chain (ETC) activity. (Quijano C, et al. Redox Biol. 2016; 8:28-42.)
   iii. Enhanced oxygen increases metabolic yield (Nakazawa M S, et al. Nat Rev Cancer. 2016; 16(10):663-673.)
   iv. Enhanced B-vitamins increase metabolic yield (Parikh S, et al. Curr Treat Options Neurol. 2009; 11(6):414-430.)
   v. Mg²⁺ in the composition as described promote more complete Krebs cycle and facilitates Mg-ATP and Mg-ADP (adenosine di-phosphate) cycling to increase metabolic yield (Yamanaka R, et al. Sci Rep. 2016; 6:30027.)
   vi. More complete metabolic chain makes more ATP
   vii. More complete metabolic chain makes less acid H+(Burns J S, Manda G. Int J Mol Sci. 2017; 18(12):2755.)
   viii. More complete metabolic chain reduces Lactate/Oxygen debt and enables faster re-payment of past debt (Burns J S, Manda G. Int J Mol Sci. 2017; 18(12):2755.)

4) Another attribute of cancer cells is that they generate additional ROS (Shi X, et al. Antioxid Redox Signal. 2012; 16(11):1215-1228). The observed ROS increase in cancer cells may result from the activation of oncogenes, inactivation of tumor suppressor genes, high metabolism, and mitochondrial dysfunction (Trachootham D., et al., Nat Rev Drug Discov. 2009; 8:579-591). In addition, cancer can steadily drive acidosis and metabolic chain impairments in otherwise healthy cells to increase their ROS production. Increased ROS can challenge antioxidant systems and disrupt the redox balance in favor of oxidative stress, which can damage tissues and promote pro-inflammatory signaling. Such disturbances in ROS contribute to cell survival, proliferation, and metastasis in a variety of cancers.

Thus targeting these disruptions in the redox balance could be an important target in improving cancer outcomes and preventing recurrence.

An incomplete metabolic chain is one source of elevated ROS in tissues. Metabolic chain completion is commonly recognized to be supported by oxygen delivery, B-vitamins, magnesium, antioxidants, and reduced intracellular calcium so that reactions in the Krebs cycle, electron chain transport (ETC) and Oxidative Phosphorylation (OxPhos) promote more oxygen reactions to CO₂ and H₂O and less to O⁻, H₂O₂, OCl⁻, HOCl, and other oxidant forms. (Griffiths H R, et al. Redox Biol. 2017; 12:50-57.). Thus, reducing metabolic sources of ROS is one way to reduce antioxidant consumption and promote more redox balance.

Antioxidants provide a second force in the redox balance as they convert oxidative species like O—, H2O2, OCl—, HOCl into neutralized forms like H₂O or intermediate forms that other antioxidants can reduce to neutral forms (Rahal A, et al. Biomed Res Int. 2014; 2014:761264.).

One means to increase antioxidants is supplementation, such as administered intravenously. For instance, ascorbic acid can be supplemented to enhance redox balance (Bouamama S, et al. Appl Physiol Nutr Metab. 2017; 42(6):579-587.). In addition, dehydroascorbic acid (DHAA) is a reduced form of ascorbic acid that can also be supplemented.

DHAA is another component of the antioxidant maintenance system. It has a unique property as a form that is readily absorbed through glucose receptors, so as to be especially accessible to the eyes and brain where it can be cycled to ascorbic acid by action of glutathione to increase antioxidant reserves. As another nuance of DHAA, works as a stimulant in the liver among other sites to promote Glutathione production. Here pyridoxine works with sulfate chemistries to make glutathione, which enhances recycling of DHAA among other redox roles.

Catalase is an antioxidant that is known to target peroxide in the peroxisomes that has also been shown to be sensitive to acid-base and calcium status. It has been found that the pH status within peroxisomes is largely determined by that of the cytosol. In addition, it has been found that the calcium status in the peroxisome is elevated when the endoplasmic reticulum is in a condition of stress. Under acidic conditions with elevated calcium, it has been found that catalase levels are reduced in the peroxisome along with impairments in peroxisome functions such as fatty acid metabolism and myelin maintenance. Thus, restoration of alkaline and low calcium conditions could be a key to improving catalase levels in support of restoring antioxidant balance.

5) Glucose levels are commonly elevated in cancer. Such a rise in glucose is a key enabler for cancer proliferation, as cancer commonly relies on glycolytic metabolism, in which many sugar molecules are split to meet energy demands. In the process of this glycolytic-centric metabolism, cancer produces a lactate “oxygen debt” along with acid, the so-called Warburg effect.

A rise in bloodstream acidification or lower pH is one signal that is recognized to promote insulin resistance and impair sugar uptake (Souto et al. Metab Syndr Relat Disord. 2011 August; 9(4): 247-253) Although cancer is known to manipulate the macro-environment towards acidosis, it utilizes adaptations in glucose transport to overcome the insulin resistance limitation. As an example means to encourage non-cancerous cells to use more glucose to reduce glucose levels available to cancer, metformin has been successfully applied in some patients (Zi et al., Oncol Lett. 2018 January; 15(1): 683-690.). As an alternative approach, it is additionally recognized that correcting extracellular acidification, such as through broad metabolic chain correction and acid-base rebalancing, could provide another means to enhance glucose utility in healthy cells to reduce the fraction available to cancer.

A rise in cortisol is a second signal that contributes to elevated glucose as the liver uses cortisol as a signal to manage blood sugar. Cortisol is additionally referred to in the context of stress response. This is even appropriate at the cellular level as cortisol is produced in a partnership between mitochondria and the endoplasmic reticulum according to their states of stress (Picard, et al. Frontiers in Neuroendocrinology. 49. 10.1016). In this way, the net stress in organelles provides feedback to determine cortisol levels and blood sugar levels. Because cancer promotes an acidic state to impair glucose utility in normal cells, the body responds by raising cortisol levels. Thus, actions that reduce mitochondrial stress and endoplasmic reticulum stress might be expected to reduce cortisol levels, with corresponding effects to reduce blood glucose.

6) Fatigue is a major symptom that can be present during cancer treatment and also affects patients post-treatment. Although fatigue typically improves in the year after treatment completion, a significant minority of patients continue to experience fatigue for months or years after successful treatment. These fatigue effects can be traced to various failings in energy metabolism and interactions between energy signaling and signaling from chronic inflammation, for instance, as measured by CRP (Bower, Nat Rev Clin Oncol. 2014 October; 11(10): 597-609.).

As a system of dependent signals, inflammation induces a metabolic switch from energy-efficient oxidative phosphorylation to fast-acting, but less efficient, glycolytic energy. In contrast to short-lived anerobic glycolysis, such as in exercise, the glycolytic metabolism becomes chronic in spite of aerobic resources being available. As such, so-called aerobic glycolysis leads to a reduced ATP yield, increases reactive oxygen species; increases lactate and acid production, and the corresponding acid shift in the bloodstream reduces insulin sensitivity. In addition to decreasing intracellular pH, increased metabolic acid production increases H⁺ flow from the cell, which drives ionic exchange at the cell membrane to concentrate calcium in the cytosol. Further, the inefficient glycolytic metabolism results in a deficit of ATP, which impairs the SERCA pump that normally relocates intracellular calcium to the endoplasmic reticulum. Thus, cytosolic H+ and Ca²⁺ rise to suppress fatty acid metabolism support in the peroxisome and mitochondria. Metabolic stress also provides feedback to the thyroid to reduce circulating T3/T4 levels, which are major regulators of ATP production in the mitochondria.

To address these dysfunctions and alleviate fatigue, one should look to correct the underpinning failures. For instance, an alkaline diet with nutritional support targeted to “complete” the metabolic chain may provide means to resolve intracellular aerobic glycolysis to restore an alkaline condition to the bloodstream (Schwalfenberg, J Environ Public Health. 2012; 2012: 727630). In a similar way, an exercise stimulus might first increase metabolic acid production and then, through action of renal and respiratory response, promote an alkaline after-effect in the bloodstream (Moriguchi T, et al. Tohoku J Exp Med. 2004; 202(3):203-211.). Such alkalizing factors could further enhance insulin pairing to restored desired glucose utility in normal cells. If corrected, one might anticipate for instance, that a lower insulin/glucose ratio could be observed, as less insulin would be required to promote glucose uptake.

As another consideration, effects like diet and exercise that could influence the metabolic chain to be more complete and also affect the intracellular electrolytes, like calcium, could also help with metabolic recovery. For instance, exercise is recognized to produce a post-exercise alkalization phase, an ionic exchange with the intracellular, and the resolution of lactate burden. Such factors could help relieve the acid status within cells to restore an alkaline environment and correct intracellular calcium status to restore health in energy managing organelles such as the endoplasmic reticulum, golgi, peroxisomes, and mitochondria. As such, improvements in both glucose and fatty acid metabolism might be expected.

In the event of disruption in cortisol, whether high or low, one must consider that both dysfunctions stem from mitochondrial stress and/or endoplasmic reticulum stress (Mol Endocrinol. Mar. 1, 2013; 27(3): 384-393.). Thus, correcting the sources of these stressors, including pH and calcium, may restore cortisol control to better manage healthy glucose levels.

In thyroid dysfunction, the root of failure can be further appreciated at the intracellular level as the organelles and systems that manage T3 and T4 production are impaired; the endoplasmic reticulum and golgi organelles come under stress due to the pH and calcium disturbances while the peroxidase system is downregulated to avoid compounding already excessive oxidative stress (Indian J Endocrinol Metab. 2016 September-October; 20(5): 674-678./). To address such dysfunction, one might target completing the metabolic chain to reduce acid production and oxidative stress, along with correction of the disruption in calcium status.

Another disruption driving fatigue in cancer patients is related to oxygen delivery, where oxygen delivery is impaired, and aerobic glycolysis becomes compounded by hypoxia. Such impairment in oxygen delivery can be the result chronic vasoconstriction (Can J Cardiol. 2016 July; 32(7): 852-862.), where disruptions in calcium signaling in the endothelium serving healthy cells cause eNOS to relocate from the plasma membrane to the Golgi, so as to prevent vasodilation. Vasoconstriction can also be fueled by ROS and elevated calcium, which causes endothelin to emerge from the Golgi to drive constriction at the plasma membrane (Front Pharmacol. 2016; 7: 438.). Another source of impairment stems from anemia, where red blood cell production is impaired by disruptions in the bone marrow niche such as hypoxia, elevated calcium, ROS, and reduced pH (Gilreath J A, et al. Am J Hematol. 2014; 89(2):203-212). At the same time, mitochondrial dysfunction can impair promoting of ferritin products towards hemoglobin to support RBC growth. Hemoglobin itself can be affected as lower pH can interfere with hemoglobin's affinity for oxygen; the so-called Bohr effect (Trends in Biochemical Sciences Volume 2, Issue 11, November 1977, Pages 247-249). Hypercoagulation is another factor that affects circulation of oxygen. Erythrocyte sedimentation rate (ESR) and blood pressure can serve as measures of this. As calcium levels rise in the blood and inflammatory markers are elevated, platelets can become more “sticky” to increase blood viscosity and restrict blood flow in the smallest vessels. Vascular plaque can also pose a challenge to circulation in cancer patients, as stress signaling expedites plaque growth and narrowing of vessels (Can J Cardiol. 2016 July; 32(7): 852-862.). Suppression of nitric oxide signaling, which promotes vasoconstriction, also limits angiogenesis in the endothelium serving healthy cells as NO is a key part of enabling the cascade from HIF to VEGF to signal where oxygen is insufficient, and new vessels are needed (Indian J Endocrinol Metab. 2012 November-December; 16(6): 918-930). Thus restoration of mitochondrial health, corrections in the metabolic chain, and corrections in acid, calcium, and redox status are key to promoting blood oxygen improvements, with an end goal of increasing metabolic ATP yield.

7) Another common dysfunction in cancer involves a disturbance in the bone marrow microenvironment or “niche” (Leukemia. 2008 May; 22(5): 941-950.). Simply put, the bone marrow niche is an incubator for many human cells. These can include red blood cells (RBCs), immune cells, and even platelets, which all originate in the bone marrow from the same progenitor cell, the hematopoietic stem cell (HSC) (Birbrair, Alexander; Frenette, Paul S. (2016-03-01). “Niche heterogeneity in the bone marrow.” Annals of the New York Academy of Sciences. 1370 (1): 82-96. ISSN 1749-6632. PMC 4938003. PMID 27015419). The bone marrow niche also supports Mesenchymal stem cells (MSCs), which transform to bone-forming osteoblasts, cartilage-forming chondrocytes, and fat-storing adipocytes. In addition, cross talk between MSC and HCS cells also promote specific cell type outcomes, such as osteoblast progenitors transforming to bone destroying osteoclasts when adipocytes and macrophages signal stress. For all of these cases, the evolution of each cell produced is driven by the signaling and chemical environment that “incubated” it, including the status of oxygen, calcium, ROS, glucose, and acid-base condition. Thus with such complex interdependency, many pathological states can arise from disturbances in this niche environment. Accordingly, disturbances to the niche environment can include reduced oxygen (hypoxia), excessive ROS, disturbed glucose, a more acidic acid-base status, and an elevated calcium status. While all of the variations are desired under special circumstances to provide the variety of cells needed to maintain the body, it can also be grossly disturbed to grow cell types and numbers that are not in the interest of the body, but which can be to cancers advantage. Thus, correcting niche environmental factors are recognized as a means to reduce inflammation, stop destructive WBC response, and improve cancer outcomes.

One stimulus that can address the distorted progeny from a distorted bone marrow niche is exercise. Here, an acidic pH shift in the bloodstream caused by working cells is followed by an alkaline rebound as the renal and respiratory processes ensue. Such an alkaline shift has been shown to cull excessive mature neutrophils and monocytes and promote a higher ratio of M2 anti-inflammatory forms. This effect can be appreciated by knowing how the metabolic requirements of innate immune cells differ. For instance, neutrophils are more dependent on glycolysis to provide rapid bursts of energy that are necessary for ROS production. Monocytes can rely on glycolysis during their M1 inflammatory phase but switch towards mitochondrial oxidative phosphorylation during the M2 anti-inflammatory resolution phase. Lymphocytes, such as T cells, are largely reliant on oxidative phosphorylation (Redox Biol. 2017 August; 12: 50-57.). Note that the glycolytic metabolisms of neutrophils and M1 monocytes align with the glycolytic metabolism of cancer cells. Furthermore, a climate supporting glycolytic metabolism or metabolic chain supported oxidative phosphorylation can bias the fates of these cells towards survival or death. Modulation of WBC species towards anti-inflammatory forms with fewer neutrophils and monocytes have been shown to improve long-term survival outcomes in cancer (Technol Cancer Res Treat. 2018; 17: 1533033818802813.).

To address the source of disturbed WBC response directly, one could endeavor to make the niche itself more alkaline with less hypoxia, lower calcium, lower ROS. Such signaling, which could be supported through dietary, exercise, or supplemental methods involving oral or infused methods, for instance, would promote a higher fraction of immune quiescing anti-inflammatory WBC response. A quiesced immune system with less auto-immune response may possibly better discriminate signals when needed to provide a selective WBC response, such an elevated eosinophil response as might be desired if a patient were to be infected with Lyme disease.

Measures that address a disturbed bone marrow niche are, of course, relevant to cancers involving the bone, such as multiple myeloma. In such cases, converting the bone marrow from a state of dysfunction towards one which was more alkaline, less hypoxic, lower in calcium, and more balanced in ROS would favor chondrocyte and osteoblast activity and reduce osteoclast and adipose activity. Such effects would be expected to be beneficial to addressing bone disorders in cancer, such as in multiple myeloma (Ann N Y Acad Sci. 2016 January; 1364(1): 32-51.)

8) Disturbances in poly ADP-Ribose polymerase (PARP) utility are another mechanism exploited by cancer. In ordinary function, PARP is used to repair ‘nicks’ in DNA so that cells can recover and replicate even if minimal errors in DNA handling occur. Otherwise, errors would stimulate a response to kill the affected cells. Such an adaptation is important for certain cells to proliferate. For instance, if PARP inhibitors are administered, myelo suppression, anemia, and neutropenia can result. This may not be surprising as such cells are formed in conditions of metabolic challenge that may involve more “mistakes.” Cancer upregulates PARP to its advantage by fostering acidic conditions with impaired nicotinamide servicing. In this way, cancer blocks natural channels of PARP inhibition so that the normal checks and balances are removed; to replicate and mutate unchecked (Mirza M R, et al. Ann Oncol. 2018; 29(6):1366-1376.). This feature makes some cancers especially hard to treat as the fast-evolving cells make it hard to design a treatment response. To restore natural PARP inhibition with a goal of reducing mutation rates in cancer, one could consider correcting metabolic and mitochondrial disfunction to restore alkaline conditions and seeking means to elevate nicotinamide levels.

9) Angiogenesis is another mechanism that cancer exploits to its benefit. Angiogenesis is the process of growing new blood vessels. In the functioning system, a hypoxic region of the bloodstream generates hypoxia-inducible factor (HIF). If left unchecked, HIF will progress to promote vascular endothelial growth factor (VEGF) to initiate budding in the wall of the vessel where a new vessel extension is desired. As this process requires feedback to ensure that the desire is valid, a second signal called the von Hippel-Lindau factor (pVHL) must be simultaneously “blocked,” else it will promote the ubiquitination of HIF and stop progression towards vessel growth. In ordinary function, pVHL is blocked by nitric oxide, which is released by endothelial NOS (eNOS) upon observation of a pH shift such as during exercise.

Cancer drives angiogenic dysfunction by promoting inducible nitric oxide synthase (iNOS) dysfunction to generate chronic NO presentation (Redox Biol. 2015 December; 6: 334-343.). Thus, cancer can chronically block pVHL in a local area to allow HIF to progress to VEGF and grow vessels unchecked (Song Z J, et al. World J Gastroenterol. 2002; 8(4):591-595.). As a note, a similar mechanism is present in the so-called “wet” form of macular degeneration involving angiogenesis (J Clin Invest. 2001 Mar. 15; 107(6): 717-725.).

To correct angiogenic dysfunction in cancer, one could first target iNOS dysfunction. As one possible mechanism to elevate iNOS, cancer can elevate calcium to deplete D vitamins, which lead to increases in IL-17 that are known to stimulate iNOS activity. As another mechanism, the preferred glycolytic metabolism of cancer can reduce pH while elevating calcium to reduce adiponectin levels and allow further stimulation of iNOS. Thus actions to restore calcium levels, agonize calcium signaling, and restore alkaline conditions could reduce iNOS activity. To promote this, magnesium could be administered as an agonist for calcium. Alternately, if aerobic metabolism could be encouraged by exercise, metabolic chain enhancements, and means to improve oxygen servicing to reduce metabolic acid, increase ATP yield, correct intracellular calcium, and restore fatty acid metabolism, reductions in iNOS activity might be expected as well.

A second means to target angiogenic dysfunction in cancer can be through targeting HIF directly (Hu Y, et al. J Cell Biochem. 2013; 114(3):498-50). Because HIF is generated in response to hypoxic events, it is possible to increase oxygen levels and reduce stimulation of HIF (Med Oncol. 2016; 33(9): 101). This could potentially be accomplished by using alkaline conditions to promote higher hemoglobin affinity to oxygen, alkaline bone marrow with more oxygen to resolve anemia, correction of eNOS function to support vasodilation in the balance of the vasculature, and other means.

10) To directly challenge cancer, one could promote acidic stress with a potential to realize apoptosis in cancer (Cell Stress Chaperones. 2015 May; 20(3): 431-440.). A therapeutic approach could be to deliver a temporarily elevated acidic pH in the bloodstream or interstitial space adjacent to a tumor to decrease its intracellular pH. Such a stimulus may result in acidic stress and apoptosis in cancer cells. A normal, healthy adult cell has an intracellular pH (pHi) of −7.2 and extracellular pH (pHe) around 7.4, while cancer cells have a pHi of >7.4 and a pHe of 6.7-7.1 (J Cell Mol Med. 2010 April; 14(4): 771-794.). This lower extracellular pH limits the buffering capacity of HCO3−. In order to maintain this alteration in pH, there is an upregulation in the activity and the expression of H+ ATPases, Na+/H+ exchangers, and H+ efflux co-transporters. This alteration of intracellular pH >7.2 allows for increased proliferation of cells. In addition, this alters the metabolism of the cells to a Warburg effect (or “aerobic” glycolysis), which breaks down glucose into lactic acid in order to produce ATP. It also allows evasion of apoptosis, which may be overcome by coerced intracellular acidification.

11) To additionally challenge cancer, one could also endeavor to restore p53 function. In normal cells, p53 is part of the stress feedback process that eliminates unhealthy mitochondria and can progress to the death of the cell itself (Giorgi C, et al. Proc Natl Acad Sci U S A. 2015; 112(6):1779-1784.). In cancer, this relationship is dysfunctional as mutations disrupt the conventional function of promoting cell death (Cancers (Basel). 2011 March; 3(1): 994-1013.). Correction of Ca+2-depleted ER stores, such as through correction of metabolic dysfunction, may be a potential way of restoring p53 (tumor suppressor gene). Further observations on p53 as related to cancer: There is a strong selection for p53 in most cancers. Calcium pumps are dysregulated during cancer altering signaling cascades. The depletion of storage in the ER allows for resistance to apoptosis through a downregulation in the signaling to p53. (Monteith, Gregory R., et al. Journal of Biological Chemistry, vol. 287, no. 38, 2012, pp. 31666-73,) When there is a release of calcium from the ER, raising cytosolic calcium levels, it activated p53 and then downstream apoptotic genes (Lowe, Julie M., et al. Cancer Research, vol. 74, no. 8, April 2014, pp. 2182-92).

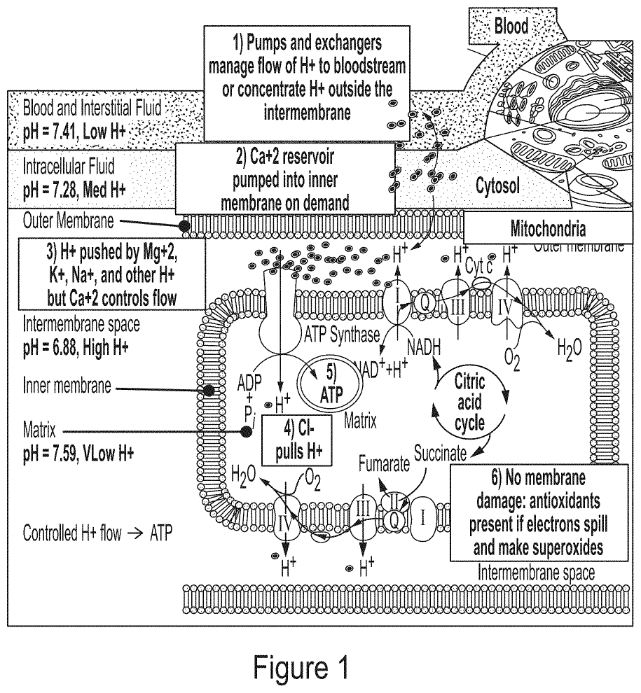

FIG. 1 depicts a diagram of the chemiosmotic gradient potential of hydrogen ions in a normally functioning mitochondria in a mammalian cell. As shown therein, blood and interstitial fluid typically has a pH of around 7.4, the intracellular fluid within a cell has a pH of around 7.28, and intermembrane space of mitochondria within the cell has a pH of around 6.88. Ionic pumps concentrate H⁺ ions in the intermembrane space of the mitochondria, resulting in a large H⁺ gradient between the intermembrane space and mitochondrial matrix across the inner membrane. The concentrations of other ionic species, such as Ca²⁺, Na⁺, K⁺, Mg²⁺, and Cl⁻ are also manipulated to create an electrochemical gradient across the various membranes, and intramitochondrial Ca²⁺, in particular, is important for managing the flow of H⁺ ions within the mitochondria. Hydrogen ions flow across the inner membrane into the mitochondrial matrix through ATP synthase, creating ATP from ADP. The electron transport chain is used to pump the H⁺ ions back across the inner membrane to maintain the proton gradient. A small percentage of electron transfer occurs directly to oxygen, leading to free-radical formation, which contributes to oxidative stress and may result in membrane damage if insufficient antioxidants are present.

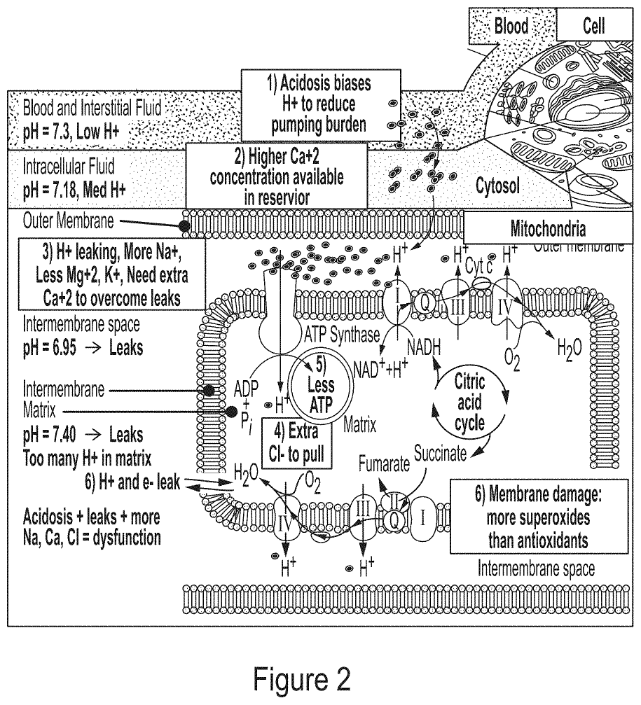

FIG. 2 depicts a diagram of the chemiosmotic gradient potential of hydrogen ions in mitochondria in a mammalian cell with a dysfunctional metabolism, as may occur after a prolonged exposure to a poor diet or lack of exercise. As shown in FIG. 2, the blood, interstitial space, and intracellular fluid have undergone acidotic shifts, i.e., increased the concentration of H⁺ ions and reduced the pH. At the same time, the pH in the mitochondrial matrix is increased from normal due to membrane leaks or reduced H+ ion pumping action from the electron chain transport. As a result, the net H⁺ electrochemical gradient available for the formation of ATP is reduced. Furthermore, the cell and mitochondria must increasingly rely on other ionic species to provide the necessary electrochemical gradient on demand, such as through higher than normal concentrations of Ca²⁺ within the intermembrane space “pushing” hydrogen ions across the inner membrane and a higher concentration of Cl⁻ within the mitochondrial matrix “pulling” the hydrogen ions. This dysfunctional ionic balance results in increased development of super oxidative species and increased membrane damage, and the metabolism of the cell slows down as a result. This reduces the amount of available ATP, causing a negatively reinforcing feedback loop that can lead to various adverse conditions and disorders.

A similar metabolic dysfunction occurs as a result of poor perfusion leading to a lactate burden, called metabolic acidosis in chronic state, which may be caused by, e.g., sepsis, multiple system atrophy (MSA), and ischemic conditions in peripheral limbs. For individuals incurring a chronic lactate burden, high blood levels of lactate steadily displace bicarbonate buffers to maintain acid-base homeostasis. A fraction of bicarbonate could then be removed by renal action to maintain homeostasis, and to reduce bloodstream bicarbonate levels. In addition, chronic disturbances in electrolytes can shift the setpoint for bicarbonate retention to additionally reduce stores. Such forces would, in turn, make less bicarbonate accessible for intracellular retention and intracellular buffering, ultimately reducing intracellular H⁺ stores. This reduction in H⁺ stores would require more Ca²⁺ to sustain a desired chemiosmotic gradient, leading to a dysfunctional ionic balance, as described above.

Stable therapeutic compositions of the present disclosure reduce the physiological bloodstream pH in a subject, and maintain that reduction in physiological bloodstream pH for a duration of time until renal and respiratory compensation processes negate the reduction, commonly followed by an alkaline “rebound.” The compositions of the present disclosure are formulated such that the formulated pH is below the physiologic norm (i.e., below 7.4). Bicarbonate concentration may, in some instances, be above physiologic norm (i.e., above 29 mM). The sudden influx of H⁺ ions, together with excess bicarbonate, and the manipulation of the electrochemical gradients allows for a return to normal mitochondrial metabolic processes, while other electrolytes, vitamin, and antioxidant support present in compositions of the present disclosure reduce the damage from oxidative stress. Other benefits of administration of compositions of the present disclosure include improvement of at least one of cardiovascular conditions, vasodilation, wound healing, vascular plaque, bicarbonate servicing, electrolyte economy, metabolic dysfunction, oxygen deficiency, Citric Acid Cycle, renal system operation, antioxidant dysfunction, angiogenesis, nitric oxide (NO) dysfunction, hormone function, and anemia.

In one embodiment of the invention, the compositions of the present invention are suitable for improvement of cardiovascular conditions by reducing or removing vascular plaques. Plaque forms in the arteries as a result of a number of factors, which are rooted in a wound-related signal dysfunction, including for example, lipid dysfunction, nitric oxide dysfunction and excessive ROS, which are caused, in part, by the presence of an acidic environment in the cells. For example, in an acidic environment, exogenous ROS levels become elevated. Smooth muscle contains several sources of ROS, which have been shown to function as important signaling molecules in the cardiovascular system. The elevated ROS signals to the smooth muscles to accrue in the arteries, as though recruited to fill wounds that do not actually exist. Additionally, in an acidic environment with ROS and an absence of nitric oxide, macrophages are signaled to respond to a non-existent threat, causing them to convert from the M1 to the M2 form, and begin sequestering lipids. The fat-laden lipids become accumulations of foam cells. Also, in an acidic environment, an endothelial nitric oxide synthase (eNOS) dysfunction occurs, causing an increased availability of arginase, which is necessary for the synthesis of collagen, and thus works with acid-pH stimulated action of fibroblasts to promote an accrual of collagen in the arteries. The elevations of retained intracellular Ca²⁺, and increases in unbound phosphate that occur from the metabolic dysfunction associated with an acidic environment (because less phosphate is complexed with ADP to form ATP), result in the promotion of calcific mineralized components of plaque. By restoring an alkaline environment in the cells, the compositions of the invention are able to reduce or reverse vascular plaque by correcting or improving at least one of, nitric oxide dysfunction (thereby restoring NO signaling), lipid dysfunction, eNOS dysfunction, reduction in smooth muscle recruiting, reduction of endogenous and exogenous reactive oxygen species (ROS), elevated Ca²⁺, or restoration of fatty acid metabolism. For example, upon the introduction of an alkaline environment, the smooth muscles, in the absence of the ROS signal, recognize the absence of a wound, and consequently, they down-regulate and begin to directionally orient towards their vasodilation and vasoconstriction tasks. Also, for example, in an alkaline, low ROS environment in the presence of eNOS nitric oxide signaling, foam cells are signaled to release their lipids. Along with the calcific plaque reversal or reduction, the suppleness of the vascular vessel returns. In addition, the acid-shifting action of the drug liberates atomic components of the mineral deposits, while magnesium in the composition of the invention aids in the prevention of plaque re-deposition, to reduce the hardening of the arteries from the mineral deposit components.

In one embodiment of the invention, the compositions of the present invention are suitable for preventing or minimizing hypoxia in a subject. The lack of sufficient oxygen reaching cells or tissues in a subject can occur even when blood flow is normal. This can cause many serious, sometimes life-threatening complications. Use of the compositions of the invention enables the resolution or improvement of conditions commonly associated with hypoxia, such as, for example, heart attack, cardiovascular problems, lung conditions, concussive cascade, reperfusion injury, myocardial infarction, hypoxia associated with diabetes, tissue trauma, and the like. Many of these conditions are associated with vasoconstriction. The composition can counteract such vasoconstriction by promoting vasodilation via at least one of three pathways, namely endothelin, prostacyclin, or NO-soluble guanylyl cyclase (NO-sGC). For the endothelin pathway, the compositions elevate Mg²⁺ in the bloodstream to antagonize Ca²⁺. This blocks Ca²⁺ from potentiating vasoconstriction, allowing the arteries to relax and dilate. Meanwhile, the compositions also provide metabolic corrections to reduce metabolic sources of ROS and reduce the presentation of endothelin stimulants at the cell surface, thereby reversing Ca²⁺ overstimulation. For the pro stacyclin pathway, niacinamide in the composition elevates adenosine 3′,5′-cyclic monophosphate (cAMP) activity, which completes prostacyclin potentiation towards vasodilation. For the NO-sGC pathway, as noted above, the compositions of the invention provide a gradient of H⁺ flowing into the cells to promote Ca²⁺ efflux, which corrects elevated Ca²⁺ presentation. One effect of high levels of Ca²⁺ is the elevation of caveolin. As the caveolin elevate, they take residence in the caveolae on the cell surface, causing the displacement of eNOS, which migrates to the Golgi system. The combination of low ROS and low intracellular Ca²⁺ achievable using the composition of the invention allows eNOS, to return from the Golgi to the cell membrane, thereby restoring eNOS's ability to promote vasodilation. As the eNOS returns to the membrane, the bloodstream pH shifts, promoting NO release via the NO-sGC pathway, and promoting vasodilation. In addition, renal responses to rebalance pH produce a second “pH shift” towards alkaline, once again stimulating NO/NO-sGC vasodilation to extend the duration of the effect.

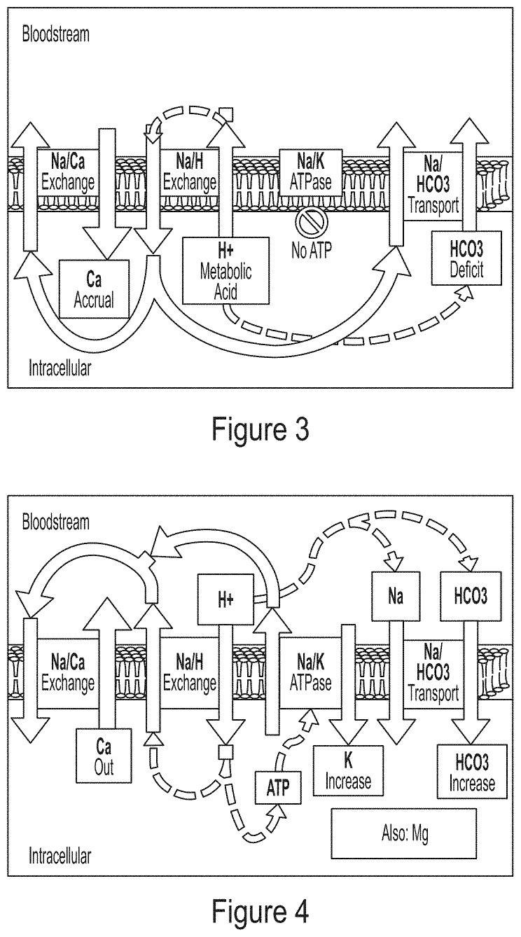

As shown in FIG. 3, when a subject's body is under a state of metabolic crisis, such as a hypoxic crisis, intracellular acidification drives the intracellular accrual of Ca²⁺. This occurs because adenosine triphosphate (ATP) is required to resolve the sodium burden created as H⁺ leaves the cell. However, in the hypoxic state, ATP becomes impaired, and as a consequence, the Na⁺/K⁺ ATPase pump becomes inactive. The Ca²⁺/Na⁺ exchange must resolve the Na⁺ burden by accumulating Ca²⁺ in the cell. To reverse this process, the hypoxic state must be resolved to restore ATP production (and Na⁺/K⁺ ATPase), or extracellular H⁺ must be presented. As shown in FIG. 4, the compositions of the invention achieve both of these things, enabling the rapid resolution of the Ca²⁺ overburden and the corresponding metabolic crisis. The composition adjusts the pH of the bloodstream, acidifying it, and in doing so, causes H⁺ to enter through the Na⁺/H⁺ exchange route. As the H⁺ enters, it pushes Na⁺ out. As noted above, the composition of the invention promotes vessel vasodilation to improve blood flow. With this increased blood flow comes increased oxygen, entering, which enables the creation of ATP through aerobic metabolism. The composition also elevates Mg²⁺ in the bloodstream. The increased Mg²⁺ facilitates the transport of the ATP, as Mg-ATP, to the Na⁺/K⁺ ATPase, providing the stimulus to push Na⁺ out. Some of the increased Na⁺ in the bloodstream reenters through the Ca²⁺/Na⁺ exchange. Additionally, the bloodstream presentation of H⁺, in concert with elevated bloodstream bicarbonate, promotes bicarbonate entry into the cell. This process provides an antidote to reverse calcium accrual in the cell, improving the cells' capacity to restore a chemiosmotic gradient with less reliance on Ca²⁺ and more utility of HCO₃⁻ buffered H⁺ to ultimately reduce metabolic acid burden and metabolic ROS, to promote restoration of the intracellular towards alkaline, with improved redox status. The steady biasing towards alkaline and low ROS promotes positive rebalancing of electrolytes and pH in the cytosol, organelles, lysosomes, peroxisomes, calcium status, magnesium status and ROS status within the cell. Additionally, it changes the cellular economy to restore potassium and bicarbonate, while at the same time reducing intracellular calcium.

The vasodilation that can be achieved by use of the composition of the invention makes the composition useful for wound care. It was unexpectedly discovered that use of the compositions of the invention may provide wound recovery even in subjects who have exhausted conventional treatment methods, including those with gangrenous presentation, or chronic, diabetic or traumatic wounds. Metabolic changes are among the effects observed following traumatic injury and surgical trauma. These include inflammatory responses, which trigger constriction of blood flow to the affected regions. While this advantageously minimizes blood loss at the site of an open wound or internal bleed, it may impair healing by promoting a hypoxic intracellular environment. In trauma situations where bleeding risk is absent or reduced (for example, by compression), it may be desired to suppress the inflammatory response, to avoid secondary injuries from hypoxia. In cases of chronic inflammation, such as with chronic critical limb ischemia (CLI), the suppression of inflammation can expedite healing. The vasodilation promotion and improved perfusion caused by the composition of the invention contribute towards breaking the cycle of inflammation. In addition to promoting vasodilation in order to increase oxygen servicing, the compositions of the invention are also capable of correcting key metabolic aberrancies that are present in wounds. The compositions may, for example, improve at least one of restoring acid-buffer status and correction of elevated Ca²⁺; reducing metabolic sourced ROS; correcting acidosis; correcting over-active iNOS and restoration of eNOS and nNOS function; promotion of beneficial angiogenesis after eNOS is corrected; and suppression of iNOS promoted aberrant angiogenesis, all of which are important for wound care.

Because H⁺ also administrates acetylcholine uptake, which is part of muscle support, and is a part of the cerebellum control process, and ATP is relevant for all of these systems, disorders of the central nervous system are another treatment target. Additionally, action to resolve intracellular acid, calcium accrual, reduced ROS, and increased Mg, are factors that can enhance function in the peroxisome, to better maintain catalase antioxidant supply, and additionally support the lipid modeling required for myelin maintenance of nerve sheaths.

In some instances, the reduction in physiologic bloodstream pH caused by the composition of the invention may be minimal, or not observed, due to the particular formulation of an administered composition, the rate at which a composition is administered, or both. However, the therapeutic benefits described herein may still be achieved due to the net elevation of bicarbonate concentration that occurs. Due to an excess of H⁺ upon administration, the body prioritizes retention of, and augmentation of, the buffer components (e.g., bicarbonate), as acid balancing processes proceed. Thus, a greater fraction of the buffering agent is retained within the cells and bloodstream as the system alkalinizes and returns the physiological pH towards baseline. Such an “alkaline rebound” may result in bloodstream pH overshooting slightly for a net alkaline stabilization relative to the starting pH. The “alkaline rebound” achieves a higher residual concentration of intercellular and bloodstream buffer components, including bicarbonate. Alternatively, the system may regulate to a final pH equivalent to that present prior to treatment, but with bloodstream buffering, with regard to acidic species, being increased. Alternately, the bloodstream pH may settle to be more acidic than prior to the treatment, yet while a variety of aforementioned exchange phenomena are promoted. In contrast to infusion of a simple buffer, such as bicarbonate, in the absence of acidic components, co-administration of acid and buffer is key to limiting the H⁺ efflux rate, while the intracellular calcium correction is achieved.

In one embodiment of the invention, the compositions of the present invention are suitable for increasing nitric oxide synthase (NOS) in a subject. The pH biasing and increase in bicarbonate concentration as provided by compositions of the present disclosure (including decreases in pH upon administration and “alkaline rebounds” as homeostasis is restored) may also restore endothelial and neuronal NOS, leading to a selective increase in nitric oxide production. Nitric oxide is a gaseous signaling molecule with a role in, e.g., hemostasis, smooth muscle (particularly surrounding vasculature), neuronal signaling, and in the gastrointestinal tract. NO has been implicated in a variety of physiological systems, and the increased levels resulting from administration of the compositions described herein may serve a role in providing the therapeutic benefits described herein. For example, in glaucoma, NO may play a role in regulating intraocular pressure via the trabecular meshwork. In atherosclerotic plaques, NO stops the aberrant perpetuation of smooth muscle recruitment, foam cell accrual and lipid storage, and collagen deposition, and it may ultimately lead to reversal of plaque damage and a return of the vascular section to physiological norms.

In one embodiment of the invention, the compositions of the present invention are suitable for reducing lactate burden in a subject in need thereof. As used herein, the term “lactate burden” means any physiological condition characterized by elevated lactate levels. This may include, for example, and without limitation, chronic lactate burdens such as acidosis, sepsis, and MSA, or acute lactate burdens such as may occur during and after physical exertion such as exercise. Lactate circulating oxygen debt burden that is retained in muscles can be stimulated to be released by bicarbonate, and subsequently metabolized, thus lowering the subject's lactate burden. The ability to eliminate lactate burden is important for a subject who has had, for example, an organ transplant. Where the transplant procedure involves the use of citrate anticoagulant, the citrate must be metabolized. This metabolization can induce a lactate burden in those individuals. Additionally, lactate burden is a component of sepsis and a chronic burden in diabetics. In the above instances, as well as in others involving a lactate burden, the use of the compositions of the invention may reduce that burden.

In one embodiment of the invention, the compositions of the present invention are suitable for reducing acidosis in a subject in need thereof, by administering to the subject the composition of the invention. One of the metabolic effects of trauma is the suppression of insulin, resulting in a reduction of the normal anabolic effect of insulin towards an increase in catabolic effects. This leads to a shift towards free fatty acids as the primary source of energy, with triglycerides providing 50 to 80% of the energetic need. Reducing the catabolic response encourages faster healing after surgery. These same mechanisms are in play in the diabetic patient and become a larger challenge as subjects progress in their metabolic dysfunction. Underlying this catabolic process are aberrations in the metabolic chain that tend towards incomplete oxidation, leading to an increase in acidic products and an elevation of ROS from metabolic sources. As noted hereinabove, in trauma, this catabolic shift is driven by the hypoxic state, as inflammation and the vasoconstrictive response impair circulation. In diabetes, the shift is marked by glucose intolerance, and compounded by plaque-induced circulatory impairments and a sedentary lifestyle. In both cases, incomplete oxidation results in acidification in the cell and the promotion of transport biases which cause Ca²⁺ to concentrate in the cytosol. This concentration of Ca²⁺ cascades to the mitochondrial inner-membrane so that Ca²⁺ takes on a larger role in the chemiosmotic gradient, reducing the role of H⁺ itself. Such a shift in Ca²⁺ and H⁺ initiates a progressive shutdown in the electron chain transport (ECT), so that Ca²⁺ takes on a greater role in controlling the chemiosmotic potential. This also leads to an increase in metabolic ROS from ECT stages. Over time, impaired circulation reduces B-vitamin servicing, which impairs both the Krebs cycle and ECT, further increasing metabolic ROS. At the same time, impaired circulatory servicing reduces antioxidant maintenance to leave the elevation in ROS unchecked. While such aberrations have beneficial qualities, such as promoting the creation of NAPDH oxidases for bactericidal function during infection, they also present impairment to the healing process, as they promote catabolism. Furthermore, a balance of signals including acidosis, hypoxia, Ca²⁺, ROS, and iNOS/NO, collectively suppress emergence of M2 macrophages, as desired, to promote healing. To address these aberrancies, the composition of the invention facilitates Ca²⁺ correction and enhances B-vitamin servicing and ascorbic acid anti-oxidant servicing via elevated presentation. Additionally, acid burden is reduced, promoting an alkaline bias. Elevated HCO₃⁻ buffer levels also serve to preserve this alkaline bias.

The elements of metabolism referenced above also affect insulin management. For example, insulin release is stimulated from the pancreas when a signal of elevated Ca²⁺ is released to the bloodstream. For Ca²⁺ to be released to the pancreas, hydrogens must be created, through incomplete metabolism, to displace Ca²⁺ from the cytosol to the bloodstream. As noted herein-above, the Na⁺/K⁺ ATPase must be served with Mg²⁺ and ATP to facilitate the flooding Na⁺ to the bloodstream to ultimately stimulate the Na⁺/Ca²⁺ exchanger to release Ca²⁺ to the bloodstream. Additionally, for sensing of elevation to occur, the background level of Ca²⁺ in the bloodstream needs to be low enough for the pancreas to observe the change. In acidosis, this would be impaired as Ca²⁺ solubility is elevated in the blood and in the cytosol. As a further example, ROS, such as peroxide, can promote insulin function, when presented at low levels, and prevent presentation and action of insulin when presented at high levels. Thus, correction of acidosis and enhancement of Mg²⁺ is key to restore insulin management. So too are suppression of ROS (e.g., H₂O₂) through antioxidant support and facilitation of TCA and ECT function to achieve near-complete oxidation of Acetyl-CoA to CO₂ and H₂O.

### II. Compositions

In some embodiments, the composition, as described herein, is a stable therapeutic composition that has been formulated to make it suitable for intravenous administration to a subject. The composition includes an intravenous buffer solution, containing at least one pharmaceutical grade acid, and at least one pharmaceutical grade pH buffering agent. To ensure their suitability for pharmaceutical use, the acid solution and the buffer solution are present in a sterile aqueous solution. The concentration of the pharmaceutical grade acid and the pharmaceutical grade pH buffering agent in the buffer solution is sufficient to provide a total titratable acid content between 60 mmol/L and 3,000 mmol/L when administered to a subject. The acid and base are selected so that they together are able to provide a buffer solution having a pH of between 4.0 and 7.7.

In some embodiments, the concentration of the pharmaceutical grade acid and the pharmaceutical grade pH buffering agent in the buffer solution is sufficient to provide a total titratable acid content between about 40 mmol/L and about 3,000 mmol/L (e.g., between about 80 mmol/L and about 2,500 mmol/L, between about 100 mmol/L and about 2,000 mmol/L, between about 150 mmol/L and about 1,500 mmol/L), when administered to a subject, where the buffer solution is effective to provide a buffer solution pH of less than 5.5. In one example, the concentration of the pharmaceutical grade acid and the pharmaceutical grade pH buffering agent in the buffer solution is sufficient to provide a total titratable acid content of from about 100 mmol/L to about 2,000 mmol/L when administered to a subject, where the buffer solution is effective to provide a buffer solution pH of less than 5.5. In another example, the concentration of the pharmaceutical grade acid and the pharmaceutical grade pH buffering agent in the buffer solution is sufficient to provide a total titratable acid content of from about 200 mmol/L to about 1,000 mmol/L when administered to a subject, where the buffer solution is effective to provide a buffer solution pH of less than 5.5.

In one embodiment of the invention, the concentration of the pharmaceutical grade acid and the pharmaceutical grade pH buffering agent in the buffer solution is sufficient to provide a total titratable acid content between about 40 mmol/L and about 3,000 mmol/L when administered to a subject, where the buffer solution is effective to provide a buffer solution pH of less than greater than or equal to 5.5. In another embodiment of the invention, the concentration of the pharmaceutical grade acid and the pharmaceutical grade pH buffering agent in the buffer solution is sufficient to provide a total titratable acid content between about 60 mmol/L and about 2,000 mmol/L when administered to a subject, where the buffer solution is effective to provide a buffer solution pH of less than greater than or equal to 5.5.

In another embodiment of the invention, the concentration of the pharmaceutical grade acid and the pharmaceutical grade pH buffering agent in the buffer solution is sufficient to provide a total titratable acid content between about 80 mmol/L and about 3,000 mmol/L when administered to a subject, where the buffer solution is effective to provide a buffer solution pH of less than greater than or equal to 5.5.

An acid is a molecule or ion that is capable of donating a hydrogen ion H⁺. The amount of H⁺ ions in a solution is measured by its pH, where a pH of less than 7 constitutes an acidic pH. Humans typically have a bloodstream pH of 7.4. Compositions of the present disclosure comprise an acid that provides an amount of H⁺ ions to decrease the physiological bloodstream pH in a subject. Without being bound to any theory, it is believed compositions of the present disclosure increase the H⁺ gradient in various cellular environments, including, e.g., mitochondria. This increased mitochondrial H⁺ gradient drives higher production of ATP and, through other physiological homeostatic systems, causes changes in concentration gradients of the cellular membranes, which in turn rebalances physiological ions such as sodium, magnesium, potassium, and calcium. For example, an increased H⁺ gradient in the bloodstream may stimulate calcium pumps in cellular membranes, thereby increasing intracellular H⁺ and reducing intracellular Ca²⁺. The concentration gradients of sodium, magnesium, and potassium are also affected. By manipulating ionic gradients using compositions of the present disclosure, many conditions and diseases and symptoms thereof may be treated, ameliorated, or prevented.

In some embodiments, compositions of the present disclosure are sufficient to reduce the bloodstream pH of a subject by a small, moderate, or large amount. The representative formulations and dosage of the composition are described in Table 1. In some embodiments, the amount of acid in a composition of the present disclosure is sufficient to reduce the bloodstream pH of a subject by about 0.01, 0.02, 0.03, 0.04, 0.05, 0.06, 0.07, 0.08, 0.09, 0.1, 0.15, 0.2, 0.3, 0.4, 0.5, 0.6, 0.7, 0.8, 0.9, 1.0, or 1.1, or more. The reduction in pH may also be expressed by the desired pH level of the bloodstream after administration of a composition of the present disclosure, e.g., 7.2. In some embodiments, a composition of the present disclosure comprises sufficient acid to reduce the bloodstream pH of a subject to about 7.3, 7.2, 7.1, 7.0, 6.9, 6.8, 6.7, 6.6, 6.5, 6.4, or 6.3. A reduction of bloodstream pH to below 6.3 is not typically advised, as it may pose a cell health risk and threaten the integrity of cellular phospholipid bilayers. In cases of alkalosis where nominal pH may exceed 7.4, a “reduction” in pH provided by administration may still result in a bloodstream pH exceeding 7.4. For example, administration of a composition of the present disclosure may shift the physiological pH from about 7.7 to about 7.5.

Compositions of the present disclosure may contain one or more pharmaceutical grade acids. In some embodiments, compositions of the present disclosure comprise a mixture of one or more pharmaceutical grade acids. Acids may include any physiologically acceptable acid, including, without limitation, hydrochloric acid, ascorbic acid, citric acid, lactic acid, phosphoric acid, or combinations thereof. The pH of a composition of the present disclosure may be between about 4 and about 7.7. In some embodiments, the pH of a composition of the present disclosure is between about 6.1. In embodiments where the pH of the composition is very low, the rate of administration may have to be managed to avoid tissue damage adjacent to the injection site as dilution is effected in the bloodstream.

In another aspect, compositions of the present disclosure comprise a pH buffering agent. A pH buffering agent is a weak acid or base that is used to maintain the pH of a solution near a desired value. Compositions of the present disclosure comprise a pH buffering agent such that the reduction in bloodstream pH may be sustained for a desired duration. In some embodiments, the pH buffering agent may comprise a conjugate acid or a conjugate base. In some embodiments, the pH buffering agent may comprise any physiological acceptable buffering agent, including, without limitation, sodium bicarbonate, a phosphate buffer, citrate buffer, or a synthetic buffer creating specific alkaline conditions (e.g., tris-hydroxymethyl aminomethane), or combinations thereof.

The buffer capacity of a solution is a measure of the solution's ability to resist pH change, i.e., to maintain a specific pH level. As discussed above, acid-base homeostasis relates to the proper balance of acids and bases in extracellular fluids, i.e., the pH of the extracellular fluid. In humans, the pH of plasma is approximately 7.4 and is tightly maintained around that value by three interconnected systems: 1) buffering agents, including bicarbonate, phosphate, and proteins), 2) the respiratory system, which impacts the partial pressure of carbon dioxide in blood plasma, and 3) the renal system, which excretes waste acids and bases. Accordingly, in some embodiments, compositions of the present disclosure comprise a pH buffering agent in order to maintain the desired bloodstream pH level below the typical pH value of about 7.4 in the face of pressures exerted by the physiological systems that regulate acid-base homeostasis.

In some embodiments, compositions of the present disclosure comprise a pH buffering agent in an amount sufficient to maintain the reduction in bloodstream pH or to maintain the desired pH level, for a duration of 1 minute to 1 week. The desired duration of the reduced bloodstream pH level will depend on the particular indication being treated as well as the individual being treated. In some embodiments, a small, moderate, or large buffer capacity may be desired. In one means of administration, a small quantity of drug and/or a slow administration of a drug product could stimulate compensatory processes that can be respiratory or renal, so as to mitigate observable acid shifting potential, but having stimulated respiratory and renal activity. In such cases, a bloodstream response may be neutral or may tend toward alkaline. Alternatively, administration of a high dose, and/or a dose with a fast administration rate, such as a bolus or fast IV drip could introduce the acid and overwhelm the compensatory processes to yield an observable downstream pH toward acidic. Such a stimulus would commonly be expected to be followed by a rebound of bloodstream pH towards alkaline throughout the treatment or post-treatment. The outcome resulting from a given dose level and/or administration rate may be different from patient to patient and from administration to administration as the patient's health, electrolytic status, pH status, and compensatory process status evolve. Different buffer capacities may be sufficient to maintain the reduction in bloodstream pH for a duration of 1 minute to 1 week.

In other embodiments, the buffer capacity may also be expressed in molar equivalent of common buffers, such as bicarbonate. In some embodiments, the composition has a buffer capacity between 0.1 mM HCO₃⁻ equivalent and 1,200 mM HCO₃⁻ equivalent. In other embodiments, the buffer capacity is between 0.1 mM HCO₃⁻ equivalent and 10 mM HCO₃⁻ equivalent. In some embodiments, the buffer capacity is between 10 mM HCO₃⁻ equivalent and 50 mM HCO₃⁻ equivalent.

In some embodiments, the buffer capacity is between 10 mM HCO₃⁻ equivalent and 1,000 mM HCO₃⁻ equivalent. In some embodiments, the buffer capacity is between 50 mM HCO₃⁻ equivalent and 800 mM HCO₃⁻ equivalent. In some embodiments, the buffer capacity is between 100 mM HCO₃⁻ equivalent and 600 mM HCO₃⁻ equivalent. In some embodiments, the buffer capacity is between 200 mM HCO₃⁻ equivalent and 550 mM HCO₃⁻ equivalent. In some embodiments, the buffer capacity is between 20 mM HCO₃⁻ equivalent and 100 mM HCO₃⁻ equivalent. In other embodiments, buffer capacity may be expressed by the molar concentration of HCO₃⁻, or other common buffers. For example, in some embodiments, the molar concentration of HCO₃⁻ may be between 0.01 molar and 10 M. In other embodiments, the molar concentration of HCO₃⁻ may be between 0.5 and 2 M.

In another embodiment, the present disclosure provides a composition having a pH below physiological pH (i.e., below 7.4) and an HCO₃⁻ concentration above physiological levels (i.e., above 29 mM). In some embodiments, the pH of the composition may be between 4 and 7.7, and the HCO₃⁻ concentration may be between 30 mM and 2 M). In other embodiments, the pH of the composition may be between 5.5 and 7.4. In further embodiments, the pH of the composition may be around 6.

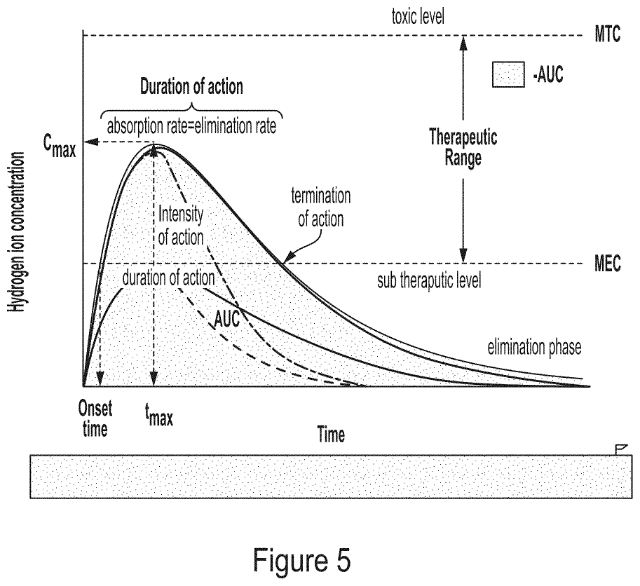

FIG. 5 shows a diagram of the amplitude and duration of an acid state shift caused by different formulations of compositions of the present disclosure. The black lines, both solid and dotted, depict a large acid shift, i.e., a composition with a high concentration of H⁺ ions. However, the buffering capacity of the composition depicted by the dotted black line is smaller than that of the solid line, such that the acid shift is maintained for a shorter duration. The gray lines, both solid and dotted, depict a smaller acid shift, i.e., a composition with a lower concentration of H⁺ ions. Again, the buffer capacity between these compositions varies such that the acid shift caused by the composition depicted by the dotted gray line is maintained for a shorter duration. Compositions of the present disclosure may be designed along these two spectrums, amplitude of shift and duration of shift, according to desired therapeutic properties and administration schedules.

In another embodiment, the present disclosure provides a stable therapeutic composition comprising a buffer solution comprising a pharmaceutical grade base and at least one pharmaceutical grade conjugate acid, wherein the buffer solution is sufficient to raise the physiological bloodstream pH of a subject by 0.1 to 1.1, and wherein the buffer solution has a buffer capacity sufficient to sustain the elevation of the physiological bloodstream pH. In some embodiments, the buffer capacity may be sustained for a period of time, for example, 1 minute or 1 week. The compositions may further comprise vitamins, salts, acids, amino acids or salts thereof, and stabilized oxidative species.

In another aspect, compositions of the present disclosure may further comprise salts to provide sources of physiological relevant ionic species, such as Na⁺, K⁺, Mg²⁺, Cl⁻, PO₄³⁻, or Ca²⁺. These may include, without limitation, sodium chloride, disodium phosphate, potassium chloride, monopotassium phosphate, magnesium chloride, and calcium chloride. The compositions may further comprise other trace elements and their salts, including, but not limited to, selenium, copper, chromium, iodine, fluoride, zinc, manganese, molybdenum, and iron.

Sodium ions are required in relatively large concentrations for normal physiological functioning. It is the major cation of the extracellular fluid. It plays an important role in many physiological processes, including the regulation of blood volume, blood pressure, osmotic equilibrium, and pH, as well as the generation of nerve impulses.

Potassium ions are the major cation of intracellular fluid, and, with the sodium ions of the extracellular fluid, is a primary generator of the electrical potential across cellular membranes. Accordingly, it plays a significant role in normal functioning and is critical in such body functions as neurotransmission, muscle contraction, and heart function.

Calcium ions are likewise important to many physiological processes. In particular, Ca²⁺ ions are one of the most widespread second messengers used in signal transduction. In endothelial cells, Ca²⁺ ions may regulate several signaling pathways, which cause smooth muscles surrounding blood vessels to relax. Dysfunction within Ca²⁺-activated pathways can lead to an increase in tone caused by unregulated smooth muscle contraction. This type of dysfunction can be seen in cardiovascular diseases, hypertension, and diabetes.

Magnesium ions are required in relatively large concentrations in normal metabolism. It is recognized that deficiency of magnesium is rare unless it is accompanied by severe losses in other electrolytes such as in vomiting and diarrhea. It is, however, frequently recognized as deficient in the modern diet with symptoms such as muscle tremors and weakness. This mineral is important in many enzymatic reactions and will stabilize excitable membranes. Administered intravenously, magnesium may produce an anesthetic action, and this is indirect evidence of its action on the vascular wall endothelial component to stabilize and normalize the surface of the vascular wall.

In some embodiments, a composition of the present disclosure comprises Na⁺ at a concentration between 0.1 mM and 1 M. In other embodiments, a composition of the present disclosure comprises K⁺ at a concentration between 0.0 mM and 1 M. In some embodiments, a composition of the present disclosure comprises Mg²⁺ at a concentration between 0.1 mM and 1 M. In other embodiments, a composition of the present disclosure comprises Ca²⁺ at a concentration between 0.1 mM and 1 M.

As described above, the interplay between the various ionic species is disrupted in various physiological conditions, and compositions of the present disclosure may include these species to aid in the restoration of normal physiological conditions and concentrations. For example, high intracellular Ca²⁺ may be restored to a lower level as offset by Mg²⁺, K⁺, and H⁺, which may lead to NOS presentation in the cytosol and restoration of NO levels.

As stated above, the compositions described herein may include vitamins and vitamers, which is a substance(s) that has vitamin-like activity. Vitamins selected from the group consisting of the water-soluble and lipid-soluble group, and a combination of two or more thereof may also be added to the pharmaceutical composition. Preferably, the pharmaceutical composition includes ascorbic acid. Ascorbic acid is included as a strong antioxidant component and to maintain the structural integrity of connective tissue, including epithelial basement membranes and to promote wound healing. It may also play a distinct role as an agent with strong anti-inflammatory actions. The oxidized form of the vitamin, dehydroascorbic acid, has been shown to transfer intracellularly where some of it is reduced within the cell via action of glutathione. Deficiencies of the B group of vitamins, as well as A and E, are also protected by ascorbic acid and corresponding interactions of dehydroascorbic acid and glutathione. In some embodiments, a composition of the present disclosure comprises dehydroascorbic acid, an oxidized form of ascorbic acid that is actively imported into the endoplasmic reticulum of cells via glucose transporters. Presentation of dehydroascorbic acid can also stimulate production of glutathione in the liver, which facilitates recycling of dehydroascorbic acid into ascorbic acid. Thus, dehydroascorbic acid indirectly enhances intracellular antioxidant resources. Dehydroascorbic acid may be present via direct inclusion of pharmaceutical grade dehydroascorbic acid, or by conversion of ascorbic acid via contact with a reactive oxygen species such as HOCl, H₂O₂, or OCl.

The B Group of Vitamins has been shown to be important in human food intake and plays an important role acting as co-enzymes in cellular metabolism and energy production. The entire B group of vitamins may be included in the formulation to address any deficiencies in the patient population to be treated.

The B group vitamins are found to occur naturally together in foods and are generally included comprehensively for this reason. The B group includes: 1) Thiamine (B1), which plays an important role in energy production within the cell, specifically as co-enzyme in metabolism of carbohydrates. At least 24 enzymes are known to use thiamine as a co-enzyme; 2) Riboflavin (B2) in the form of flavin mononucleotide and flavin adenine dinucleotide are part of all dehydrogenase enzymes. Deficiency of this vitamin causes inflammation of the mouth, tongue, dermatitis, defective vision, and blood dyscrasias; 3) Niacinamide (B3) is included as part of the B group of vitamins as deficiency syndromes in clinical pellagra are well known clinical manifestations of deficiencies. The deficiency states of this vitamin are associated with intestinal diseases and alcohol misuse. It also occurs in diabetes mellitus and carcinoid syndrome. The active forms of this vitamin include the nicotinamide dinucleotides NAD and NADP, which are the co-enzymes and co-substrates for numerous dehydrogenases responsible for oxidation-reduction systems within the human cell, which are indispensable for energy production. The formation of nicotinic acid from the administered nicotinamide in the formulation produces nicotinic acid possessing additional actions not shared by nicotinamide, such as inhibition of cholesterol synthesis; 4) Calcium D-Pantothenate (B5), pantothenic acid forms a major part of the molecule of co-enzyme A, which is important in the energy-producing metabolic cycles in the mitochondria of all cells. The effect of this vitamin on various disease syndromes has been recognized. Such as its use in neurotoxicity produced by streptomycin and its use in diabetic neuropathy, skin diseases, and adynamic ileus; and 5) Pyridoxine (B6) is widely utilized as a co-enzyme in over 40 types of enzymatic reactions. The B Group of vitamins may also aid in providing an increase of antioxidants and stimulated glutathione to reduce reactive oxygen species, which ultimately aids in NO expression.

The most important of these are the transamination reactions and the influence of pyridoxine on tryptophan metabolism. Kynureminase, which is an enzyme used to identify pyridoxine deficiencies, loses its activity when pyridoxine is not present and may result in secondary nicotinic acid deficiency as a result of lack of the kynureninase conversion of nicotinic acid from tryptophan.

Cyanocobalamin (B12) is used because of the frequent reports of mal-absorption of cyanocobalamin, caused by poor dietary habits, senescence, and certain drugs (metformin) used as a hypoglycemic agent in diabetes mellitus. This vitamin is essential for normal erythropoiesis to occur, and recent findings have also implicated this vitamin with improvement of neuronal transmission in motor neuron disease. (Rosenfeld, Jeffrey, and Ellis, Amy, 2008, Nutrition and Dietary Supplements in Motor Neuron Disease, Phys Med Rehabil Clin N Am., 19(3):573-589).

Vitamin K is a fat-soluble vitamin. There are two naturally occurring forms of the vitamin. Vitamin K1 is the dietary Vitamin K and is abundant in green leafy vegetables, whereas vitamin K2 is present in tissues. Vitamin K2 is synthesized by bacteria. It is found mainly in fermented products like fermented soybeans, cheese, curds and to some extent also in meat and meat products (Thijssen, H. H., et al., 1996, Phylloquinone and menaquinone-4 distribution in rats: synthesis rather than uptake determines menaquinone-4 organ concentrations, J Nutr 126:537-43). Vitamin K2 is found in animals as menaquinone. It is the human activated form of vitamin K and is said to promote the healing of bone fractures. It is essential for the carboxylation of glutamate residues in many calcium-binding proteins such as calbindin and osteocalcin. These proteins are involved in calcium uptake and bone mineralization.

There is an established daily dosage for vitamin K1, but not for vitamin K2. A typical therapeutic oral dose for vitamin K2 for osteoporosis is 45 mg/day. Unlike for coagulation, a much higher level of vitamin K is needed for complete gamma-carboxylation of osteocalcin (Booth, S. L., and J. W. Suttie, 1998, J. Nutr 128:785-8). Vitamin K deficiency is associated with reduced hip bone mineral density and increased fracture risk in healthy elderly women. Animal studies have shown that the most potent form of vitamin K is vitamin K2, which was administered to rats at 0.1 mg/kg orally (Akiyama, Y., et al., Biochem Pharmacol 49:1801-7). Vitamin K2, in the form of menaquinone-4, is the most biologically active form. It has been extensively studied in the treatment of osteoporosis. In one of these studies, 241 osteoporotic women were given 45 mg/day vitamin K2 and 150 mg elemental calcium. After two years, vitamin K2 was shown to maintain lumbar bone mineral density, significant lower fracture incidence (10% versus 30% in the control group (Shiraki, M., et al., J Bone Miner Res 15:515-21).

Vitamin K2, but not vitamin K1, may inhibit the calcification of arterial plaque. In 1996, animal studies involving rats found high dose of vitamin K2 (100 mg/kg body weight daily) inhibited the increase in calcium in both kidneys and aorta induced by megadose of synthetic vitamin D (Seyama, Y., et al., Int J Vitam Nutr Res 66:36-8). A similar study was conducted with rabbits. High dose of Vitamin K2 (1-10 mg/kg daily for 10 weeks) inhibited the atherosclerotic plaque progression in the aorta and pulmonary arteries (Kawashima, H., Y. et al., 1997. Jpn J Pharmacol 75:135-43).

Vitamin K2 was also seen to reduce total cholesterol levels, lipid peroxidation, ester cholesterol deposition in the aorta and factor X activity in plasma compared to the control group. A study involving more than 500 postmenopausal women investigated the relation between vitamin K1 and vitamin K2 intake and coronary calcification. Sixty-two percent of the women sampled for the study had coronary calcification. Only vitamin K2 intake was associated with the trend toward decreasing coronary calcification (Beulens, J. W., et al., Atherosclerosis 203:489-93).

In some embodiments, a composition of the present disclosure comprises one or more of the vitamins or vitamers above. A composition may comprise one or more of the vitamins or vitamers above in amounts between 1 μg and 1,000 mg per dose.

In some embodiments, a composition of the present disclosure may further comprise antioxidant compounds. These may include, but are not limited to, nonenzymatic compounds such as tocopherol (aTCP), coenzyme Q10 (Q), cytochrome c (C) and glutathione (GSH), and enzymatic components such as manganese superoxide dismutase (MnSOD), catalase (Cat), glutathione peroxidase (GPX), phospholipid hydroperoxide glutathione peroxidase (PGPX), glutathione reductase (GR); peroxiredoxins (PRX3/5), glutaredoxin (GRX2), thioredoxin (TRX2) and thioredoxin reductase (TRXR2). A composition may comprise one or more of the antioxidant compounds above in amounts between 1 μg and 1,000 mg per dose.

In some embodiments, a composition of the present disclosure may further comprise a stabilized oxidative species. The stabilized oxidative species may be, without limitation, one or more of H₂O, O₂, H₂O₂, Cl₂O, and H₃O.

Other adjuncts may include selenium and/or selenocysteine at concentrations of 60 to 90 μg per dose. Other adjuncts may also include other trace elements and their salts, including, but not limited to, copper, chromium, iodine, fluoride, zinc, manganese, molybdenum, and iron.

In some embodiments, compositions of the present disclosure may be formulated by combining pharmaceutical grade compounds into a stable therapeutic composition. Compounds may be added in desired amounts to a vessel, with water added to complete a final volume. In some embodiments, a composition of the present disclosure comprises a final volume of between 5 mL and 500 mL. In other embodiments, a composition comprises a final volume of about 250 mL. In some embodiments, the composition may be provided in 20 mL vials. A composition of the present invention may be further diluted prior to administration. For example, a 20 mL vial may be diluted with saline to a 100 mL dispensed volume for administration. In other embodiments, the liquid formulation may be reduced to dry solid via lyophilization. The lyophilized formulation may then be reconstituted to a particular volume prior to administration.

Table 1 shows various formulations of the composition according to exemplary embodiments of the present disclosure per 20 mL vial:

TABLE 1
Exemplary formulations for the disclosed compositions

| Component | mg/dose | mg/dose | mg/dose | mg/dose | mg/dose | mg/dose | mg/dose | mg/dose |
|---|---|---|---|---|---|---|---|---|
| L-Ascorbic | 0 | 450 | 900 | 900 | 12 | 2,000 | 2,000 | 0 |
| Acid USP | | | | | | | | |
| Dehydroascorbic | 0 | 0 | 0 | 12 | 900 | 2,000 | 2,000 | 4,000 |
| Acid | | | | | | | | |
| Thiamine HCl | 63.33 | 63.33 | 63.33 | 63.33 | 63.33 | 63.33 | 63.33 | 63.33 |
| USP | | | | | | | | |
| Magnesium | 808 | 808 | 808 | 808 | 808 | 808 | 808 | 808 |
| Sulfate USP | | | | | | | | |
| Cyanocobalamin | 1.93 | 1.93 | 1.93 | 1.93 | 1.93 | 1.93 | 1.93 | 1.93 |
| USP | | | | | | | | |
| Niacinamide | 119 | 119 | 119 | 119 | 119 | 119 | 119 | 119 |
| USP | | | | | | | | |
| Pyridoxine | 119 | 119 | 119 | 119 | 119 | 119 | 119 | 119 |
| HCl USP | | | | | | | | |
| Riboflavin | 2.53 | 2.53 | 2.53 | 2.53 | 2.53 | 2.53 | 2.53 | 2.53 |
| 5′Phosphate | | | | | | | | |
| USP | | | | | | | | |
| Calcium D- | 2.93 | 2.93 | 2.93 | 2.93 | 2.93 | 2.93 | 2.93 | 2.93 |
| Pantothenate | | | | | | | | |
| USP | | | | | | | | |
| Sodium | 840 | 840 | 840 | 840 | 840 | 3,360 | 3,360 | 3,360 |
| Bicarbonate | | | | | | | | |
| USP | | | | | | | | |
| WFI (water | | | | | | | | |
| for injection) | balance | balance | balance | balance | balance | balance | balance | balance |
| | mM/dose | mM/dose | mM/dose | mM/dose | mM/dose | mM/dose | mM/dose | mM/dose |
| HCl USP | 250 | 125 | 0 | 6.5 | 6.5 | 0 | 250 | 0 |
| diluted with | | | | | | | | |
| WFI (mM @ | | | | | | | | |
| 20 ml) | | | | | | | | |

In some embodiments, the components of the compositions in Table 1 may be varied from the listed values by plus or minus 1%, 2%, 5%, or 10% according to therapeutic needs. The compositions of Table 1 may also further comprise additional components, as described above, according to therapeutic needs.

In some embodiments, compositions of the present disclosure may be stabilized to enhance shelf life. The compositions may be stabilized by suitable techniques as known to those of ordinary skill in the art, including, but not limited to, freezing, lyophilization, use of UV or spectral blocking vials (e.g., amber vials), overfilling with stabilizing gases such as nitrogen, bubbling a stabilizing gas through the solution, separating reactive species into multiple vials to be combined upon use, and cold chain storage. As one non-limiting example, the acid and buffer components of a composition may be separated into two vials. Other components of compositions of the present disclosure (e.g., cyanocobalamin, calcium d-pantothenate, and/or others) may be included in these vials or further separated into additional vials.

In some embodiments, the composition can be provided in a kit comprising (a) a first vial containing a stable therapeutic composition comprising a buffer solution comprising at least one pharmaceutical grade acid and at least one pharmaceutical grade pH buffering agent, wherein the buffer solution is sufficient to reduce the physiological bloodstream pH of a subject by 0.1 to 1.1, and wherein the buffer solution has a buffer capacity sufficient to sustain the reduction of the physiological bloodstream pH of the subject for between 1 minute and 1 week; and optionally (b) instructions for use.

In some embodiments, the composition can be provided in a kit comprising (a) a first vial containing an intravenous buffer solution comprising at least one pharmaceutical grade acid in a sterile aqueous solution; (b) a second vial containing at least one pharmaceutical grade pH buffering agent in a sterile aqueous solution, wherein, when combined, the contents of the two vials form an intravenous buffer solution, wherein the concentration of the pharmaceutical grade acid and the pharmaceutical grade pH buffering agent in the buffer solution is sufficient to provide a total titratable acid content of from 60 mmol/L to 3000 mmol/L when administered to a subject, and wherein the selection of the pharmaceutical grade acid and the pharmaceutical grade pH buffering agent is effective to provide a buffer solution pH of between 4 and 7.7; and optionally (c) instructions for use.

The vial can be an injection vial with a membrane that is suitable for inserting a syringe to pull the solution from the vial or a soft I.V. infusion bag. The composition can be contained in the vial in a sterile aqueous solution. The solution can be provided as a concentrated solution to which a diluent is added prior to administration. The diluent can be sterile water. The kit may further comprise a pre-filled container which contains the diluent. In a preferred embodiment, a soft infusion bag is pre-filled with diluent. Alternatively, the composition vial can contain a solution that is at a concentration that is suitable for injection without any dilution. Preferably, the solution for injection is isotonic. That is, the solution can contain salt, carbohydrates, such as glucose, NaHCO₃ or amino acids, such as glycine, and is isotonic with blood plasma. In other instances, the solution may be hypotonic so as to promote more rapid intracellular uptake or hypertonic so as to promote slower intracellular uptake.

In some embodiments, the kit contains two vials. The first vial contains at least one pharmaceutical grade acid in a sterile aqueous solution. For example, the first vial may contain pharmaceutical grade ascorbic acid, thiamine HCl, magnesium sulfate, cyanocobalamin, niacinamide, pyroxidine HCl, riboflavin 5′ phosphate, calcium D-pantothenate, and an aqueous solvent containing sodium chloride and water (for injection). The second vial contains at least one pharmaceutical grade pH buffering agent in a sterile aqueous solution. For example, the second vial may contain pharmaceutical grade sodium bicarbonate and an aqueous solvent containing sodium chloride and water (for injection). The contents of the vials may be stored under refrigeration or under freezing conditions.

In another embodiment, the kit may contain a container of a lyophilized powder that may be reconstituted prior to administration. The lyophilized powder may be an isotonic solution.

Each kit described herein may further comprise instructions for use. The instructions will, of course, depend upon the kit itself and whether a diluent is to be used or other components to be admixed with the pharmaceutical grade buffer solution prior to administration.

### III. Methods of Treatment

In one aspect, this disclosure provides a method for preventing, alleviating, or treating a hypoxia-related disease or condition, comprising administering an effective amount of a composition to a subject in need thereof to improve oxygen transport and thereby elevate blood oxygen levels, wherein the composition comprises: at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and wherein the selection of the pharmaceutical grade acid and the pharmaceutical grade pH buffering agent is effective to provide a buffer solution pH of between 4.0 and 7.7.

In some embodiments, the hypoxia-related disease or condition is cancer, angiogenesis, or an angiogenesis-related disorder. In some embodiments, the cancer is a tumor or a solid tumor. Cancer can be any one of breast cancer, pancreatic cancer, ovarian cancer, colon cancer, lung cancer, non-small cell lung cancer, in situ carcinoma (ISC), squamous cell carcinoma (SCC), thyroid cancer, cervical cancer, uterine cancer, prostate cancer, testicular cancer, brain cancer, bladder cancer, stomach cancer, hepatoma, melanoma, glioma, retinoblastoma, mesothelioma, myeloma, lymphoma, and leukemia.

In some embodiments, the composition increases intracellular HCO₃⁻ level and thereby promotes hemoglobin affinity for oxygen. The representative formulations and dosage of the composition are described in Table 1.

In some embodiments, the subject suffers a blood electrolyte imbalance, which is a result of excess acid or bicarbonate.

In some embodiments, the method comprises elevating pO₂ level in the venous blood in the subject using the described composition. The representative formulations and dosage of the composition are described in Table 1.

In another aspect, this disclosure also provides a method for treating a subject suffering from a condition characterized by elevated serum calcium. The method comprises administering an effective amount of a composition to the subject to reduce blood calcium levels, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for restoring tumor suppressor protein p53 function in a subject. The method comprises administering an effective amount of a composition to the subject, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for suppressing tumor aggression in a subject having a cancer while restoring angiogenesis in healthy tissue of the subject. The method comprises administering an effective amount of a composition to the subject to increase eNOS and suppress iNOS, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for treating a subject having a cancer and suffering from elevated blood glucose related to the cancer. The method comprises administering an effective amount of a composition to the subject to improve pituitary, thyroid and renal function, thereby reducing blood glucose levels, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In some embodiments, the composition reduces cortisol levels, thereby reducing circulating glucose by relieving mitochondrial stress and endoplasmic reticulum stress. The representative formulations and dosage of the composition are described in Table 1.

In another aspect, this disclosure also provides a method for inhibiting poly ADP ribose polymerase (PARP). The method comprises administering to a subject in need thereof an effective amount of a composition, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In another aspect, this disclosure also provides a method for restoring a disturbed bone marrow microenvironment. The method comprises administering an effective amount of a composition to a subject in need thereof, the method comprising at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In yet another aspect, this disclosure also provides a method for promoting apoptosis in cancer. The method comprises administering an effective amount of a composition to a subject in need thereof, thereby eliciting a temporarily elevated acidic pH in the bloodstream to further decreasing intracellular pH which results in acidic stress and apoptosis in cancer cells, wherein the composition comprises at least one pharmaceutical grade acid and at least one pharmaceutical grade pH buffering agent in a sterile aqueous solution, wherein the concentration of the pharmaceutical grade acid and the pharmaceutical grade pH buffering agent in the buffer solution is sufficient to provide a total titratable acid content between 60 mmol/L and 3000 mmol/L when administered to a subject, and wherein the selection of the pharmaceutical grade acid and the pharmaceutical grade pH buffering agent is effective to provide a buffer solution pH of between 4.0 and 7.7.

In another aspect, the present disclosure provides a method of modifying the metabolism of a subject, the method comprising administering to the subject a stable therapeutic composition comprising a buffer solution comprising at least one pharmaceutical grade acid and at least one pharmaceutical grade pH buffering agent, wherein the buffer solution is sufficient to reduce the physiological bloodstream pH of a subject by 0.01 to 1.1, and wherein the buffer solution has a buffer capacity sufficient to sustain the reduction of the physiological bloodstream pH of the subject for between 1 minute and 1 week.

Routes of administration for a therapeutically effective amount of a composition of the present disclosure include, but are not limited to, intravenous, intramuscular, or parenteral administration, oral administration, otic administration, topical administration, inhalation or otherwise nebulized administration, transmucosal administration and transdermal administration. Compositions of the present disclosure may also be formulated for intravenous, bolus, dermal, oral, otic, suppository, buccal, ocular, or inhalation delivery. For intravenous or parenteral administration, i.e., injection or infusion, the composition may also contain suitable pharmaceutical diluents and carriers, such as water, saline, dextrose solutions, fructose solutions, ethanol, or oils of animal, vegetative, or synthetic origin. It may also contain preservatives, and buffers as are known in the art. When a therapeutically effective amount is administered by intravenous, cutaneous or subcutaneous injection, the solution can also contain components to adjust pH, tonicity, stability, and the like, all of which is within the skill in the art. For topical administration, the composition may be formulated in, e.g., liquid, gel, paste, or cream. In some embodiments, the composition may be administered via a topical patch. For ocular administration, the composition may be formulated in, e.g., liquid eye drops, or as a gel, paste, or cream to be applied to the surface of the eye and/or surrounding tissue. For otic administration, the composition may be formulated in, e.g., ear drops.

Compositions can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the agents can be formulated in liquid solutions, preferably in physiologically compatible buffers such as Hank's solution or Ringer's solution. In addition, the agents may be formulated in solid form and redissolved or suspended immediately prior to use. Lyophilized forms are also included.

A composition for intravenous, cutaneous, or subcutaneous injection may contain, in addition to peptide an isotonic vehicle such as Sodium Chloride Injection, Ringer's Injection, Dextrose Injection, Dextrose and Sodium Chloride Injection, Lactated Ringer's Injection Citrate Buffer pH 5.5, or other carriers, diluents, and additives as known in the art. As described fully herein, the pharmaceutical composition of the present invention may also contain stabilizers, preservatives, buffers, antioxidants, or other additive known to those of skill in the art. The pharmaceutical compositions are formulated for intravenous or parenteral administration. Typically, compositions for intravenous or parenteral administration comprise a suitable sterile solvent, which may be an isotonic aqueous buffer or pharmaceutically acceptable organic solvent.

Systemic formulations include those designed for administration by injection, e.g., subcutaneous, intravenous, intramuscular, intrathecal or intraperitoneal injection. Useful injectable preparations include sterile suspensions, solutions or emulsions of the active compound(s) in aqueous or oily vehicles. The compositions also can contain solubilizing agents, formulating agents, such as suspending, stabilizing and/or dispersing agent. The formulations for injection can be presented in unit dosage form, e.g., in ampules or in multidose containers, and can contain added preservatives. For prophylactic administration, the compound can be administered to a patient at risk of developing one of the previously described conditions or diseases. Alternatively, prophylactic administration can be applied to avoid the onset of symptoms in a patient suffering from or formally diagnosed with the underlying condition.

Formulations can comprise other ingredients for the treatment of the organism as a whole. For example, an anti-oxidant additive and/or pro-oxidant additive can be present. The latter may be an agent that acts as a preventive, while the former may be an agent that acts to treat a specific medical condition.

In addition to the formulations described above, pharmaceutical compositions may also be formulated as a depot preparation. Such long-acting formulations may be administered by implantation (for example subcutaneously or intramuscularly) or by intramuscular injection. Thus, for example, the agents may be formulated with suitable polymeric or hydrophobic materials (for example as an emulsion in an acceptable oil) or ion exchange resins, or as sparingly soluble derivatives, for example, as a sparingly soluble salt. Controlled release formula also includes patches, e.g., transdermal patches. Patches may be used with a sonic applicator that deploys ultrasound in a unique combination of waveforms to introduce drug molecules through the skin that normally could not be effectively delivered transdermally.

Various additives, known to those skilled in the art, may be included in formulations. Examples of additives include, but are not limited to, solubilizers, skin permeation enhancers, opacifiers, preservatives (e.g., anti-oxidants), gelling agents, buffering agents, surfactants (particularly nonionic and amphoteric surfactants), emulsifiers, emollients, thickening agents, stabilizers, humectants, colorants, fragrance, and the like. Inclusion of solubilizers and/or skin permeation enhancers is particularly preferred, along with emulsifiers, emollients, and preservatives. An optimum topical formulation comprises approximately: 2 wt. % to 60 wt. %, preferably 2 wt. % to 50 wt. %, solubilizer and/or skin permeation enhancer; 2 wt. % to 50 wt. %, preferably 2 wt. % to 20 wt. %, emulsifiers; 2 wt. % to 20 wt. % emollient; and 0.01 to 0.2 wt. % preservative, with the active agent and carrier (e.g., water) making of the remainder of the formulation. A skin permeation enhancer serves to facilitate passage of therapeutic levels of active agent to pass through a reasonably sized area of unbroken skin. Suitable enhancers are well known in the art and include, for example: lower alkanols such as methanol ethanol and 2-propanol; alkyl methyl sulfoxides such as dimethylsulfoxide (DMSO), decylmethylsulfoxide (C.sub.lO MSO) and tetradecylmethyl sulfoxide; pyrrolidones such as 2-pyrrolidone, N-methyl-2-pyrrolidone and N-(-hydroxyethyl)pyrrolidone; urea; N,N-diethyl-m-toluamide; C.sub.2-C. sub.6 alkane diols; miscellaneous solvents such as dimethylformamide (DMF), N,N-dimethylacetamide (DMA) and tetrahydrofurfuryl alcohol; and the 1-substituted azacycloheptan-2-ones, particularly 1-n-dodecylcyclazacycloheptan-2-one (laurocapram; available under the trademark AzoneR™ from Whitby Research Incorporated, Richmond, Va.).

Examples of solubilizers include, but are not limited to, the following: hydrophilic ethers such as diethylene glycol monoethyl ether (ethoxydiglycol, available commercially as Transcutol™) and diethylene glycol monoethyl ether oleate (available commercially as Softcutol™); polyethylene castor oil derivatives such as polyoxy 35 castor oil, polyoxy 40 hydrogenated castor oil, etc.; polyethylene glycol, particularly lower molecular weight polyethylene glycols such as PEG 300 and PEG 400, and polyethylene glycol derivatives such as PEG-8 caprylic/capric glycerides (available commercially as Labrasol™); alkyl methyl sulfoxides such as DMSO; pyrrolidones such as 2-pyrrolidone and N-methyl-2-pyrrolidone; and DMA. Many solubilizers can also act as absorption enhancers. A single solubilizer may be incorporated into the formulation, or a mixture of solubilizers may be incorporated therein. Suitable emulsifiers and co-emulsifiers include, without limitation, those emulsifiers and co-emulsifiers described with respect to microemulsion formulations. Emollients include, for example, propylene glycol, glycerol, isopropyl myristate, polypropylene glycol-2 (PPG-2) myristyl ether propionate, and the like.

Other active agents may also be included in formulations, e.g., anti-inflammatory agents, analgesics, antimicrobial agents, antifungal agents, antibiotics, vitamins, antioxidants, and sunblock agents commonly found in sunscreen formulations including, but not limited to, anthranilates, benzophenones (particularly benzophenone-3), camphor derivatives, cinnamates (e.g., octyl methoxycinnamate), dibenzoyl methanes (e.g., butyl methoxydibenzoyl methane), p-aminobenzoic acid (PABA) and derivatives thereof, and salicylates (e.g., octyl salicylate). In certain topical formulations, the active agent is present in an amount in the range of approximately 0.25 wt. % to 75 wt. % of the formulation, preferably in the range of approximately 0.25 wt. % to 30 wt. % of the formulation, more preferably in the range of approximately 0.5 wt. % to 15 wt. % of the formulation, and most preferably in the range of approximately 1.0 wt. % to 10 wt. % of the formulation. Topical skin treatment compositions can be packaged in a suitable container to suit its viscosity and intended use by the consumer. For example, a lotion or cream can be packaged in a bottle or a roll-ball applicator, or a propellant-driven aerosol device or a container fitted with a pump suitable for finger operation. When the composition is a cream, it can simply be stored in a non-deformable bottle or squeeze container, such as a tube or a lidded jar. The composition may also be included in capsules such as those described in U.S. Pat. No. 5,063,507. Accordingly, also provided are closed containers containing a cosmetically acceptable composition.

The amount of compound administered will depend upon a variety of factors, including, for example, the particular indication being treated, the mode of administration, whether the desired benefit is prophylactic or therapeutic, the severity of the indication being treated and the age and weight of the patient, the bioavailability of the particular active compound, and the like. Determination of an effective dosage is well within the capabilities of those skilled in the art coupled with the general and specific examples disclosed herein.

Dosages for a particular individual can be determined by one of ordinary skill in the art using conventional considerations, (e.g., by means of an appropriate, conventional pharmacological protocol). A physician may, for example, prescribe a relatively low dose at first, subsequently increasing the dose until an appropriate response is obtained. The dose administered to an individual is sufficient to effect a beneficial therapeutic response in the individual over time, or, e.g., to reduce symptoms, or other appropriate activity, depending on the application. The dose is determined by the efficacy of the particular formulation, and the activity, stability or serum half-life of the miRNA employed and the condition of the individual, as well as the body weight or surface area of the individual to be treated. The size of the dose is also determined by the existence, nature, and extent of any adverse side-effects that accompany the administration of a particular vector, formulation, or the like in a particular individual.

The duration of intravenous therapy using the pharmaceutical composition of the present invention will vary, depending on the condition being treated or ameliorated and the condition and potential idiosyncratic response of each individual mammal. The duration of each infusion is from <1 minute (e.g., bolus injection) to about 1 hour (intravenous delivery). The infusion can be repeated within 24 hours. Thus, a mammal can receive about 1 to about 25 infusions per day. Preferably, the number of infusions per day is 1 or 2. The period between each infusion can be 1, 2, 5, 10, 20, 30, 40, 50 minutes, or 1, 2, 3, 4, 5, 6, 7, 8, 9, 10, 11, 12 hours or more. The administration may also be administered at any of a variety of cadences, including hourly, daily, weekly, monthly, quarterly, bi-annually, annually, etc., or any other particular timeframe depending on the condition to be treated and/or the response of each individual mammal. In other embodiments, a pharmaceutical composition of the present invention may be administered as a single event, or may be administered over week-long, multi-week, month-long, year-long, or multi-year durations, or for any other desired duration as may be warranted.

Alternatively, the infusions can be given one after another without a substantial period in between. In one embodiment, the infusion lasts about 45 minutes. The dose may be repeated 2-3 times a week, depending on the severity of the relative or absolute deficits of nutrients in the patient. A clinical assessment may be necessary in order to establish the status, but can be limited to a review of medical history, subjective review of symptoms, the subjective opinion of the mammal when human or upon review of any specific deficits.

In another embodiment of administration, administration is alternated between two solutions: one acid shifting (AS) and one base shifting (BS) as described above. Alternating administration of AS/BS/AS/BS in various cadences would be expected to induce more pH swings from acidic towards basic or from basic towards acidic. Such events, as induced through exercise, are recognized for their value in promoting nitric oxide (NO) release for vasodilation (Capellini, Verena K., et al., 2013, The Effect of Extracellular pH Changes on Intracellular pH and Nitric Oxide Concentration in Endothelial and Smooth Muscle Cells from Rat Aorta, PLOS One, 8(5):e62887), and to promote cardiolipin repair and remodeling (Khalafat, Nada, et al., 2011, Lipid Packing Variations Induced by pH in Cardiolipin-Containing bilayers: The Driving Force for the Cristae-Like Shape Instability, Biochimica et Biophysica Acta—Biomembranes, 1808(11):2724-2733). These alternating administrations may each last between 0.5 and 60 minutes, and may be alternated one, two, or more times as necessary to achieve the desired therapeutic effect. The AS and BS administrations need not necessarily be identical in either their shifting effect or duration of administration. That is, for example, an AS composition may affect a larger shift over a shorter administration, while the BS composition may affect a smaller shift over a longer administration. In some embodiments, an exemplary administration profile may be a 5 minute AS administration followed by a 10 minute BS administration, repeated two times (i.e., 5/10/5/10). Other exemplary administration profiles may be, e.g., 10/10/10/10 or 0.5/0.5/0.5/0.5.

Efficacy of treatment may be determined by measuring biomarkers before, during, and/or after administration of a composition of the present disclosure, or before, during and/or after administration of a course of treatment using compositions of the present disclosure. Exemplary biomarkers, and the indications for which they may be used, are shown in Table 2, and may include, e.g., AlMicro, tubular disorders and electrolyte imbalance; A2Macro, cerebral small vessel disease, liver fibrosis; ACE, high blood pressure, heart failure, diabetic nephropathy; Adiponectin, vascular disease, metabolic syndromes; Apo A-I, high density lipid particles; Apo A-II, HDL metabolism; Apo C-II, ischemic stroke, heart disease; Apo C-III, metabolic syndrome and hypertriglyceridemia; Apo H, type 2 diabetes, metabolic syndrome; AT-III, venous thrombosis, abnormal coagulation; B2M, peripheral arterial disease; BDNF, psychiatric disorders; CD163, HIV infection, inflammation, cardiovascular disease; CD40, atherosclerotic instability; CD40-L, cellular proliferation; CgA, tumors; C-Peptide, metabolic syndrome; CRP, inflammation and tissue damage; Cystatin-C, cardiovascular disease, electrolyte imbalance; EGF, cellular proliferation; EN-RAGE, inflammation, heart disease; EPO, anemia, chronic kidney disease; E-Selectin, inflammation, electrolytic imbalances; Factor VII, thrombosis (blood clotting); Ficolin-3, diabetic peripheral neuropathy; FRTN, blood disorders, anemia; FSH, pregnancy complications; GDF-15, mitochondrial diseases; GLP-1 total, type 2 diabetes, insulin secretion; HB-EGF, epithelial cell proliferation (inflammation); ICAM-1, inflammation; IFN-gamma, inflammation and immune response; IL-1 alpha, inflammation; IL-1 beta, inflammation; IL-10, inflammation; IL-12p40, inflammation, multiple sclerosis, Alzheimer's disease; IL-12p70, peritonitis, inflammation; IL-15, Alzheimer's disease; IL-17, inflammation, lupus, cerebral vasculitis; IL-18, metabolic syndrome, acute kidney injury; IL-1ra, inflammation; IL-2, inflammation; IL-23, inflammation, lupus; IL-3, inflammation, cell growth, proliferation, and differentiation; IL-4, inflammation; IL-5, inflammatory factors, asthma, chronic obstructive pulmonary disease; IL-6, inflammation; IL-6r, coronary heart disease; IL-7, immune-mediated inflammatory diseases; IL-8, inflammation; IP-10, tuberculosis related complications; LH, infertility; Lp(a), cardiovascular diseases; MCP-1, inflammation; MCP-2, tuberculosis; MCP-4, asthma, metastasis; M-CSF, metabolic, hematologic and immunologic abnormalities; MIG, heart failure and left ventricular dysfunction; MIP-1 alpha, cytokine expression for high fat diet, wound healing; MIP-1 beta, autoimmune disorders; MIP-3 alpha, tissue injury in ischemic stroke and autoimmune diseases; MMP-3, ischemic and hemorrhagic stroke; MMP-9, ischemic and hemorrhagic stroke; MPIF-1, Kawasaki disease (inflammation in the walls of some blood vessels); MPO, inflammation and ischemia; Myoglobin, inflammation and ischemia; NAP-2, hepatitis B; NGF-betac, Alzheimer's disease, psychological disorders; Nr-CAM, Alzheimer's disease, cognitive disorders; Osteocalcin, osteoporosis, bone formation; PAI-1, metabolic syndrome; PARC, Gaucher disease (enlargement of liver/spleen); PDGF-BB, osteoblast development and bone formation, liver fibrosis; PEDF, cardiometabolic disorders; Periostin, asthma; PLGF, angiogenesis, vasculogenesis and lymphangiogenesis; PPP, endocrine pancreatic tumors; PRL; P-Selectin, inflammation; RAGE, chronic inflammatory diseases; RANTES, abdominal aortic aneurysm, viral diseases; Resistin, inflammation, cardiovascular disease; S100-B, brain damage and blood-brain barrier disruption; SAA, inflammation; SAP, acute and chronic inflammation; SCF, tumor proliferation; SHBG, thyroid disorders, pituitary diseases; SOD-1, amyotrophic lateral sclerosis; Sortilin, coronary artery disease, affective disorders; ST2, inflammation and adhesion; TAFI, arterial thrombosis, acute ischemia; TBG, thyroid related disorders; TIMP-1, tissue remodeling, wound healing and tumor metastasis; TN-C, myocarditis; TNF-alpha, inflammation; TNF-beta, inflammation, cardiovascular disease; TNFR2, ischemic stroke, insulin disorders; TTR, metabolic and septic disorders; VCAM-1, inflammation; VEGF, angiogenesis, hypoxia; Vitronectin, Alzheimer's disease; and vWF, arrhythmia, acute arterial damage.

TABLE 2
Biomarkers for determining treatment efficacy

| Tier II | Reference | Regulation during | Pathological |
|---|---|---|---|
| Biomarkers | Range | diseased state | relevance |
| E-Selectin | 30 pg/ml-18000 pg/ml* | Up | Inflammation |
| L-Selectin | 100 pg/ml-25 ng/ml | Up | Inflammation |
| P-Selectin | 20 pg/ml-30 ng/ml | Up | Inflammation |
| Intercellular Adhesion | 150 pg/ml-20 ng/ml | Up | Inflammation |
| Molecule-1 (ICAM-1) | | | |
| Vascular Cell Adhesion | 0.3 ng/ml-60 ng/ml | Up | Inflammation |
| Molecule-1 (VCAM-1) | | | |
| Epidermal Growth | 1 pg/ml-200 pg/ml | Up | Cellular |
| Factor (EGF) | | | Proliferation |
| Interferon-g (IFN-g) | 15.6-1,000 pg/mL | Up | Inflammation and |
| | | | Immune Response |
| Interleukin-1a (IL-1a) | 0.5 pg/ml-300 pg/ml | Up | Inflammation |
| Interleukin-lb (IL-1b) | 0.3 pg/ml-100 pg/ml | Up | Inflammation |
| Interleukin-2 (IL-2) | 4 pg/ml-1,500 pg/ml | Up | Inflammation |
| Interleukin-4 (IL-4) | 5 pg/ml-200 pg/ml | Up | Inflammation |
| Interleukin-6 (IL-6) | 3 pg/ml-1,000 pg/ml | Up | Inflammation |
| Interleukin-8 (IL-8) | 1 pg/ml-600 pg/ml | Up | Inflammation |
| Interleukin-10 (IL-10) | 1 pg/ml-150 pg/ml | Up | Inflammation |
| Monocyte Chemotactic | 2 pg/ml-500 pg/ml | Up | Inflammation |
| Protein-1 (MCP-1) | | | |
| Tumour Necrosis | 30 pg/ml-6,000 pg/ml | Up | Inflammation |
| Factor-a (TNF-a) | | | |
| Vascular Endothelial | 31-86 pg/mL | Up | Hypoxia |
| Growth Factor (VEGF) | | | |
| SAA | 0.5 ng/ml-300 ng/ml | Up | Inflammation |
| Fibrinogen | 150-400 mg/dL | Up | Thrombosis |
| C-Reactive | 0-10 mg/dL | Up | Inflammation and |
| Protein (CRP) | | | Tissue Damage |
| Apo A1 | Males: 94-176 mg/dL; | Up | Hight Density |
| | Females: 101-198 mg/dL | | Lipid Particles |
| Apo B | Male: 52-109 mg/dL; | Up | Low Density |
| | Female: 49-103 mg/dL | | Lipid Particles |
| Insulin | 4 μIU/ml-300 pIU/ml | Up | Metabolic Syndrome |
| Proinsulin | 0.313 ng/ml-20 ng/ml | Up | Metabolic Syndrome |
| C-peptide | 0.156 ng/ml-10 ng/ml | Up | Metabolic Syndrome |
| Myeloperoxidase | Adult Male = ≤50 mcg/L; | Up | Inflammation |
| | Adult Female = ≤30 mcg/L | | and schemia |
| CD40 Ligand | 32-2,000 pg/mL | Up | Cellular |
| | | | Proliferation |
| Bile Acid Panel | Varies | Varies | Cardiovascular |
| (16 bile acids) | | | Disease |
| p180 Kit (188 endogenous | Varies | Varies | Cardiometabolic |
| metabolites from 5 | | | Risk |
| compound classes) | | | |
| Oxidized LDL | 30-2,000 pg/mL | Up | Oxidative Stress |
| | | | and Low-Density |
| | | | Lipid Particle |
| ST2 | 0.156--10 ng/mL | Up | Inflammation and |
| | | | Adhesion |
| Creatine Kinase | 0-5.0 ng/mL | Up | Inflammation |
| Muscle Brain (CK-MB) | | | |
| Heart Type Fatty Acid | 102-25,000 pg/ml | Up | Inflammation and |
| Binding Protein (H-FABP) | | | Thrombosis |
| Myoglobin (Myo) | Adult Male = ≤50 mcg/L; | Up | Inflammation and |
| | Adult Female = ≤30 mcg/L | | Ischemia |
| Troponin I (cTnI) | ≤0.05 ng/mL | Up | Cardiovascular |
| | | | Disease |
| Adiponectin | 0.38-12 ng/mL | Up | Inflammation and |
| | (www.k-assay.com) | | Cardiac Disease |
| Cystatin C | 0.3 ng/ml-20 ng/ml | Up | Cardiovascular |
| | | | Disease |
| Catalase | 0.313 ng/ml-20 ng/ml | Up | Oxidative Stress |
| p53 | 3.1 U/ml-100 U/ml | Down | Apoptosis |

In some embodiments, the composition can be delivered by intravenous, intramuscular, or parenteral administration, oral administration, otic administration, topical administration, inhalation administration, transmucosal administration, and transdermal administration. In some embodiments, the intravenous administration is a bolus delivery. In some embodiments, the composition is administered by local delivery.

In some embodiments, the methods described above further comprise administering to the subject a second agent. The composition can be administered to the subject before or after administrating the second agent. In some embodiments, the composition is administered concurrently with the second agent.

In some embodiments, the second agent may include an anti-cancer agent, such as: Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ameluz (Aminolevulinic Acid), Amifostine, Aminolevulinic Acid, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avastin (Bevacizumab), Avelumab, Axicabtagene Ciloleucel, Axitinib, Azacitidine, Azedra (Iobenguane I 131), Bavencio (Avelumab), BEACOPP, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutamide, BiCNU (Carmustine), Binimetinib, Bleomycin, Blinatumomab, Blincyto (Blinatumomab), Bortezomib, Bosulif (Bosutinib), Bosutinib, Braftovi (Encorafenib), Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, CAPDX, Carac (Fluorouracil—Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib), Crizotinib, CVP, Cyclophosphamide, Cyramza (Ramucirumab), Cytarabine, Cytarabine Liposome, Cytosar-U (Cytarabine), Dabrafenib, Dacarbazine, Dacogen (Decitabine), Dacomitinib, Dactinomycin, Daratumumab, Darbepoetin Alfa, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), Durvalumab, Duvelisib, Efudex (Fluorouracil—Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enzalutamide, Epirubicin Hydrochloride, EPOCH, Epoetin Alfa, Epogen (Epoetin Alfa), Erbitux (Cetuximab), Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Firmagon (Degarelix), Fludarabine Phosphate, Fluoroplex (Fluorouracil—Topical), Fluorouracil Injection, Fluorouracil—Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, FU-LV, Fulvestrant, Fusilev (Leucovorin Calcium), Gamifant (Emapalumab-lzsg), Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idarubicin Hydrochloride, Idelalisib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), lobenguane I 131, Ipilimumab, Iressa (Gefitinib), Irinotecan Hydrochloride, Irinotecan Hydrochloride Liposome, Istodax (Romidepsin), Ivosidenib, Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Larotrectinib Sulfate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid), Libtayo (Cemiplimab-rwlc), LipoDox (Doxorubicin Hydrochloride Liposome), Lomustine, Lonsurf (Trifluridine and Tipiracil Hydrochloride), Lorbrena (Lorlatinib), Lorlatinib, Lumoxiti (Moxetumomab Pasudotox-tdfk), Lupron (Leuprolide Acetate), Lupron Depot (Leuprolide Acetate), Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate, Methylnaltrexone Bromide, Midostaurin, Mitomycin C, Mitoxantrone Hydrochloride, Mogamulizumab-kpkc, Moxetumomab Pasudotox-tdfk, Mozobil (Plerixafor), Mustargen (Mechlorethamine Hydrochloride), MVAC, Myleran (Busulfan), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Plerixafor, Pomalidomide, Pomalyst (Pomalidomide), Ponatinib Hydrochloride, Portrazza (Necitumumab), Poteligeo (Mogamulizumab-kpkc), Pralatrexate, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, Ravulizumab-cwvz, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Retacrit (Epoetin Alfa), Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sancuso (Granisetron), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagraxofusp-erzs, Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Tavalisse (Fostamatinib Disodium), Taxol (Paclitaxel), Taxotere (Docetaxel), Tecentriq (Atezolizumab), Temodar (Temozolomide), Temozolomide, Temsirolimus, Thalidomide, Thalomid (Thalidomide), Thioguanine, Thiotepa, Tibsovo (Ivosidenib), Tisagenlecleucel, Tocilizumab, Tolak (Fluorouracil—Topical), Topotecan Hydrochloride, Toremifene, Torisel (Temsirolimus), Totect (Dexrazoxane Hydrochloride), TPF, Trabectedin, Trametinib, Trastuzumab, Treanda (Bendamustine Hydrochloride), Trexall (Methotrexate), Trifluridine and Tipiracil Hydrochloride, Trisenox (Arsenic Trioxide), Tykerb (Lapatinib Ditosylate), Ultomiris (Ravulizumab-cwvz), Unituxin (Dinutuximab), Uridine Triacetate, VAC, Valrubicin, Valstar (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xeloda (Capecitabine), XELIRI, XELOX, Xgeva (Denosumab), Xofigo (Radium 223 Dichloride), Xospata (Gilteritinib Fumarate), Xtandi (Enzalutamide), Yervoy (Ipilimumab), Yescarta (Axicabtagene Ciloleucel), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), Zytiga (Abiraterone Acetate).

In some embodiments, the composition is administered after the subject is treated with adjuvant or neo-adjuvant chemotherapy. In some embodiments, the composition is administered between 1 and 90 days after the subject is treated with adjuvant or neo-adjuvant chemotherapy.

In some embodiments, the methods described above further comprise administering to the subject a second dose of the composition. The second dose can be administered to the subject between 1 and 30 days after a first dose is administered.

### IV. Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous monkey, chimpanzee, etc) and a human). The subject may be a human or a non-human. In this context, a “normal,” “control,” or “reference” subject, patient or population is/are one(s) that exhibit(s) no detectable disease or disorder, respectively.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, “cancer” refers to a broad group of diseases characterized by the uncontrolled growth of abnormal cells in the body. Unregulated cell division may result in the formation of malignant tumors or cells that invade neighboring tissues and may metastasize to distant parts of the body through the lymphatic system or bloodstream.

“Apoptosis” refers to the process by which cells are programmed to die. Apoptosis is commonly triggered by cytochrome leakage from the mitochondria and accompanied by signaling cascades (caspases and other proteins) resulting in decreased mitochondrial and energy potential via the electron transport system, a build up of reactive oxygen species and free radicals and loss of membrane integrity.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “inhibits” or “blocks” are used interchangeably and encompass both partial and complete inhibition/blocking by at least about 50%, for example, at least about 60%, 70%, 80%, 90%, 95%, 99%, or 100%.

The terms “decrease,” “reduced,” “reduction,” “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

The term “effective amount,” “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

By way of example, an anti-cancer agent is a drug that slows cancer progression or promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, a prevention of impairment or disability due to the disease affliction, or otherwise amelioration of disease symptoms in the patient. Pharmacological effectiveness refers to the ability of the drug to promote cancer regression in the patient. Physiological safety refers to an acceptably low level of toxicity, or other adverse physiological effects at the cellular, organ and/or organism level (adverse effects) resulting from administration of the drug.

By way of example for the treatment of tumors, a therapeutically effective amount or dosage of the drug preferably inhibits cell growth or tumor growth by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits cell growth or tumor growth, i.e., preferably inhibits cell growth or tumor growth by 100%. The ability of a compound to inhibit tumor growth can be evaluated using the assays described infra. Inhibition of tumor growth may not be immediate after treatment, and may only occur after a period of time or after repeated administration. Alternatively, this property of a composition can be evaluated by examining the ability of the compound to inhibit cell growth. Such inhibition can be measured in vitro by assays known to the skilled practitioner. In other preferred embodiments described herein, tumor regression may be observed and may continue for a period of at least about 20 days, more preferably at least about 40 days, or even more preferably at least about 60 days.

As used herein, “administering” refers to the physical introduction of a composition comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a composition described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent” which is a biologically, physiologically, or pharmacologically active substance (or substances) that acts locally or systemically in a subject.

The terms “therapeutic agent,” “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British Pharmacopeia (BP), National Formulary (NF), European Pharmacopoeia (EP), Japanese Pharmacopeia (JP), etc.). Pharmaceutical grade further incorporates suitability for administration by means including topical, ocular, parenteral, nasal, pulmonary tract, mucosal, vaginal, rectal, intravenous and the like.

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound.

“Combination” therapy, as used herein, unless otherwise clear from the context, is meant to encompass administration of two or more therapeutic agents in a coordinated fashion, and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on administration of another therapeutic agent. For example, one therapeutic agent may be administered only after a different therapeutic agent has been administered and allowed to act for a prescribed period of time. See, e.g., Kohrt et al. (2011) Blood 117:2423.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest such as antibodies. The sample may be any tissue sample from the subject. The sample may comprise protein from the subject.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein purification may not be necessary.

Methods well known in the art for collecting, handling and processing urine, blood, serum, and plasma, and other body fluids, can be used in the practice of the present disclosure, for instance, when the antibodies provided herein are employed as immunodiagnostic reagents, and/or in an immunoassay kit. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. It can be necessary or desired that a test sample, particularly whole blood, be treated prior to immunoassay as described herein, e.g., with a pretreatment reagent. Even in cases where pretreatment is not necessary, pretreatment optionally can be done for mere convenience (e.g., as part of a regimen on a commercial platform). The sample may be used directly as obtained from the subject or following a pretreatment to modify a characteristic of the sample. Pretreatment may include extraction, concentration, inactivation of interfering components, and/or the addition of reagents.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “I” means any one of the items, any combination of the items, or all of the items with which this term is associated.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### V. Examples

### Example 1

### Effects of the Inventive Composition in Post-Chemotherapy Recovery

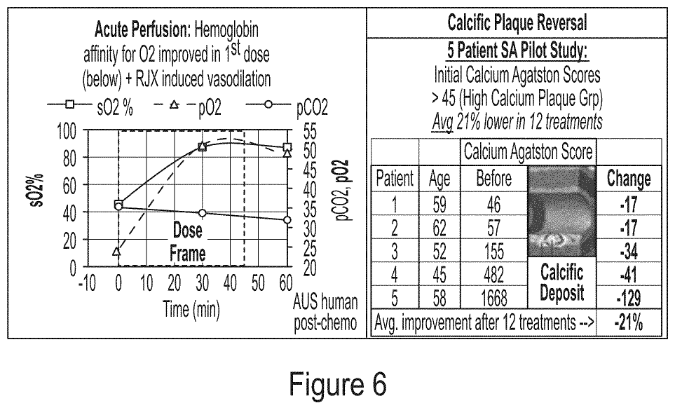

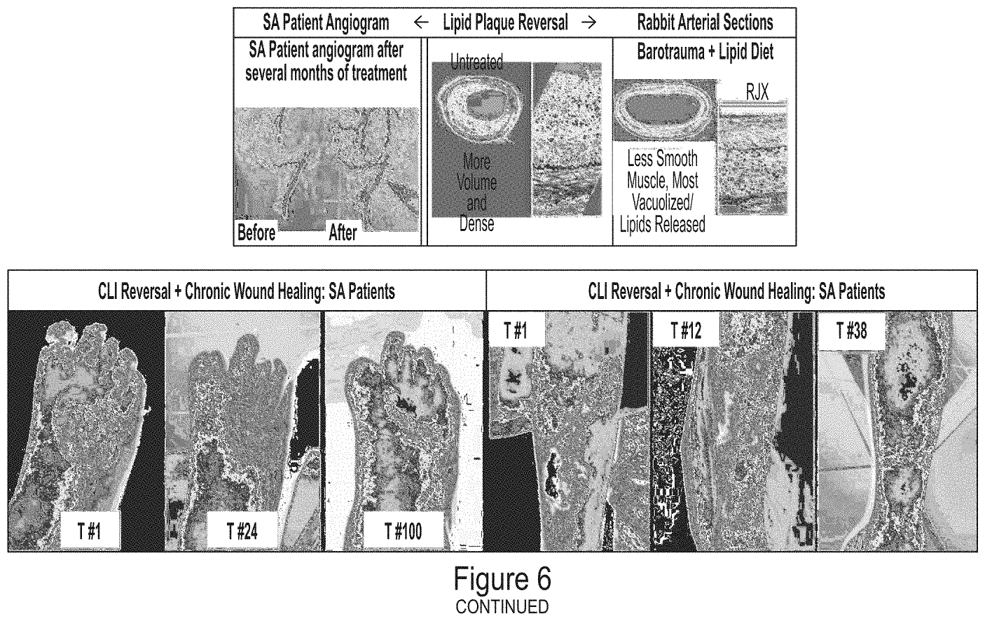

In this example, the inventive composition, as disclosed herein, was developed as an intravenous (IV) drug therapy with the potential to correct underlying perfusion deficits and correct intracellular electrolyte and metabolic aberrancies associated with critical limb ischemia (CLI) (FIG. 6).

The inventive composition (also referred to as “RJX” in this study) contains acid-base chemistry components that are able to cause an acidic bloodstream pH shift followed by an alkaline rebound, akin to aerobic exercise but in the absence of a coincident oxygen debt. These components may also have the potential to elicit metabolic advantage. The inventive composition also presents a concentration of magnesium to the bloodstream, which can work with the acid-base chemistry to rebalance “levels” of intracellular ionic species. The inventive composition also reduces intracellular calcium and resolution of intracellular acidosis, favorable for downregulating inflammatory signaling and hypertension and restoring nitric oxide function in nerves and arteries while suppressing the immune inducible form. The inventive composition also increases antioxidant and B vitamin metabolic chain resources, which work along with the electrolytic correction to reduce metabolic oxidative stress.

Research in the area of chemotherapy-related fatigue recognizes that this effect can “significantly disrupt normal functioning and quality of life for a substantial portion of cancer survivors, and may persist for years following cancer treatment.”¹ In fact, fatigue is believed to be the most widespread adverse side effect of cancer, affecting between 75-99% of patients². While the causes of persistent fatigue in such cases are not yet fully understood, evidence suggests that “chronic inflammation, autonomic imbalance, HPA-axis dysfunction, and/or mitochondrial damage, could contribute towards the disruption of normal neuronal function”.¹ Muscle loss with general atrophy, reduced numbers of mitochondria and increased oxidative stress are other commonly cited elements of post-chemotherapy fatigue². The diagnostic markers for cancer-related fatigue are presented in Table 1.1¹.

TABLE 1.1
Diagnostic markers for cancer-related fatigue

Symptoms present daily/nearly every day during the same 2-week
period in the past month:
Significant fatigue, diminished energy, or increased need
to rest, disproportionate to recent change in activity level
And at least five of the following symptoms:
Generalized weakness or limb heaviness
Diminished concentration or attention
Decreased motivation or interest to engage in usual activities
Insomnia or hypersomnia
Sleep is unrefreshing or non-restorative
Perceived need to struggle to overcome inactivity
Marked emotional reactivity (e.g., sadness, frustration, or
irritability) to feeling fatigued
Difficulty completing daily tasks attributed to feeling fatigued
Perceived problems with short-term memory
Post-exertional malaise lasting several hours

To reduce such fatigue, the literature recognizes exercise (Exerc Immunol Rev. 2016; 22: 82-93), metabolic chain supplementation including magnesium and B-vitamins (Nutrients. 2016 March; 8(3): 163), and antioxidant supplementation including ascorbic-acid. Recognizing that the composition as described utilizes acid-base components to stimulate an exercise-like bloodstream response, while delivering Magnesium, B-vitamins, and ascorbic acid (among other components), the composition as described is being studied for its potential to address post-chemotherapy fatigue.

### Post-Chemotherapy Patient Overview

This report relates to treatment of a 69-year-old Australian male with the composition as described, as authorized by the Australian Therapeutic Administration (agency similar to FDA) through a Category A special access process. The patient was treated with the composition as described after being treated for “Double-Hit” Non-Hodgkin's Lymphoma with R-hyper CVAD chemotherapy regimen, per table 1.2 below:

TABLE 1.2
Record of Chemotherapy Intervention, prior to
treatment with the composition as described

| | | | |
|---|---|---|---|
| Drug | Dose | Route | Day |

Hyper CVAD Part A

| | | | | |
|---|---|---|---|---|
| Dexamethasone | 40 | mg od | IV/PO | 1 to 4 and |
| | | | | 11 to 14 |
| Mesna | 600 | mg/m2 | IV infusion | 1 to 3 |
| Cyclophosphamide | 300 | mg/m2 bd | IV infusion | 1 to 3 |
| Methotrexate | 12 | mg | Intrathecal | 2 and 9 |
| Mesna | 800 | mg tds | PO | 4 |
| Doxorubicin | 50 | mg/m2 | IV infusion | 4 |
| Vincristine | 2 | mg | IV infusion | 4 and 11 |
| Pegfilgrastim | 6 | mg | Subcut | 5 |

Hyper CVAD Part B

| Methotrexate | 200 | mg/m2 | IV infusion | 1 |
|---|---|---|---|---|
| Methotrexate | 800 | mg/m2 | IV infusion | 1 |
| Calcium folinate | 15 mg/m2 every | | | 2 |
| (Leucovorin) | 6 hours * | | | |
| Cytarabine | 3,000 mg/m2 | | IV infusion | 2 and 3 |
| (Ara-C) | TWICE a day ** | | | |
| Pegfilgrastim | 6 | mg | Subcut | 4 |

Chemo rounds alternated between Regimen A and B

The regimen was as prescribed according to the eviQ treatment protocol hyper CVAD parts A and B for Non-Hodgkin lymphoma. eviQ is an online Australian Government evidence-based cancer treatment information website, accepted across Australia and increasingly across the world as a guide for best practice evidence-based cancer treatment protocols.

During and following his chemotherapy regimen, the patient presented with extreme fatigue. For example, it was reported that the patient “would have to pause and rest for tens of seconds at a time to simply complete a walk across the kitchen.” Additionally, impairments in cognitive engagement were reported as the patient was not able to engage in “sophisticated” conversations or communicate with “well chosen and clever” words in a manner consistent to his pre-chemotherapy status. A full list of impediments is presented in “Results Overview Section G” below, along with a symptomatic comparison to the recovered state following the treatment with the composition as described.

Accordingly, the therapy by the composition, as described, was proposed to expedite patient relief relative to such symptoms. the dosing of the composition commenced 16 days following the final chemotherapy treatment and was delivered at a target dose cadence of three times weekly (Monday, Wednesday, Friday), although some doses were spaced longer due to holidays and the Christmas break. (11 days break and 17 days break respectively), per table 1.3 below:

TABLE 1.3
Dose Cadence Record

| Dose | Day | Date |
|---|---|---|
| 1 | 0 | Nov. 28, 2018 |
| 2 | 2 | Nov. 30, 2018 |
| 3 | 5 | Dec. 3, 2018 |
| 4 | 7 | Dec. 5, 2018 |
| 5 | 9 | Dec. 7, 2018 |
| 6 | 12 | Dec. 10, 2018 |
| 7 | 23** | Dec. 21, 2018 |
| 8 | 40** | Jan. 7, 2019 |
| 9 | 42 | Jan. 9, 2019 |
| 10 | 44 | Jan. 11, 2019 |
| 11 | 47 | Jan. 14, 2019 |
| 12 | 49 | Jan. 16, 2019 |
| 13 | 51 | Jan. 18, 2019 |
| 14 | 54 | Jan. 21, 2019 |
| 15 | 56 | Jan. 23, 2019 |
| 16 | 58 | Jan. 25, 2019 |
| 17 | 61 | Jan. 28, 2019 |
| 18 | 63 | Jan. 30, 2019 |
| 19 | 65 | Feb. 1, 2019 |
| 20 | 68 | Feb. 4, 2019 |
| 21 | 70 | Feb. 6, 2019 |
| 22 | 72 | Feb. 8, 2019 |
| 23 | 75 | Feb. 11, 2019 |
| 24 | 77 | Feb. 13, 2019 |
| 25 | 79 | Feb. 15, 2019 |
| 26 | 82 | Feb. 18, 2019 |
| 27 | 84 | Feb. 20, 2019 |
| 28 | 86 | Feb. 22, 2019 |
| 29 | 89 | Feb. 25, 2019 |
| 30 | | |

**Holiday break and Christmas break of 11 days and 17 days, respectively.

The dose regimen consisted of a 20 ml dose of the composition as described diluted in 100 ml of saline and delivered via IV over a 45 minute period. As a monitoring element, venous blood gases were measured at T=0 min immediately prior to dosing, T=30 min during dosing, and following dosing at approximately T=60 min (within 15 minutes of end of infusion). Blood gas parameters included venous pH, HCO3-, sO2, pO2, TCO2, pCO2, K+, Ca+2, Glucose, Hematocrit % PCV, Hemoglobin concentration. Blood gasses were measured at every infusion for the first 8 infusions, thereafter once a week at each Monday infusion.

### Results Overview:

Specific responses, as described in the following sections, were noted regarding:
A) Relief of fatigue symptoms
B) Changes in blood attributes in 3 weeks
C) pH shifting as a result of drug administration
D) Stimulated exchange of electrolytes
E) Stimulated blood glucose response
F) Acute perfusion via enhancement of hemoglobin affinity for oxygen
G) Rapid red blood cell and hemoglobin recovery from depressed states
H) Acute vasodilatory response with progressing biasing towards a vasodilated state
I) Blood pressure and heartrate dose response similar to post-exercise phase response

### A) Relief of Fatigue Symptoms

From a clinical perspective—in other words, what was most meaningful for the patient aside from being declared in remission from his lymphoma—the turnaround in his symptoms of fatigue after he commenced infusions of the composition was the most significant. Important to note, after he completed his chemotherapy, he was physically and emotionally depleted, with no additional support from his clinician other than follow up PET scans. No relief for his post chemo fatigue was suggested nor offered, despite the negative life-altering impact.

Fatigue symptoms “as described” in the words of the patient's caregiver:
The patient exhibited “a total and complete fatigue and an end of energy. The fatigue would come on as sharp as a guillotine, and he had to stop moving immediately, and if holding an item, had to put it down or hand it over or he would drop it. When he lay down, he could feel a gradual re-enervation. His mind would still be completely alert, then would fall asleep within a minute.”
An equally severe and debilitating type of fatigue was mental fatigue and disorientation. The patient could not “consolidate or sequence his thoughts into an evolved stream of thinking or acting. He reports it as being clear in the moment, but couldn't string two moments together, yet could observe this state of mind—a surreal experience that lasted throughout his treatment.” When trying to carry a conversation, “his brain took a few seconds longer to catch up” under the weight of the physical fatigue.

TABLE 1.4
Patient Symptoms relative to diagnostic markers for cancer-related
fatigue (Patient's symptoms are bolded and in italics below)

Symptoms present daily or nearly every day during same 2-week
period prior to the composition as described:
Significant fatigue, diminished energy, or increased need
to rest, disproportionate to recent change in activity level
And at least five of the following symptoms:
Generalized weakness or limb heaviness
Diminished concentration or attention
Decreased motivation or interest to engage in usual activities
Insomnia or hypersomnia
Sleep is unrefreshing or non-restorative
Perceived need to struggle to overcome inactivity
Marked emotional reactivity (e.g., sadness, frustration,
or irritability) to feeling fatigued
Difficulty completing daily tasks attributed to feeling fatigued
Perceived problems with short-term memory
Post-exertional malaise lasting several hours

Below is a record of relief of fatigue symptoms “as described” in the words of the patient's caregiver. Notes on time to resolution listed where applicable.

Post Chemo: (remaining symptoms highlighted below)
1. Within the first few weeks, “Mental fatigue and disorientation” was observed to improve “significantly.
2. Over the first four weeks, “post exertional malaise lasting several hours persisted,” but since day 28, the patient's “tiredness would be what you would expect as normal relative to exertion levels.”
3. By day 85, “the generalized weakness and limb heaviness is nearly gone.” The patient “experienced a gradual and steady improvement which became obvious after the second week of treatment with RJX (note: the composition as described).” The patient “was a long-time regular attendee at the gym, even during breaks between chemotherapy, he would attend as much as he could manage, albeit with greatly reduced weights and intensity.” By day 65, “he was back to his full workout routine and full weights, as well as full normal activity.”
4. By day 85, sustained concentration largely restored, although “diminished concentration or attention still happens, but not every day and only for an hour or two at a time.”
5. By day 85, short term memory largely restored, although “short term memory lapses still happen on an incidental basis—occurring randomly (once every 2 or 3 days).”
6. By day 85, “decreased motivation (was) not (present) at all.”
7. By day 85. “Insomnia is improving significantly, as is his quality of sleep.”
8. By day 85, the patient exhibited an increase in engaged emotional activity, reported as “not feeling fatigued and more emotional to things whether good, bad or indifferent.”
   B) Changes in Blood Attributes after 9 and 85 Days

A comparison of blood attributes was made between draws preceding treatment (11/26 for most markers, 08/17 for select markers) with the composition as described and following administration of 4 doses in 9 days and 28 doses in 85 days. As summarized in Tables 1.5 (9 day results) and 6 (85 day results), healthy responses were observed for markers representing energy, immune function and inflammation, oxygen capacity, and various system-level effects. Energy markers suggested improved thyroid function (correlated to metabolic rate) in agreement with the patient experience of resolved fatigue. Various markers also indicated reductions in inflammation signaling, which can indicate reductions in future cancer risk and DVT clot risk among other things. Various markers also indicated progress to resolve anemia, which is key to ensuring adequate oxygen delivery. In addition, various markers suggest potential enhancement in liver, kidney function, along with potential improvements in endothelial function and potentially reduced risk of bone disorders. Although a slight elevation in PSA was observed at day 9, the values were still within age normal for the patient. Given that PSA scores can be influenced by exercise, it is possible that the PSA results were temporarily biased following the exercise-like stimulus of the composition as described (Kindermann W, et al. Urologe A. 2011; 50(2):188-196). By day 85, an overall reduction in total PSA was observed.

With the luxury of early patient data, such as featured in this report, Reven has designed a comprehensive monitoring plan for Phase II clinical work. At the same time, Reven is positioned to enter Phase II trials with mature anticipation of key modes that deliver healing in patients.

TABLE 1.5
Changes in blood attributes over the first 9 days of use of the composition as described.
Reven Compassionate Outreach: Post-Chemotherapy Patient
1st Dose 11/28, Results after 4 doses in 9 Days (% change relative to pre-RJX)

| | | | Pre- | Post | | |
|---|---|---|---|---|---|---|
| | | | RJX | RJX | | |
| | | | Draw dates unless | | | |
| | | | otherwise noted | | % Chg | Proposed Interpretation |
| | | Normal | 26 Nov. | 7 Dec. | 4 doses | 4 doses |
| Test Name | units | Range | 2018 | 2018 | Day 9 | Day 9 |

ENERGY

| | | | | | | |
|---|---|---|---|---|---|---|
| TSH | mIU/L | 0.5-4.0 | 4.8 | 4.1 | −15% | Hypothyroidism resolved: |
| | | | | | | Less |
| FT4 | pmol/L | 10.0-20.0 | 13 | 14 | 8% | thyroid stimulating hormone |
| | | | | | | to |
| FT3 | pmol/L | 3.5-6.0 | 3.7 | 4.2 | 14% | achieve higher-T4/T3 |
| | | | | | | metabolic rate |
| K⁺ | mmol/L | 3.6-5.4 | 4.8 | 4.4 | −8% | ATP pumps K+ into cells to |
| | | | | | | be ready |
| Ca²⁺ | mmol/L | 2.10-2.60 | 2.29 | 2.38 | 4% | Ca+2 from cytosol to |
| | | | | | | bloodstream |
| Serum | mmol/L | 3.4-5.4 | 6 | 4.9 | −18% | Glucose back in Control |
| Glucose | | | | | | |

SYSTEM LEVEL EFFECTS

| | | | | | | |
|---|---|---|---|---|---|---|
| Urea | mmol/L | 3.5-9.0 | 9.4 | 8.6 | −9% | Better liver (make less urea) |
| | | | | | | and/or better kidney (filter |
| | | | | | | urea) function |
| Creatinine | umol/L | 60-110 | 65 | 75 | 15% | Faster muscle mass recovery |
| Urate | mmol/L | 0.20-0.42 | 0.24 | 0.28 | 17% | Restore urate for antioxidant |
| | | | | | | role/to improve endothelial |
| | | | | | | function |
| Alk Phos | U/L | 35-110 | 89 | 65 | −27% | Reduced liver, bone disorder |
| PO4 | mmol/L | 0.75-1.5 | 1.24 | 1.6 | 29% | Tumor lysis? pH? Excess |
| | | | | | | Vit-D? |
| Total PSA** | ug/L | Free | 3.54 | 6.31 | 78% | PSA changes still within age |
| | | PSA/Total | | | | normal, |
| Free PSA** | ug/L | PSA: | 0.74 | 1.03 | 39% | Note: “exercise” can induce |
| | | Ratios | | | | temporary elevation effect |
| | | <10% | | | | |
| Free PSA | % | Abnormal | 21 | 16 | −24% | |
| Ratio** | | | | | | |

IMMUNE

| | | | | | | |
|---|---|---|---|---|---|---|
| WBC | ×10\hat{ }9/L | 4.0-11.0 | 8.1 | 3.4 | −58% | Fewer WBC's: quiesced |
| | | | | | | immune |
| Neut | ×10\hat{ }9/L | 2.0-7.5 | 3.9 | 2.1 | −46% | response --> less |
| | | | | | | inflammatory |
| Lymph | ×10\hat{ }9/L | 1.0-4.0 | 1.7 | 0.6 | −65% | signalling. Lower CRP/ |
| | | | | | | ESR: lower |
| Mono | ×10\hat{ }9/L | 0.2-1.0 | 1.5 | 0.7 | −53% | auto-immune (RA, lupus, |
| | | | | | | colitis, |
| CRP | mg/L | <6.0 | 15.7 | <4.0 | −75% | Crohn), heart attack, CAD, |
| | | | | | | cancer |
| ESR | mm/hr | <30 | 73 | 35 | −52% | More platelets for clotting |
| | | | | | | while |
| Platelets | ×10\hat{ }9/L | 150-400 | 95 | 296 | 212% | low ESR reduces DVT/clot |
| | | | | | | risk |
| Zinc** | umol/L | 10.0-18.0 | 9.9 | 11.1 | 12% | Less immune Zn |
| | | | | | | consumption |

OXYGEN CAPACITY

| | | | | | | |
|---|---|---|---|---|---|---|
| Reticulocytes | ×10\hat{ }9/1 | 20-100 | NA | 119 | — | Increase in young RBC's |
| RDW | % | 10.0-17.0 | 15.6 | 19.6 | 26% | New RBC's are bigger |
| Bilirubin | umol/L | <20 | 4 | 7 | 75% | Senescent RBC's cleared |
| Ferritin | ug/L | 30-400 | 576 | 225 | −61% | Iron used to make blood cells |
| Hb | g/L | 130-180 | 80 | 96 | 20% | Overall increase in Oxygen |
| | | | | | | capacity |
| Hct | | 0.40-0.54 | 0.25 | 0.29 | 16% | --> Anemia is resolving |

**Pre-RJX draw Aug. 17, 2018

While many Table 1.5 results bear discussion, an interpretation of WBC counts is given. Filgrastim was administered post-chemo/pre-administration of the composition as described to achieve mid-normal WBC counts. Then the composition as described possibly promoted an anti-inflammatory alkaline environment (complete metabolic chain towards alkaline+alkaline rebound) to potentially clear inflammatory macrophages. As such, it could work to reduce overall macrophage counts and possibly alter the ratio between pro-inflammatory (e.g., M1, CD16+) phenotypes and anti-inflammatory phenotypes (e.g., M2, CD16−). In the literature, it has been observed that WBC counts of 2.5<x<7.0 (E9/L) can be beneficial for NSLC cancer

TABLE 1.6
Changes in blood attributes over the first 85 days of use of the composition as described
Reven Compassionate Outreach: Post-Chemotherapy Patient
1st Dose 11/28, Results after 28 doses in 85 Days (% change relative to pre-RJX)

| | | | Pre- | Post | | |
|---|---|---|---|---|---|---|
| | | | RJX | RJX | | |
| | | | Draw dates unless | | | |
| | | | otherwise noted | | % Chg | Proposed Interpretation |
| | | Normal | 26 Nov. | 21 Dec. | 27 doses | 28 doses |
| Test Name | units | Range | 2018 | 2018 | Day 85 | Day 85 |

ENERGY

| | | | | | | |
|---|---|---|---|---|---|---|
| TSH | mIU/L | 0.5-4.0 | 4.8 | 4.6 | −4% | Hypothyroidism resolved: |
| | | | | | | Less |
| FT4 | pmol/L | 10.0-20.0 | 13 | 16 | 23% | thyroid stimulating |
| | | | | | | hormone to |
| FT3 | pmol/L | 3.5-6.0 | 3.7 | 4.6 | 24% | achieve higher-T4/T3 |
| | | | | | | metabolic rate |
| K⁺ | mmol/L | 3.6-5.4 | 4.8 | 4.8 | 0% | Mid normal |
| Ca²⁺ | mmol/L | 2.10-2.60 | 2.29 | 2.32 | 1% | Mid normal |
| Serum | mmol/L | 3.4-5.4 | 6 | 5.7 | −5% | Glucose back in Control |
| Glucose | | | | | | |

SYSTEM LEVEL EFFECTS

| | | | | | | |
|---|---|---|---|---|---|---|
| Urea | mmol/L | 3.5-9.0 | 9.4 | 8 | −15% | Better liver (make less urea) |
| | | | | | | and/or better kidney (filter |
| | | | | | | urea) function |
| Creatinine | umol/L | 60-110 | 65 | 75 | 15% | Faster muscle mass |
| | | | | | | recovery |
| Urate | mmol/L | 0.20-0.42 | 0.24 | 0.25 | 4% | Low normal |
| Alk Phos | U/L | 35-110 | 89 | 60 | −33% | Reduced liver, bone |
| | | | | | | disorder |
| PO₄ | mmol/L | 0.75-1.5 | 1.24 | 1.11 | −10% | Mid normal |
| Total PSA** | ug/L | Free | 3.54 | 2.61 | −26% | PSA measures improved |
| | | PSA/Total | | | | |
| Free PSA** | ug/L | PSA: Ratios | 0.74 | 0.49 | −34% | |
| | | <10% | | | | |
| Free PSA | % | Abnormal | 21 | 19 | −10% | |
| Ratio** | | | | | | |

IMMUNE

| | | | | | | |
|---|---|---|---|---|---|---|
| WBC | ×10\hat{ }9/L | 4.0-11.0 | 8.1 | 3.9 | −52% | Fewer WBC's: quiesced |
| | | | | | | immune |
| Neut | ×10\hat{ }9/L | 2.0-7.5 | 3.9 | 2.3 | −41% | Response -- > less |
| | | | | | | inflammatory |
| Lymph | ×10\hat{ }9/L | 1.0-4.0 | 1.7 | 0.6 | −65% | Signaling. Lower |
| | | | | | | CRP/ESR: lower |
| Mono | ×10\hat{ }9/L | 0.2-1.0 | 1.5 | 0.9 | −40% | Auto-immune (RA, lupus, |
| | | | | | | colitis, |
| CRP | mg/L | <6.0 | 15.7 | <4.0 | −75% | Chron), heart attack, CAD, |
| | | | | | | cancer |
| ESR | mm/hr | <30 | 73 | 10 | −86% | More platelets for clotting |
| Platelets | ×10\hat{ }9/L | 150-400 | 95 | 172 | 81% | While low ESR reduces |
| | | | | | | DVT/.clot risk |
| Zinc** | umol/L | 10.0-18.0 | 9.9 | NA | — | NA |

OXYGEN CAPACITY

| | | | | | | |
|---|---|---|---|---|---|---|
| Reticulocytes | ×10\hat{ }9/L | 20-100 | NA | NA | — | NA |
| RDW | % | 10.0-17.0 | 15.6 | 13.4 | 14% | Less senescent, more |
| | | | | | | homogeneous |
| Bilirubin | umol/L | <20 | 4 | 8 | 100% | Senescent RBC's cleared |
| Ferritin | ug/L | 30-400 | 576 | 102 | −82% | Iron used to make blood |
| | | | | | | cells |
| Hb | g/L | 130-180 | 80 | 134 | 68% | Anemia Resolved |
| Hct | | 0.40-0.54 | 0.25 | 0.4 | 60% | |

**Pre-RJX draw Aug. 17, 2018

Survivability (the described composition: 8.1→3.9) (Technol Cancer Res Treat. 2018; 17: 1533033818802813). For diffuse large-B cell lymphoma, survivability was improved when overall monocytes (the described composition: 1.5→0.9) and neutrophils (the described composition: 3.9→2.3) decreased, and when ratios of pro-inflammatory/anti-inflammatory monocytes (not measured) were lower (Oncotarget. 2017 Jul. 18; 8(29): 47790-47800).

### C) pH Shifting as a Result of Drug Administration

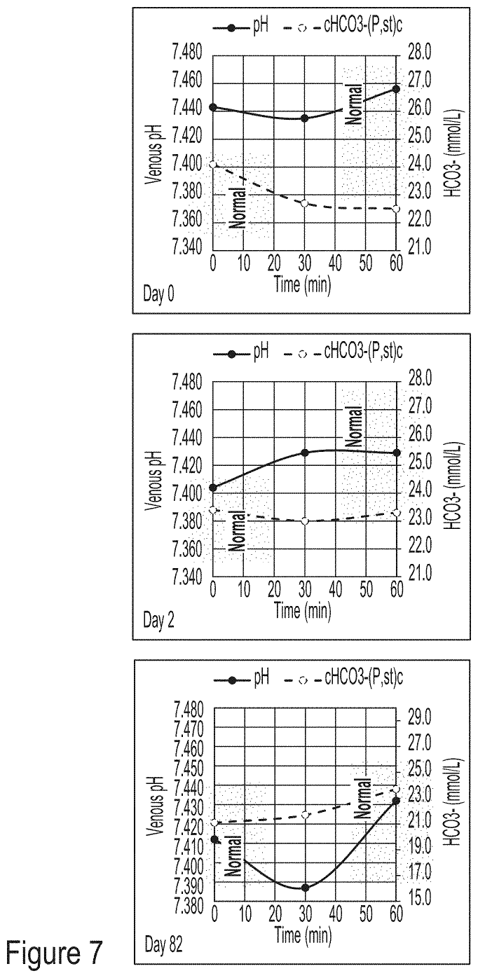

In response to dosing of the described composition, an observable exercise-like pH shift can be observed. As shown in the Day 0 response in FIG. 7, the pH response (black) can exhibit an acidic shift followed by an alkaline rebound, as renal and respiratory processes resolve the stimulus. In Day 2, no acid shifting was observed, potentially as an artifact of the coarse measurement cadence. Day 61 is also presented for longitudinal perspective. Alongside pH, HCO₃⁻ (gray dashed line) can respond in unison or move in the opposite direction. While the nature of these relationships is still under study, the signals are believed to represent a balance of a) HCO₃⁻ flow to/from the intracellular, b) HCO₃⁻ recycling/production from renal activity, c) and action of carbonic anhydrase to shift the equilibrium from respiratory burden CO₂ and H₂O states to HCO₃⁻ and H⁺ states.

### D) Stimulated Exchange of Electrolytes

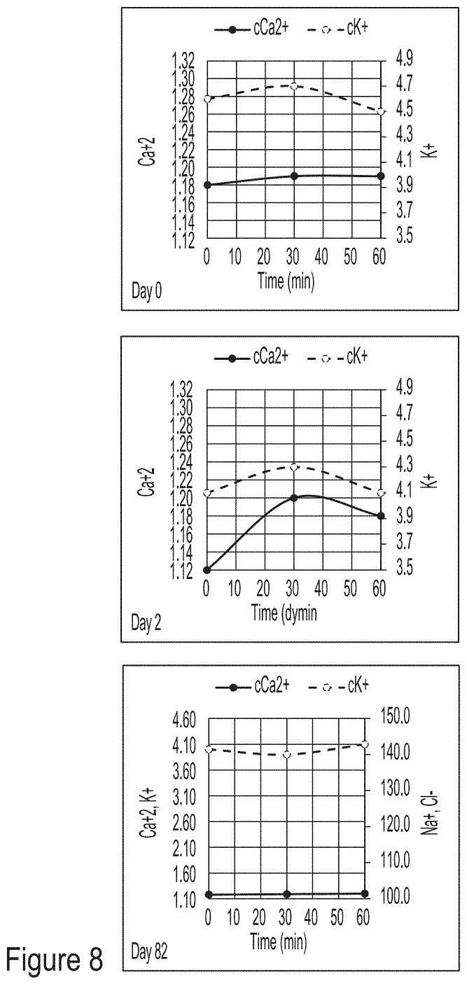

In response to dosing with the described composition, an observable electrolyte exchange can be observed. In general, Ca²⁺ was commonly seen to rise at certain points, perhaps as ATP assisted exchange processes were enabled to move Ca²⁺ from the cytosol to the bloodstream or as changes in acid status managed exchange to and from Ca²⁺ reservoirs, such as bone. K⁺ presentation was seen to reduce post-treatment, perhaps because ATP assisted processes were enabled to move K⁺ from the blood to the cytosol. Over sustained treatment, a distinct trend of lower bloodstream K⁺ can be noted (Day 0, 2 versus Day 82; FIG. 8). This possibly indicates that cells can better maintain intracellular K⁺ status (rarifying it from the blood) as desired for athletic output potential.

### E) Stimulated Blood Glucose Response

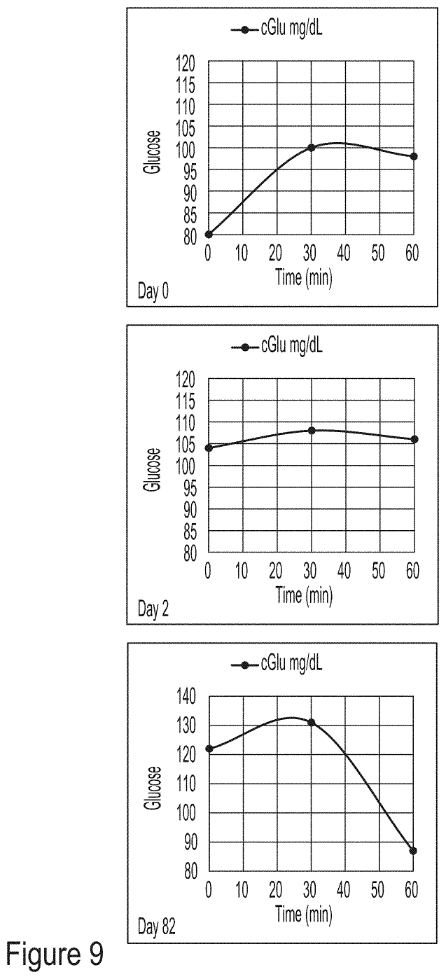

The described composition commonly stimulates an observable blood glucose response. As shown in FIG. 9, glucose levels were corrected during dose 1 from low normal of 80 mg/dl towards 100 mg/dl mid-normal levels. This could indicate a restoration of adrenal and pituitary function to better appropriate glucose for metabolic use. As a note, elevated blood glucose was measured in blood 2 days before dose 1 (Table 1.5) and measured at the low side of normal immediately before dose 1 (FIG. 9). High variability in blood glucose is one indication of impaired vagal function, which coordinates parasympathetic and sympathetic responses to maintain blood glucose, heart rate, and blood pressure. It is possible that the described composition helps restore the metabolic elements of vagal control and that the exercise-like stimulus of the described composition “trains” the requisite systems to better work together.

### F) Acute Perfusion Via Enhancement of Hemoglobin Affinity for Oxygen

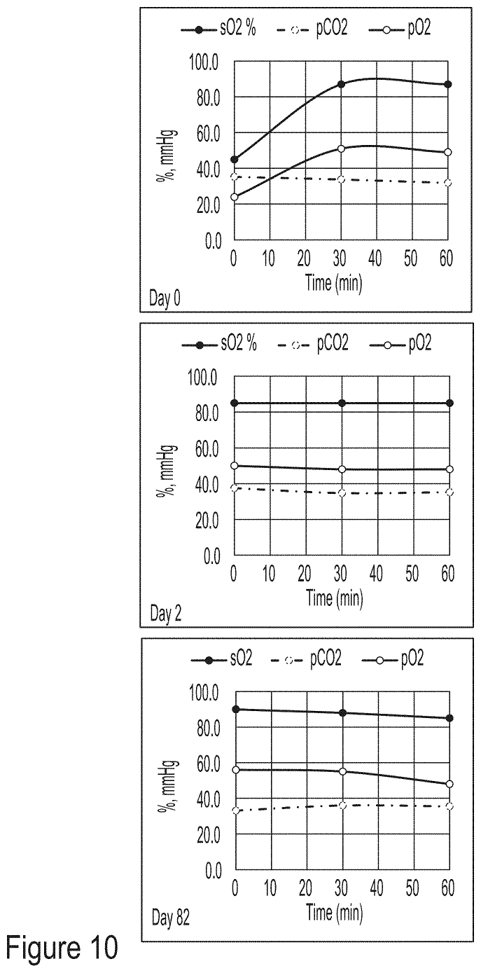

The described composition also induces changes in oxygen status (FIG. 10). As seen in the first dose response, blood oxygen (venous sO₂) was rapidly corrected from 45% to 87%. Furthermore, the sO₂ status remained >77% between doses, even when spaced a week apart. As one exception, the incoming sO₂ status fell to 60% after a holiday break of 20 days between doses 6 and 7. During dose 7 (not shown), sO₂ again corrected and was observed above 77% for all other TO. As a possible explanation, the described composition may increase intracellular HCO₃⁻ in red-blood cells to better absorb H⁺ so hemoglobin can carry O₂ instead. Also, the described composition may improve cell metabolism to make less H⁺.

G) Rapid Red Blood Cell and Hemoglobin Recovery from Depressed States

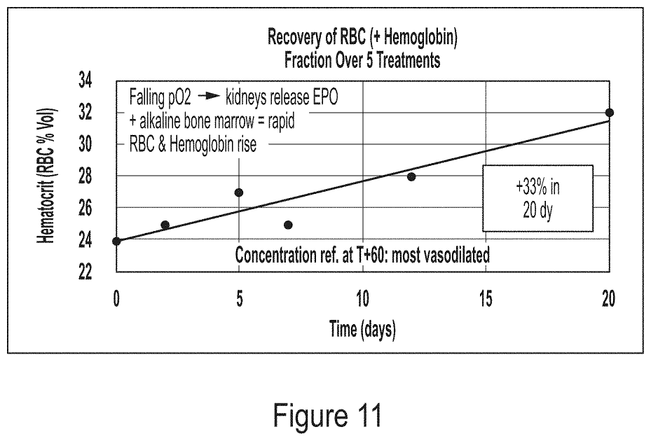

The patient exhibited an anemic state at the conclusion of his chemotherapy regimen. His hematocrit status was between 24% and 27% (based on TO and T60 Day 0 measurements), relative to a conventional low-normal status of 37%. During the course of therapy, a steady increase in hematocrit was observed (as introduced in the previous section). To establish a rate of recovery from anemia, the T60 (post dose-finish) hematocrit values for the first 6 treatments were plotted, as shown in FIG. 11. The T60 values were chosen as they represent the most vasodilated state, and ideally, represent a consistent level of vasodilation. As shown in FIG. 11, the first 5 doses were projected to invoke a 33% increase in RBC fraction as assessed over a 20-day span. It is also worth note that this recovery trajectory is on par with values cited for the current market leader, EPOGEN. Recognizing that the described composition can be administered daily, it is left to future studies to establish if the rate of response could have been improved with different administration strategies or a more dense dosing schedule.

This response is believed to be due in part to the falling pO₂ signal that is stimulated during the dosing (FIG. 11). Such a falling pO₂ would be expected to stimulate EPO (a naturally occurring hormone produced by cells in the kidneys that regulate the production of red blood cells in bone marrow) release from the kidneys to spur RBC growth in the bone marrow. The alkaline rebound element (FIG. 7) is a second element with potential to promote RBC production in bone marrow, as literature suggests that HCO₃⁻ status is linked to the erythrocyte response.

H) Acute Vasodilatory Response with Progressive Biasing Towards a Vasodilated State

As a consequence of the acidic bloodstream stimulus and magnesium and nicotinamide components in the described composition, it is believed that endothelin, prostacyclin, and NO-sGC vasodilatory pathways are promoted. This would drive an increase in vascular service volume, and require that fluids be pulled from the intracellular to maintain blood pressure, effectively promoting an anti-inflammatory process, such as is recognized in response to exercise. As a surrogate indication of vasodilation, acute changes in Hematocrit red blood cell fraction were studied. Given that material changes in hematocrit presentation occur on a timescale of days, not minutes, acute reductions in hematocrit concentration were interpreted to represent increases in plasma volume.

As shown in FIG. 12, a measurable vasodilation was observed as a consequence of dosing, as shown by a difference in starting vasodilation based on TO hematocrit concentration and final “100%” vasodilation based on the T60 hematocrit concentration. While the “100%” definition was chosen for convenience of plotting and to notionally communicate the principle, the absolute vasodilation cannot be numerically defended without radiotracer studies to characterize blood volume in both states, which was deemed impractical. Nonetheless, the data suggests that a substantial blood volume expansion (vasodilation) was observed. Moreover, the degree of vasodilation was observed to be highly variable in the first 6 doses and stable at TO for doses 7-10 with little additional dilation observed during treatment. Given that the patient reported first feeling extended periods of comfort at roughly this same time, it is believed that his vasculature was stabilizing into a pattern of being more nominally vasodilated, as his vasodilation control systems were restored to an elevated functional status through the therapy. The lower chart in FIG. 12B also indicates a progressive rise in hematocrit over the dosing cycle, which indicates a recovery of Red Blood cell status (discussed further in the next section).

### I) Blood Pressure and Heartrate Dose-Response Similar to Common Post-Exercise Response

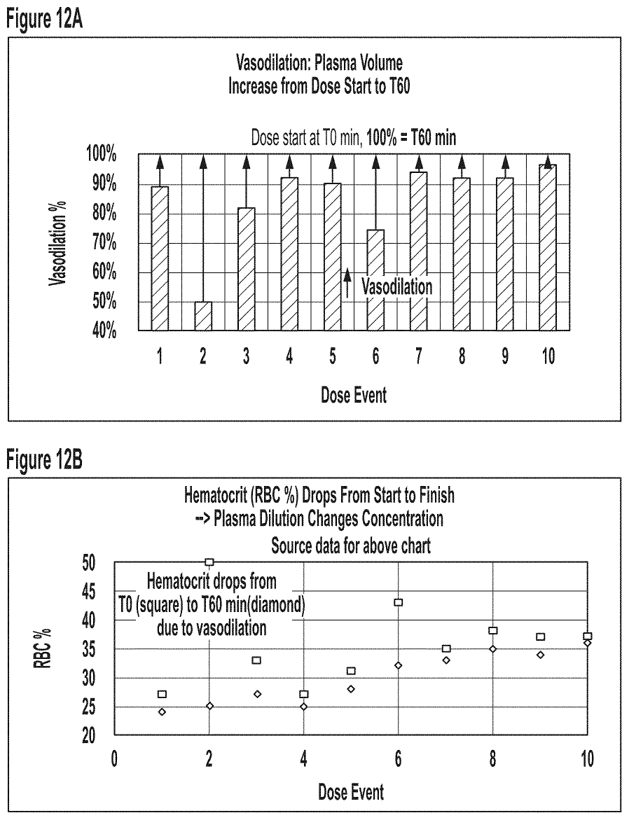

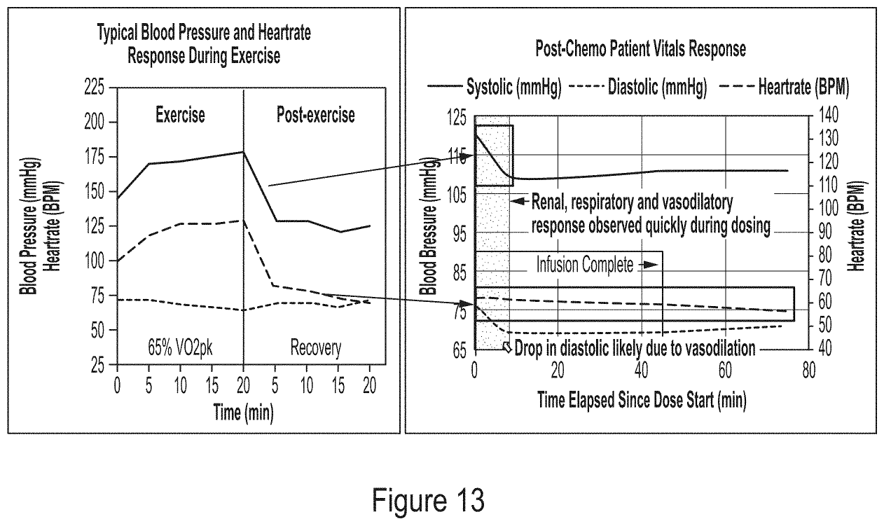

As another observation on the patient response to dosing, heart rate and blood pressure signals were observed to share several behavioral patterns with common post-exercise response.⁸ After dose initiation (FIG. 13, Dose 26 Day 82), systolic pressure was seen to drop in a frame of several minutes in a manner that is similar to a post-exercise response. The heartrate signal also showed a profile that steadily reduced over a 75 minute observation frame, again in a manner similar to the post-exercise exercise phase. It is possible that improved oxygenation from the described composition allows metabolic demands to be met more easily, so that heart rate and blood-pressure drop to maintain energy supply balance. In addition to these post-exercise like signals, a drop in diastolic was observed following dose start, likely indicating a vasodilation event, such as the responses are shown in FIG. 12.

8) Core Temperature Measurement During Submaximal Exercise: Esophageal, Rectal, and Intestinal Temperatures (Lee, Stuart & Jon Williams, W & M Schneider, Suzanne. (2000). Core Temperature Measurement During Submaximal Exercise: Esophageal, Rectal, and Intestinal Temperatures).

### Conclusion Summary:

The described composition was observed in a patient application to relieve and then completely dissipate post-chemotherapy fatigue. Described in this report are actions to:
A) Relief of Fatigue Symptoms: various life-changing symptoms were resolved to bring about a measurable improvement in wellness and wellbeing.
B) Changes in blood measurables following 6 doses over 21 days indicated potentially beneficial changes in health regarding energy, immune function and inflammation, oxygen capacity, and various system level effects.
C) Induce a pH shifting event with an alkaline rebound and elevated presentation of HCO3-, which is similar to that seen in the bloodstream during and after exercise.
D) Stimulate exchange of electrolytes in a manner consistent with exercise, and specifically showing a reduction in bloodstream potassium, potentially implying a higher maintenance of intracellular potassium.
E) Stimulate blood glucose response in a manner consistent with exercise, which showed an increase from low normal to mid-normal during the very first dose, and which was sustained throughout the 80 day observation period.
F) Acute perfusion via enhancement of hemoglobin affinity for oxygen, and specifically showing resolution of impaired oxygen delivery from 45% to 87% sO2 during the first dose, which was durably observed thereafter.
G) Rapid red blood cell and hemoglobin recovery from depressed states, and specifically showing a 33% increase by treatment day 20 following the first 5 doses.
H) Acute vasodilatory response observed, which progressed towards a sustained “predominantly vasodilated” state over successive treatments.
I) Blood pressure and heart rate were observed to drop during dosing in a manner similar to post-exercise response.

In this case of applying the described composition for fatigue relief in a post-chemotherapy patient, the described composition was observed to impart an effect from the very first dose with progressive improvement supported throughout the treatment course. The life-changing impact of this treatment is best summed up in the words of the patient's caregiver on day 89:
“There has been a huge improvement in mind, body and intention.
He is exuberant, focused, super-sharp, clear and alert at the high end of normal! Nothing dragging—functions are all back to being totally integrated.
He moved more fully into this state every time he had RJX (note: the described composition).”

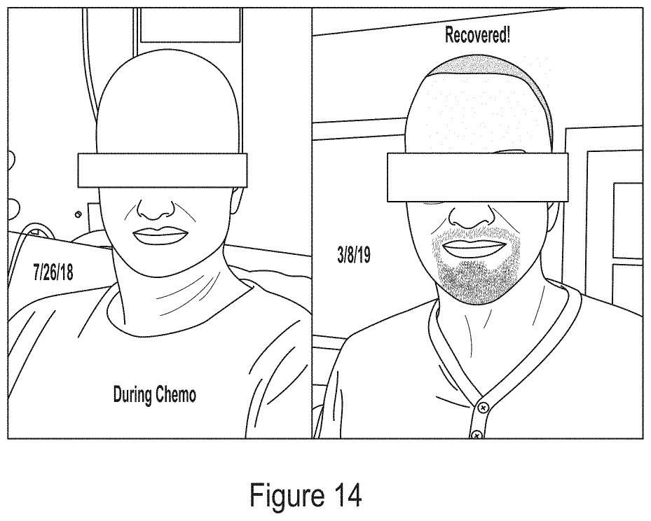

This report relates to treatment of a 69-year-old Australian male with the described composition (FIG. 14), as authorized by the Australian Therapeutic Administration (agency similar to FDA) through a Category A special access process. The patient was treated with the composition as described to address extreme fatigue after being treated for “Double-Hit” Non-Hodgkin's Lymphoma with R-hyper CVAD chemotherapy regimen.

### Example 2

### Effects of the Inventive Composition as Demonstrated in Horses Having Equine Lyme, Cushing's, or Laminitis

In this example, the inventive composition, as disclosed herein, was formulated as an intravenous (IV) drug therapy with the potential to correct underlying perfusion deficits and intracellular electrolyte and metabolic aberrancies associated with critical limb ischemia (CLI). The inventive composition contains acid-base chemistry components that cause an acidic bloodstream pH shift followed by an alkaline rebound, akin to aerobic exercise, but without incurring a coincidental oxygen debt. These components may also have the potential to elicit metabolic advantage. The inventive composition presents an increasing concentration of magnesium to the bloodstream, which can work with the acid-base chemistry to rebalance “levels” of intracellular ionic species. It can result in reduction of intracellular calcium and resolution of intracellular acidosis, which is favorable for downregulating inflammatory signaling and hypertension and restoring nitric oxide function in nerves and arteries while suppressing the immune inducible form. The inventive composition increases antioxidant and Bvitamin metabolic chain resources, which work along with the electrolytic correction to reduce metabolic oxidative stress. As a benefit of IV infusion, the inventive composition bypasses the gut to ensure elevated “time-synchronized” presentation of the species as desired. Thus, the inventive composition works to reverse common “stress biases” that prevent healing.

Equine Cushing's disease is a loss of dopaminergic control in the pituitary per intermedia, leading to adrenal gland dysfunction. There is an excess in the secretion of adrenocorticotropin hormone (ACTH), which causes downstream upregulation in the secretion of cortisol. Cushing's causes progressive dehabilitation, laminitis, delayed wound healing, chronic infections, parasitism, weight loss, diabetes mellitus, diabetes insipidus, excessive body fat, blindness, seizures, pseudolactaion, behavioral changes, and reproductive problems. Clinical signs include long and curly hair, excessive urination, thirst and sweating (McCue, Patrick M. The Veterinary Clinics: Equine Practice, vol. 18, no. 3, 2002, pp. 139-53). Finally, laminitis, a potential side effect of Cushing's, affects about 34% of horses and causes extreme pain with weight bearing in the hoof (Wylie, Claire E., et al. Veterinary Journal, vol. 193, no. 1, Elsevier Ltd, 2012, pp. 58-66). This can commonly lead to the need for euthanasia.

Lyme disease, or Borrelia Burgdorferi, is a bacterial threat that is delivered by a tick bite. Once inside an organism, Borrelia attacks cells by stealing pyruvate made in the first stage of glycolysis before it can contribute its full energetic yield to the cell, such as for the Kreb's cycle and Oxidative Phosphorylation. In measure to evade detection, Borrelia also elicits a fibrin response and steadily become wrapped in fibrin (Önder, Özlem, et al. Journal of Biological Chemistry, vol. 287, no. 20, 2012, pp. 16860-68). In this way, they effectively become cloaked in the body's own materials, becoming hidden from immune recognition. If not addressed completely and early, this cloaking helps Borrelia proliferate to degrade metabolic energy systems, cell-by-cell. As more cells become affected by this cycle and have their metabolism fail, the macroenvironment steadily becomes more acidic. This second hit of acidosis further favors the integrity of the cloaking because an alkaline environment would promote plasmin, which is capable of degrading fibrin. Thus, if Lyme is allowed to gain the upper hand, it can perpetuate its advantage through subsequent replication, cloaking, and degradation of energy systems to promote further acidification (Carroll, James A., et al. Infection and Immunity, vol. 68, no. 12, 2000, pp. 6677-84).

### 1.1. Methods

This example characterizes methods and results for the first-ever equine application of the inventive composition (also referred to as “RJX G2” in this study) in 3 horses. Studies were conducted in November 2018 at the Frost Ranch in Chester County, Pennsylvania through administration of the inventive composition to three horses. Subject 1 was a 34-year-old mare, Welsh Cross that was 739 pounds having a history of pre-diabetes, Laminitis with Cushing's disease, and Lyme disease. Subject 2 was a 13-year-old gelding, Welsh Cross that was 724 pounds having a history of Laminitis with Cushing's disease, and Lyme disease. Subject 3 was a 12-year-old mare, Welsh Cross that was 652 pounds and has a history of Lyme disease.

The inventive composition was provided in a two-vial system, each of which contains 100 mL of solution. A-Vial (USP Acid Shifting) is a proprietary acid with B vitamins, magnesium, and antioxidants. A secondary A-Vial-Buffered Acid Only, of which all ingredients were excluded from the solution except the acid and buffer components. B-Vial (USP) is a buffer solution. A-Vial products were refrigerated at 40° F. prior to use, while B-Vial products were stored at 70° F. 100 ml of A-Vial product was combined into a saline IV bag (either 1000 or 2000 mL), and then 100 ml of B-Vial product was combined into the IV bag. The IV bag was hung from an elevation point, 18″ above infusion point. A catheter was inserted into the jugular vein of the subject. Five minutes prior to treatment, venous blood samples were extracted from the patient for analysis (IDEXX Laboratories: hematology, chemistry, endocrinology, and serology) and blood gas analysis (acid/base status, oximetry, electrolytes, metabolites) (T=−5 min). Five minutes (T=0 min) later, the IV bag was connected to a catheter, and the drip was opened to begin infusion. The drip rate was adjusted to complete infusion by 45 minutes (T=45 min). Venous blood samples were extracted from the subject during treatment, 20 minutes (T=20 min) after the treatment began. Post-treatment venous blood samples were extracted after the treatment began for up to 120 minutes from time 0. Post-treatment samples were subjected to blood gas analysis (acid/base status, oximetry, electrolytes, and metabolites) All markers were taken at Dose 1 (day 1), Dose 4 (day 6), and Dose 5 (day 8). Dosing and variations in the inventive composition are described in Table 2.1-2.3. It should be noted that Subject 1's “pre-treatment” venous blood samples for (hematology, chemistry, endocrinology, and serology) were mistakenly sampled 60 minutes after treatment began. The results likely reflect post-dose changes in plasma volume, as large changes were observed for concentration-based markers (e.g., RBC, hematocrit).

TABLE 2.1
Subject 1 Dosing

| | DOSE 1 | DOSE 2 | DOSE 3 | DOSE 4 | DOSE 5 |
|---|---|---|---|---|---|
| | DAY 1 | DAY 2 | DAY 3 | DAY 6 | DAY 8 |
| 100 ml A-Vial of the | | | | | |
| inventive composition - | | | | | |
| ACID ONLY | | | | | |
| 100 ml A-Vial of the | X | X | X | X | X |
| inventive composition | | | | | |
| 100 ml B-Vial | X | X | X | X | X |
| USP Bicarb | | | | | |
| 1000 ml Saline | X | X | X | X | X |
| 2000 ml Saline | | | | | |

TABLE 2.2
Subject 2 Dosing

| | DOSE 1 | DOSE 2 | DOSE 3 | DOSE 4 | DOSE 5 |
|---|---|---|---|---|---|
| | DAY 1 | DAY 2 | DAY 3 | DAY 6 | DAY 8 |
| 100 ml A-Vial of the | X | | | | |
| inventive composition - | | | | | |
| ACID ONLY | | | | | |
| 100 ml A-Vial of the | | X | X | X | X |
| inventive composition | | | | | |
| 100 ml B-Vial | X | X | X | X | X |
| USP Bicarb | | | | | |
| 1000 ml Saline | X | X | X | X | X |
| 2000 ml Saline | | | | | |

TABLE 2.3
Subject 3 Dosing

| | DOSE 1 | DOSE 2 | DOSE 3 | DOSE 4 | DOSE 5 |
|---|---|---|---|---|---|
| | DAY 1 | DAY 2 | DAY 3 | DAY 6 | DAY 8 |
| 100 ml A-Vial of the | | | | | X |
| inventive composition - | | | | | |
| ACID ONLY | | | | | |
| 100 ml A-Vial of the | X | X | X | X | |
| inventive composition | | | | | |
| 100 ml B-Vial | X | X | X | X | X |
| USP Bicarb | | | | | |
| 1000 ml Saline | | X | X | X | X |
| 2000 ml Saline | X | | | | |

### 1.2. Results & Discussion

### Safety and Comfort Experience:

Horses were calm during administration and showed no signs of distress. For this work, infusion drip-rate was controlled to achieve a 45-minute infusion duration. The infusion time can likely be substantially reduced (˜15 min or less), but was extended here as a first in species precaution. Optimization of presentation rate is a subject of ongoing work.

### Blood Gas (Oxygen Status) and Acid-Base Response:

The exercise-like attribute of the inventive composition was assessed by measuring venous blood gases, including pH, bicarbonate (HCO₃⁻), pO₂ (partial pressure of oxygen), pCO₂ (partial pressure of carbon dioxide), and sO₂ (oxygen saturation of hemoglobin). Although arterial blood gases remain the gold standard for assessing the completeness of oxygen transfer from the lungs to the blood, venous blood gases were recorded for this work in the interest of patient comfort and safety (more risk and pain is associated with accessing high-pressure arterial structures deep under tissue versus low-pressure surface veins). At the same time, venous measurements indicate the completeness of oxygen delivery to the tissues, i.e., whether oxygen delivery met the needs or exceed them.

Tables 2.4-2.6 below present the time history of blood gas response as measured at T −5, 20, 50 min for Doses 1, 4, and 5 of the inventive composition in each of the subjects. Select samples were unavailable for subjects 1 and 3 (indicated as *). Although the response of all horses was materially similar, Subject 2 will be presented as primary example as all samples were successfully recorded.

TABLE 2.4
Subject 1 Response for Dose 1, 4, 5 of the inventive composition

| | | Day 1 Dose 1 | | | Day 6 Dose 4 | | | Day 8 Dose 5 | | |
|---|---|---|---|---|---|---|---|---|---|---|
| | | Time min | | | | | | | | |
| | | −5 | 20 | 50 | −5 | 20 | 50 | −5 | 20 | 50 |
| pH | — | 7.444 | * | * | 7.426 | 7.435 | 7.448 | 7.429 | 7.447 | 7.431 |
| cHCO3— | mmol/L | 34.1 | * | * | 30.1 | 31.1 | 31.3 | 30.1 | 29.9 | 29.6 |
| pCO2 | mmHg | 49.7 | * | * | 50.8 | 50.5 | 48.5 | 49.6 | 45.8 | 47.7 |
| pO2 | mmHg | 30 | * | * | 24 | 29 | 35 | 36 | 37 | 36 |
| sO2 | % | 59 | * | * | 48 | 59 | 71 | 73 | 76 | 73 |

TABLE 2.5
Subject 2 Response for Dose 1, 4, 5 (Note “acid-buffer” Dose 1) of the inventive composition

| | | ACID ONLY | | | | | | | | |
|---|---|---|---|---|---|---|---|---|---|---|
| | | Day 1 Dose 1 | | | Day 6 Dose 4 | | | Day 8 Dose 5 | | |
| | | Time min | | | | | | | | |
| | | −5 | 20 | 50 | −5 | 20 | 50 | −5 | 20 | 50 |
| pH | — | 7.392 | 7.437 | 7.431 | 7.350 | 7.416 | 7.394 | 7.453 | 7.426 | 7.417 |
| cHCO3— | mmol/L | 33.2 | 31.0 | 31.0 | 26.7 | 26.4 | 26.6 | 29.4 | 27.8 | 27.3 |
| pCO2 | mmHg | 54.5 | 45.9 | 46.5 | 57.3 | 43.2 | 47.7 | 44.3 | 44.5 | 45.1 |
| pO2 | mmHg | 30 | 34 | 35 | 24 | 39 | 31 | 37 | 39 | 33 |
| sO2 | % | 55 | 67 | 69 | 39 | 76 | 59 | 73 | 76 | 66 |

TABLE 2.6
Subject 3 Response for Dose 1, 4, 5 (Note “acid-buffer”
Dose 5) of the inventive composition

| | | | | | | | | ACID ONLY | | |
|---|---|---|---|---|---|---|---|---|---|---|
| | | Day 1 Dose 1 | | | Day 6 Dose 4 | | | Day 8 Dose 5 | | |
| | | Time min | | | | | | | | |
| | | −5 | 20 | 50 | −5 | 20 | 50 | −5 | 20 | 50 |
| pH | — | 7.455 | * | * | 7.437 | 7.392 | 7.428 | 7.423 | 7.412 | 7.375 |
| cHCO3— | mmol/L | 32.5 | * | * | 27.1 | 27.3 | 27.7 | 26.2 | 26.0 | 25.5 |
| pCO2 | mmHg | 46.2 | * | * | 42.6 | 49.9 | 45.0 | 42.4 | 43.6 | 49.9 |
| pO2 | mmHg | 29 | * | * | 32 | 29 | 34 | 34 | 31 | 20 |
| sO2 | % | 57 | * | * | 67 | 57 | 69 | 70 | 64 | 34 |

### Subject 2—Observed Response Following Dose 1, 4, and 5

### (I) Subject 2—Dose I Response (Buffered Acid Only):

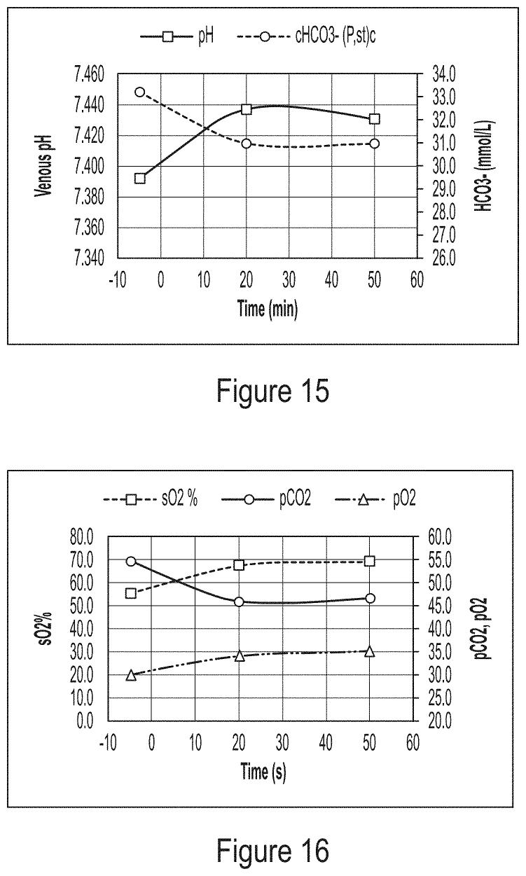

The Dose 1 response shown in FIG. 15, reveals venous pH rising towards alkaline from pre-dose levels during treatment. The pH then fell back slightly towards acidic post-dose. Although ACID ONLY solution would be expected to shift the bloodstream towards acidic, this was not observed at T=20. It is possible that the blood did indeed shift acidic before T=20, and this observation point occurred after the renal and respiratory compensation processes had already begun to control acid-base status.

Incoming values of venous HCO₃⁻ at Dose 1 were elevated from normal (33.2 mmol/L; normal is below 30.1 mmol/L), as is consistent with compensation during Cushing's disease. Thus HCO₃⁻“drags up” the bloodstream pH by buffering excess metabolic H⁺ (effectively binding with them to hide them from being measured as pH). During dosing, HCO₃⁻ was reduced by T=20 to 31 mmol/L. This could be explained by action of H⁺ to promote transport of HCO₃⁻ to the intracellular and/or renal extraction.

Concurrently as shown in FIG. 16, several changes in blood oxygen were observed. Venous sO₂ rose from 55% to 69% and pO₂ rose from 30 mmHg to 35 mmHg, while pCO₂ fell from 54.5% to 46.5%. While heart rate and respiration were not strictly measured, no elevation in labor or respiration was observed.

### (2) Subject 2—Dose 4 Response:

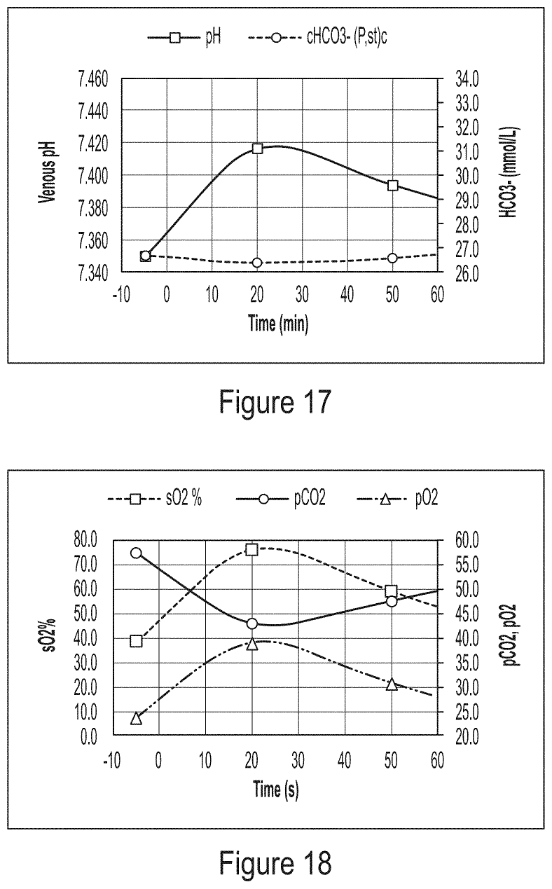

As shown in FIG. 17, the venous pH response was similar in many ways to the Dose 1 response; it rose towards alkaline during dosing and reduced back towards acidic post-dose. The pre-dose pH was slightly more acidic than that observed pre-Dose 1, which could represent an increase in metabolic H⁺ load, a reduction in HCO₃⁻, or a temporary adjustment of the renal “setpoint.” By Dose 4, large differences in the HCO₃⁻ status were apparent. Healthy normal levels of HCO₃⁻ (26.7 mmol/L) were measured as opposed to the high compensatory levels of HCO₃⁻ seen prior to Dose 1 (33.2 mmol/L). Finally, as shown in FIG. 18, the venous sO₂ and pO₂ were observed to rise during administration of the inventive composition, along with a reduction in pCO₂, consistent with the Dose 1 response.

### (3) Subject 2—Observed Response for Dose 5:

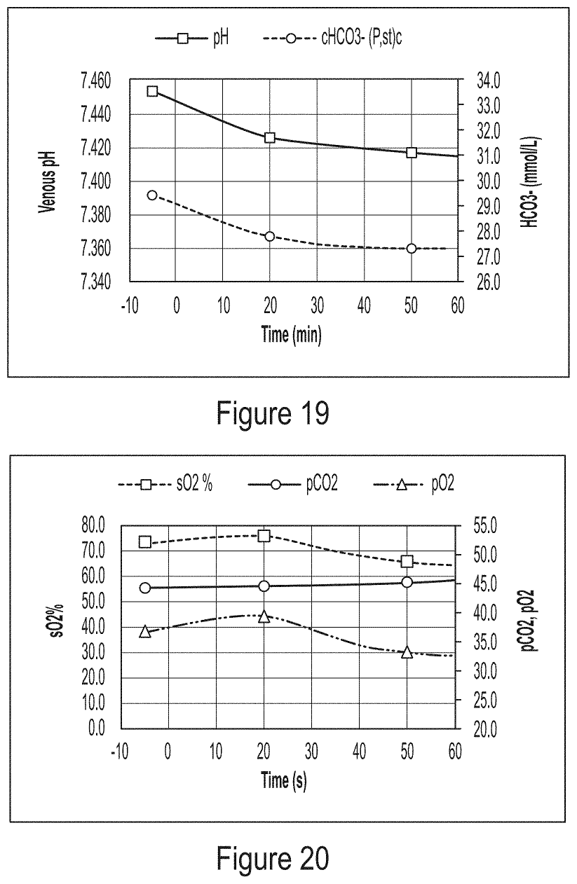

By Dose 5, substantial changes in the pH and oximetry response were observed. As shown in FIG. 19, the dose 5 pre-dose status was BOTH alkaline (pH=7.453) and uncompensated (HCO₃⁻=29.4 mmol/L). This suggests there could be Cushing's resolution. Regarding pH response, the Dose 5 pH was observed to drop towards acidic throughout the observation frame unlike the response for Dose 1 and 4. This possibly could be attributed to the alkaline starting bias and lack of compensatory HCO₃⁻, such that the acidic infusion could impart a sustained response. Instead of rising or remaining unchanged, bloodstream HCO₃⁻ reduced throughout the observation period, consistent with flow into the intracellular and/or enhanced renal extraction. This would also be counter to a Cushing's compensation, where bloodstream retention of HCO₃⁻ would be expected.

As shown in FIG. 20, the Dose 5 oximetry was affected. First, the pre-dose sO₂ was observed at 73% with a pO₂ of 37 mmHg, potentially indicating a durable elevation of oxygen servicing between doses. During administration, sO₂ and pO₂ were observed to rise further while pCO₂ remained largely unchanged.

### Overview of pH and Oximetry Takeaways for all Subjects:

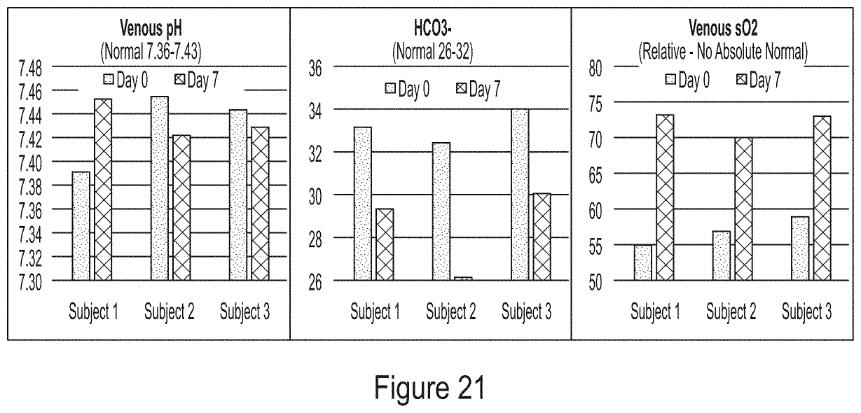

Although there were subtle differences in response, many similarities could be observed between the presented Subject 2 response and those observed for Subjects 1 and 3, as shown in FIG. 21. For instance, all subjects showed a “conditioning effect” over the 4 doses (as measured Day 7 before dose 5) such that an enhanced homeostasis was observed regarding acid/base status and oxygen servicing. After 4 doses, all subjects presented as biased towards alkaline (pH >7.42) without bicarbonate compensation (HCO₃⁻ normal at 30.1 mmol/L or below). These results suggest a more complete metabolic chain allowing a reduction in acid load and/or an improvement in a renal acid management function. The observed increases in venous sO2 and pO2 could indicate an improvement in oxygen delivery capacity, which would favor a more aerobically complete metabolic chain. These attributes potentially indicated that the metabolic source of Cushing's dysfunction is affectable.

Electrolyte, Hb, Glu, and Lac Response from Dose 4 and 5:

In addition to the acid-base and oximetry signals, subjects were monitored for select blood electrolytes, hemoglobin (Hb), glucose (Glu), and lactate (Lac). Dose 1 was not measured for broad electrolyte response as the on-site instrument was out of calibration for these parameters. Calibration was achieved in time to support the Dose 4 and 5 records, and are presented in Table 2.7. As before, Subject 2's data is presented as the narrative example for each of the signals under study.

TABLE 2.7
Subject 2 Response for Dose 4 and 5 of the inventive composition

| | | Day 6 Dose 4 | | | Day 8 Dose 5 | | |
|---|---|---|---|---|---|---|---|
| | | Time min | | | | | |
| | | −5 | 20 | 50 | −5 | 20 | 50 |
| cK⁺ | mmol/L | 4.6 | 4.2 | 2.9 | 4.4 | 4.0 | 3.7 |
| cNa⁺ | mmol/L | 140.0 | 138.0 | 144.0 | 137.0 | 137.0 | 138.0 |
| cCa²⁺ | mmol/L | 1.7 | 1.7 | 1.6 | 1.7 | 1.6 | 1.6 |
| cCl⁻ | mmol/L | 103.0 | 103.0 | 105.0 | 100.0 | 101.0 | 101.0 |
| ctHb | g/dL | 14.8 | 11.9 | 13.2 | 13.2 | 11.2 | 11.0 |
| cGlu | mg/dL | 105.0 | 115.0 | 99.0 | 91.0 | 83.0 | 89.0 |
| cLac | mmol/L | 1.3 | 0.7 | 0.3 | 0.4 | 0.5 | 0.5 |

### Subject 2's Hb, Glu, Lac, and Electrolyte Response:

Over the observation timeframe, the Hb final measurement is reduced from its initial value with evidence of rebound during the observation period in Dose 4. Subject 2's Dose 4 response shows Hb reducing in concentration from 14.8 g/dL to 11.9 g/dL (25% dilution) in 25 minutes before rebounding to 13.2 g/dL. A Hb rebound is seen in subject 2, which cannot be interpreted as hemolysis, as a rebound on this timescale would be impossible. The change in Hb concentration is most likely attributed to a change in blood volume from vasodilation. In this case, plasma would be drawn from the intracellular (for instance, from inflamed cells) into the blood to support the vessel volume created in the dilated state (Brocker, Chad, et al. Biomolecular Concepts, vol. 3, no. 4, 2012, pp. 345-64). Such exchange is required during exercise-like stimulus to maintain vascular pressure, under conditions of vasodilation, where vascular volume increases. Thus, vasodilation was seemingly confirmed, along with a potential to reduce inflammation.

Over the observation timeframe, Glu was perturbed (up and down) from its start value during Dose 4, while showing evidence of rebound during the observation period in Dose 5. While the reduction could be attributable to increased blood volume, elevations in Glu concentration cannot. This observed glucose elevation is consistent with perturbation of glucose exchange, such as happens during exercise (Richter, E. A., et al. Skeletal Muscle Metabolism in Exercise and Diabetes. 1998). Such an effect could be instrumental in the conditioning of parasympathetic controls, such as pituitary, adrenal, thyroid, and pancreatic function.

Lactate was reduced to a level below typical resting values during Dose 4 (<1 mmol/L; normal equine resting lactate is 1-2 mmol/L). Lactate levels reduced from “normal” levels (1.3 mmol/L) to a low value (0.3 mmol/L) in 55 minutes. In addition, the pre-Dose 5 measurement lactate was below normal (0.4 mmol/L) and remained below over the 55 minutes of measurements. Lactate represents oxygen debt from anerobic processes that lack oxygen, reduction of lactate is consistent with an aerobically complete metabolic chain. Lactate remained below normal levels the whole time it was in Dose 5, potentially implying a sustained effect between doses. The reductions in lactate were observed despite the alkaline swing, which should release stored lactate from muscles (Robergs, Robert A., et al. American Journal of Physiology. Regulatory, Integrative and Comparative Physiology, vol. 287, no. 3, 2004, pp. R502-16). Thus, the reductions in lactate could be inclusive of both endogenous and stored components.

During the infusion process, an electrolyte exchange was observed. Blood levels of potassium (K⁺) and sodium (Na⁺) were observed to drop during treatment. Specifically, K⁺ decreased to 37% over 55 minutes of observation (e.g., Dose 4: 4.6 mmol/L→2.9 mmol/L). Modest changes in the other electrolytes were also observed during the treatment period. The electrolyte exchanges observed in subject 2 were materially similar to those observed in the other subjects, suggesting that the exchange process may be repeatable across subjects. Effects regarding calcium are further discussed in the review of blood “chemistry,” as net reductions in bloodstream calcium were noted in subsequent blood draws following the week of dosing. Bloodstream calcium (Ca²) levels were also observed to drop for all subjects.

### Hematology, Chemistry, Endocrinology, and Serology:

Blood draws were also taken from each subject to assess Hematology, Chemistry, Endocrinology, and Serology differences between Day 1 pre-Dose 1 and Day 8 following Dose 4 but preceding Dose 5. The measured quantities are summarized in Table 2.8 and discussed further below.

TABLE 2.8
Hematology, Chemistry, Endocrinology, Serology Encompassing 4 doses in 7 days in all 3 horses.

| | | Patient | | | | | | |
|---|---|---|---|---|---|---|---|---|
| | | Subject 1 | | Subject 2 | | Subject 3 | | |
| | | Day 1 | Day 8 | Day 1 | Day 8 | Day 1 | Day 8 | |
| | | Pre- | Post- | Pre- | Pre- | Pre- | Pre- | Reference |
| Test | Notes | sample | sample | sample | sample | sample | sample | Values |
| HEMATOLOGY | | | | | | | | |
| WBC | | 6.1 | 4.4 | 7.8 | 7.2 | 8.9 | 9.0 | 4.3-11.4 |
| | | | | | | | | K/uL |
| Neutrophils | | 3.251 | 2.275 | 3.962 | 3.168 | 3.382 | 4.329 | 2.46-7.23 |
| | | | | | | | | K/uL |
| Lymphocytes | | 2.513 | 1.76 | 3.143 | 2.938 | 4.806 | 3.933 | 1.45-5 |
| | | | | | | | | K/uL |
| Monocytes | | 0.232 | 0.198 | 0.296 | 0.36 | 0.712 | 0.279 | 0-0.6 |
| | | | | | | | | K/uL |
| Eosinophils | | 0.092 | 0.158 | 0.39 | 0.706 | 0 | 0.441 | 0-0.7 |
| | | | | | | | | K/uL |
| Basophils | al | 0.012 | 0.009 | 0.008 | 0.029 | 0 | 0.018 | 0-0.1 |
| | | | | | | | | K/uL |
| Platelets | | 106 | 141 | 129 | 192 | 148* | 198 | 70-250 |
| | | | | | | | | K/uL |
| Fibrinogen | | 116 | 127 | 129 | 168 | 131 | 147 | 135-249 |
| | | | | | | | | mg/uL |
| CHEMISTRY | | | | | | | | |
| Creatinine | | 0.7 | 0.9 | 1.2 | 1.4 | 1.2 | 1.4 | 0.8-1.8 |
| | | | | | | | | mg/uL |
| BUN:Creatinine | | 30.0 | 23.3 | 10.8 | 10.0 | 11.7 | 7.9 | |
| Ratio | | | | | | | | |
| Calcium | | 13.0 | 11.6 | 12.4 | 11.6 | 12.2 | 11.6 | 10.2-12.8 |
| | | | | | | | | mg/uL |
| Sodium | | 136 | 136 | 136 | 137 | 136 | 137 | 132-141 |
| | | | | | | | | mmol/L |
| Potassium | | 4.8 | 4.1 | 5.2 | 3.7 | 5.4 | 3.9 | 2.5-5.2 |
| | | | | | | | | mmol/L |
| Creatine Kinase | | 334 | 221 | 271 | 210 | 349 | 259 | 130-497 |
| | | | | | | | | U/L |
| ENDOCRINOLOGY | | | | | | | | |
| Total T4 | | 1.5 | 1.8 | 2.5 | 3 | 1.7 | 2.6 | 1-3.8 |
| | | | | | | | | ug/dL |
| Equine Endogenous | a3 | 26 | 24 | 19 | 18 | 26 | 16 | 9-35 |
| | | | | | | | | pg/mL |
| ACTH | | | | | | | | |
| SEROLOGY | | | | | | | | |
| Lyme Antibody by | a4 | Positive | Positive | Positive | Positive | Positive | Positive | |
| IFA | | @1:3200 | @1:800 | @1:800 | @1:200 | @1:800 | @1:200 | |
| Lyme OspA | | 123 Negative | 129 Negative | 197 Negative | 242 Negative | 225 Negative | 201 Negative | |
| Lyme OspC | | 73 Negative | 77 Negative | 238 Negative | 272 Negative | 79 Negative | 69 Negative | |
| Lyme OspF | b2 | 3000 Positive | 3936 Positive | 318 Negative | 390 Negative | 464 Negative | 498 Negative | |
| Ehrlichia canis | c2 | Negative | Postitive | Negative | Negative | Negative | Negative | |
| Antibody by IFA | | | @1:100 | | | | | |

Creatinine rose in all subjects and the ratio of blood urea nitrogen (BUN): creatinine decreased for all subjects, consistent with increased flow through the kidneys. Creatinine measurements. Consistent with an increase in muscle mass and improved capacity to store adenosine triphosphate (ATP) in muscle as Phosphocreatine.

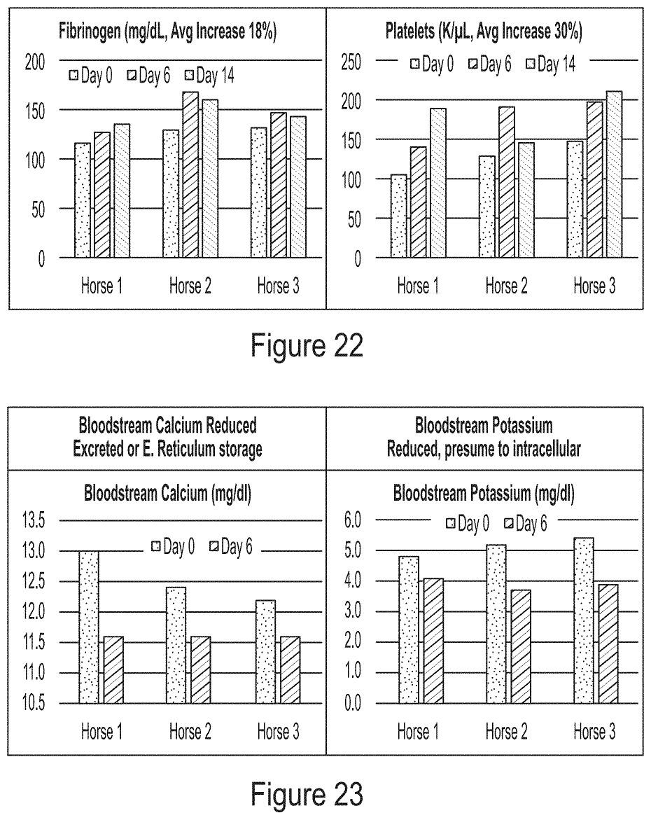

Platelet counts and fibrinogen were increased for all subjects (Table 2.8 and FIG. 22), consistent with control over clotting cascade and reduced consumption of clotting products. This could also be consistent with increased production of platelets in bone marrow upon resolution of Thrombocytopenia and increased presentation of fibrinogen through enhanced liver function.

Bloodstream Ca²⁺ and K⁺ were observed to drop for all subjects (FIG. 23), consistent with intracellular uptake of K⁺ via Na⁺/K⁺ ATPase, and renal extraction of Ca²⁺, so as to reduce bloodstream presentation. Reductions in Ca²⁺ and increases in K⁺ could have the potential to reduce chemiosmotic gradient dependence on Ca²⁺ so as to restore electron chain transport function, reduce metabolic reactive oxygen species (ROS), promote alkaline conditions, and increase basal metabolic rate. These factors could work with elevated Mg⁺² to favor restoration of peroxisome functions: metabolism of long-chain fatty acids, myelin maintenance for nerve function, and catalase servicing for antioxidant action against peroxide. Additionally, lower Ca²⁺ could reduce caveolae bound Caveolin to allow endothelial nitric oxide synthase (eNOS) to translocate from the Golgi back to functional locations in the membrane caveolae. Lower intracellular calcium could also signal more “healing” M2 phenotype presentation for macrophages, microglia, and osteoblasts, among others. Increased K⁺ could act to enhance muscle function and nerve transmission, reduce cramping of muscles, and provide other benefits. These pathological details are presented as they relate to various symptomatic improvements that have been observed in past compassionate use of the inventive composition by FDA, and which are related to K⁺ and Ca²⁺.

Creatine kinase dropped in all 3 subjects (Table 2.8); this is consistent with a potential increase of consumption of creatine kinase in enzymatic action to promote storage of ATP with creatine as Phosphocreatine to enhanced stored energy in muscles. Alternately, the reduction in blood plasma creatine kinase can indicate a reduction in the ongoing rate of tissue damage, such as in myocardial infarction (heart attack), rhabdomyolysis (severe muscle breakdown), muscular dystrophy, autoimmune myositis, and acute kidney injury, so as to minimize presentation of damaged tissue contents to the bloodstream.

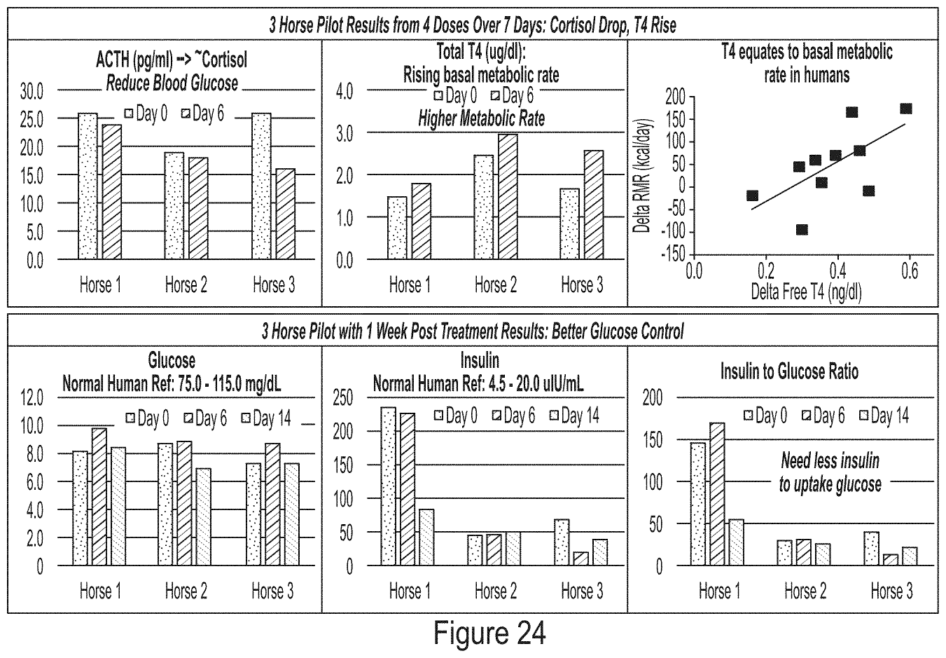

Total T4 (thyroxine) was observed to rise for all subjects, potentially indicating improved thyroid function through increased production of thyroxine. The alteration in T4 is commonly downregulated in autoimmune disorders (such as in Cushing's), leading to a reduction in metabolism. When resolved, it is associated with an increase in synthesis of the Na⁺/K⁺ ATPases, glucose absorption, glycogenolysis, gluconeogenesis, lipolysis, protein synthesis, net catabolic degradation, cardiac beta-1 receptors for enhanced sympathetic nervous control, and basal metabolic rate (Johannsen, Darcy L., et al. Effect of Short-Term Thyroxine Administration on Energy Metabolism and Mitochondrial Efficiency in Humans. Vol. 7, no. 7, 2012). Equine endogenous adrenocorticotropic hormone (ACTH) was observed to fall for all subjects, which is consistent with a reduction in cortisol levels (FIG. 24). A reduction in cortisol levels promotes calming and anti-anxiety effects, as well as promoting resolution of Cushing's disease (Elzinga, Bernet M., et al. Neuropsychopharmacology, vol. 28, no. 9, 2003, pp. 1656-65). Elevated cortisol is commonly associated with heightened stress and anxiety, such as in post-traumatic stress disorder. The increase in T4 and the reduction in ACTH suggests an improvement in energy system control and glucose control, which are central in hypothyroid dysfunction. As shown in FIG. 11, an improvement in the relationship between insulin and glucose coincided with the alterations in thyroid hormones. Specifically, less insulin was required to support glucose uptake. One could interpret that after 4 doses, the metabolic chain is altered to make less metabolic acid, allowing alkalization to be achieved intracellularly to promote the subsequent improvement in insulin sensitivity.

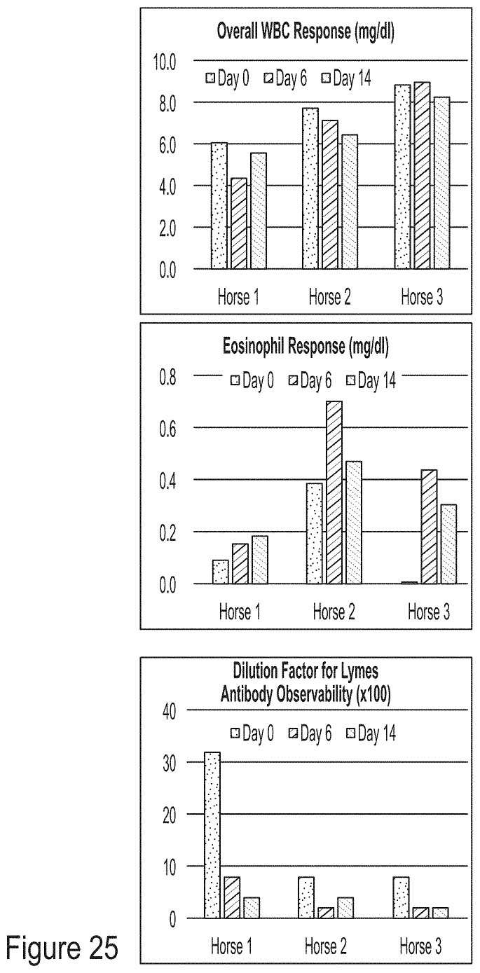

White blood cell (WBC) and neutrophil counts were observed to drop for 2 of 3 subjects (Subject 1 and 2), consistent with alleviation of inflammation response (FIG. 25). Despite the observed reduction in overall WBC's, a substantial increase of Eosinophils was observed for all subjects. In this case, the inventive composition may have promoted an anti-inflammatory alkaline environment, which would promote clearance of inflammatory macrophages. Despite the observed reduction in overall WBC's, a substantial increase of Eosinophils was observed for all subjects (FIG. 25).

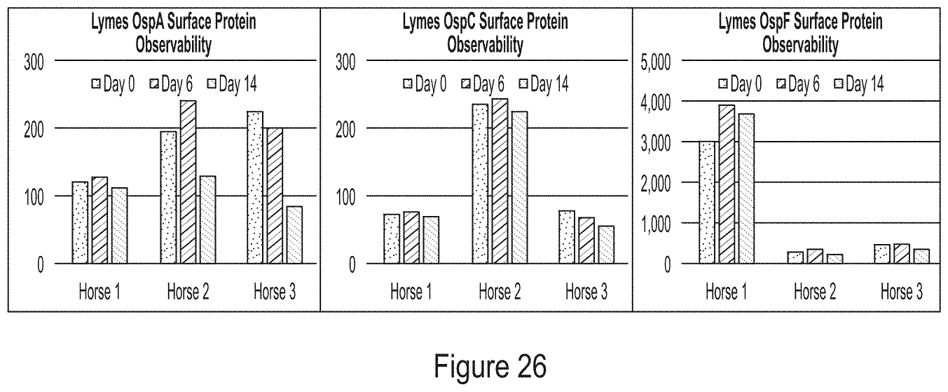

Lyme antibodies, as measured by observability at dilution ratio (FIG. 26), were shown to reduce by a factor of 4 in all subjects after one week of treatment. In this measure, a smaller divisor means that a blood sample can only be diluted a small amount before the antibody becomes undetectable. Lyme surface proteins were also observed to increase after one week in most cases for all subjects and then fall when observed after a week without treatment (FIG. 26). This would be consistent with a successful antibody response as surface proteins represent the exposed shell of a defeated pathogen, which rises after successful immune response and are subsequently cleared from the system.

### 1.3. Conclusions

The inventive composition is able to provide an acute exercise-like stimulus, whereby the bloodstream pH is shifted acidic to cause compensatory renal and respiratory processes to respond. All the while, the pH gradient (H⁺ gradient) from the blood to the cell is disturbed to facilitate a cascade of exchange between the bloodstream and intracellular for ionic currencies, including H⁺, HCO₃⁻, Ca²⁺, K⁺, Na⁺, Mg²⁺, and Cl⁻. At the same time, pH also affects hemoglobin affinity for O₂, activity of enzymes, insulin pairing, and glucose uptake, and vasodilation.

The uncompensated (HCO₃⁻=30.1 mmol/L or below) and alkaline biased (pH >7.42) acid-base status observed for all subjects may suggest a more complete metabolic chain to reduce acid load and/or an improvement in renal acid management function. The observed increases in venous sO₂ and pO₂ could indicate an improvement in oxygen delivery capacity, which would favor a more aerobically complete metabolic chain. This could be an indication that the metabolic chain impairments in Cushing's are correcting so as to make less metabolic acid (H⁺), and correspondingly require less bloodstream HCO₃⁻ to control pH (Tritos, et al. Clinical Neuroendocrinology, 1st ed., vol. 124). While one can appreciate that the pH stimulus of the inventive composition could re-set the electrolytic status within the cell, other ingredients in the inventive composition, including B-vitamins, Mg⁺², and antioxidants should also be expected to improve the metabolic chain as suggested by the results. Of course, the simultaneous drop in pH indicates that the renal setpoint is also adjusting. These attributes potentially indicate that the metabolic source of Cushing's dysfunction is affectable in the horses.

When “extra” O₂ makes it back to the lungs (high venous sO₂), it is suggested that the heart rate, respiratory rate, number of red blood cells (RBC's), and hemoglobin affinity for oxygen in each RBC is operating in excess relative to levels required for tissue survival. Alternately when sO₂ and pO₂ are low, it may be interpreted that some tissues are under-served with oxygen as there was little “left over”. Thus, the enhanced servicing of oxygen to tissues may be the result of enhanced hemoglobin affinity for O₂. These enhancements are consistent with the literature under alkaline conditions and conditions of elevated HCO₃⁻ in RBC's as both of these mechanisms reduce the competition of H⁺ for hemoglobin sites, leaving more sites available for oxygen (Oellermann, M., et al. Journal of Experimental Biology, vol. 217, no. 9, 2014, pp. 1430-36). These results suggest the inventive composition safely shifts the bloodstream pH and stimulate a series of compensatory effects, including potentially enhancing blood oxygen status and signaling the need for new RBC's. Similar events are observed in the context of athletic training.

Blood K⁺ and Na⁺ dropped during treatment, which could be attributed to increased plasma volume, renal extraction, or flow to the intracellular. K⁺ which if presented to the intracellular might be expected to enhance muscle function and nerve transmission, reduce cramping of muscles, along with other benefits. Flow of K⁺ to the intracellular might, if ATP yield were improved, increase action of the Na⁺/K⁺ ATPase. A flow of H⁺ into the cell, such as from a pH shift, might be expected to elevate the Chemiosmotic gradient to promote such increases in ATP. Ca dropping is consistent with intracellular uptake of K⁺ via Na⁺/K⁺ ATPase, and renal extraction of Ca²⁺, so as to reduce bloodstream presentation. Reductions in Ca²⁺ and increases in K⁺ could have the potential to reduce chemiosmotic gradient depending on Ca²⁺. These alterations could restore the electron transport chain function, reduce metabolic reactive oxygen species, promote alkaline conditions, and increase basal metabolic rate. These factors could work with elevated Mg²⁺ to favor restoration of peroxisome functions: metabolism of long-chain fatty acids, myelin maintenance for nerve function, and catalase servicing for antioxidant action against peroxide. Additionally, lower Ca²⁺ could reduce caveolae bound caveolin to allow eNOS to translocate from the Golgi back to functional locations in the membrane caveolae. Lower intracellular calcium could also signal more “healing” M2 macrophages, microglia, osteoblasts, and others (Xu, Rende, et al. Arteriosclerosis, Thrombosis, and Vascular Biology, vol. 37, no. 2, 2017, pp. 226-36).

The white blood cells dropped in two of the horses, but eosinophils rose. Given that this species of WBC, eosinophils are especially suited to fighting bacterial threats, such as Lyme disease, it is proposed that eosinophils were more readily recruited after a broad immune quiescing was achieved. Thus, alkaline conditions may have quiesced the broad WBC response to allow local sites under bacterial threat to have their signals for help be heard. Additionally, the alkaline conditions may have favored action of plasmin, to compromise fibrin cloaking and render Borellia visible for Eosinophil recruitment. A similar effect was observed in a compassionate care patient, who was treated with the inventive composition for post-chemotherapy fatigue. In studying this case, it was recognized that alkaline conditions reduce overall macrophage counts and might also alter the ratio between pro-inflammatory (e.g., M1, CD16⁺) and anti-inflammatory phenotypes (e.g., M2, CD16⁻). In the literature, it has been observed that such WBC alterations can be beneficial to outcomes in cancer patients (Imtiaz, Fauzia, et al. International Archives of Medicine, vol. 5, no. 1, BioMed Central Ltd, 2012, p. 2). Such alterations are also relevant in various auto-immune disorders, as in Lyme disease.

Collectively the Lyme results suggest a progression towards resolution. At a follow-up check 3 months post-treatment, 2 of 3 subjects remained Lyme-free. In a future study, it is proposed to re-treat the relapsing for a longer period to see if the condition can be durably cleared. Lyme disease had not been studied in other patients prior to the equine project, but now stands as a key focus for future study.

Laminitis, though not presented mechanistically above, proved to be in remission in the horses allowing them to go out to pasture. In conclusion, the inventive composition was demonstrated to be safe for use in equine. The study additionally presented potential evidence of efficacy for treating Cushing's disease, Lyme's disease, and Laminitis. Additionally, demonstrating enhanced tissue oxygenation, immune quiescing with enhanced selective WBC response, improved thyroid function, reductions in cortisol levels, restoration of clotting function while maintaining clotting selectivity, heightened metabolism, and electrolytic correction. Further validation of these observations is the subject of ongoing work.

1. A method of preventing, alleviating, or treating a hypoxia-related disease or condition, comprising administering an effective amount of a composition to a subject in need thereof to improve oxygen transport and thereby elevate blood oxygen levels, wherein the composition comprises:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3000 mmol/L when administered to a subject, and
wherein the selection of the pharmaceutical grade acid and the pharmaceutical grade pH buffering agent is effective to provide a buffer solution pH of between 4.0 and 7.7.

2. The method of claim 1, wherein the hypoxia-related disease or condition is cancer, angiogenesis, or an angiogenesis-related disorder.

3. The method of claim 1, wherein the cancer is a tumor or a solid tumor.

4. The method of claim 1, wherein the cancer is selected from the group consisting of breast cancer, pancreatic cancer, ovarian cancer, colon cancer, lung cancer, non-small cell lung cancer, in situ carcinoma (ISC), squamous cell carcinoma (SCC), thyroid cancer, cervical cancer, uterine cancer, prostate cancer, testicular cancer, brain cancer, bladder cancer, stomach cancer, hepatoma, melanoma, glioma, retinoblastoma, mesothelioma, myeloma, lymphoma, and leukemia.

5. The method of claim 1, wherein the composition increases intracellular HCO₃⁻ level and thereby promotes hemoglobin affinity for oxygen.

6. The method of claim 1, wherein the subject suffers a blood electrolyte imbalance.

7. The method of claim 4, wherein the blood electrolyte imbalance is a result of excess acid or bicarbonate.

8. The method of claim 1, wherein the method comprises elevating pO₂ level in the venous blood in the subject.

9. A method of treating a subject suffering from a condition characterized by elevated serum calcium, comprising administering an effective amount of a composition to the subject to reduce blood calcium levels, wherein the composition comprises:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3000 mmol/L when administered to a subject, and
wherein the selection of the pharmaceutical grade acid and the pharmaceutical grade pH buffering agent is effective to provide a buffer solution pH of between 4.0 and 7.7.

10. A method of restoring tumor suppressor protein p53 function in a subject, comprising administering an effective amount of a composition to the subject, wherein the composition comprises:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and
wherein the selection of the pharmaceutical grade acid and the pharmaceutical grade pH buffering agent is effective to provide a buffer solution pH of between 4.0 and 7.7.

11. A method of suppressing tumor aggression in a subject having a cancer while restoring angiogenesis in healthy tissue of the subject, comprising administering an effective amount of a composition to the subject to increase eNOS and suppress iNOS, wherein the composition comprises:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and
wherein the selection of the pharmaceutical grade acid and the pharmaceutical grade pH buffering agent is effective to provide a buffer solution pH of between 4.0 and 7.7.

12. A method of treating a subject having a cancer and suffering from elevated blood glucose related to the cancer, comprising administering an effective amount of a composition to the subject to improve pituitary, thyroid and renal function, thereby reducing blood glucose levels, wherein the composition comprises:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and
wherein the selection of the pharmaceutical grade acid and the pharmaceutical grade pH buffering agent is effective to provide a buffer solution pH of between 4.0 and 7.7.

13. The method of claim 12, wherein the composition reduces cortisol levels, thereby reducing circulating glucose by relieving mitochondrial stress and endoplasmic reticulum stress.

14. A method of inhibiting poly ADP ribose polymerase (PARP), comprising administering to a subject in need thereof an effective amount of a composition, wherein the composition comprises:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and
wherein the selection of the pharmaceutical grade acid and the pharmaceutical grade pH buffering agent is effective to provide a buffer solution pH of between 4.0 and 7.7.

15. A method of restoring a disturbed bone marrow microenvironment, comprising administering an effective amount of a composition to a subject in need thereof, the method comprising:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and
wherein the selection of the pharmaceutical grade acid and the pharmaceutical grade pH buffering agent is effective to provide a buffer solution pH of between 4.0 and 7.7.

16. A method of promoting apoptosis in cancer, comprising administering an effective amount of a composition to a subject in need thereof, thereby eliciting a temporarily elevated acidic pH in the bloodstream to further decreasing intracellular pH which results in acidic stress and apoptosis in cancer cells, wherein the composition comprises:
at least one pharmaceutical grade acid and at least one pharmaceutical grade pH buffering agent in a sterile aqueous solution,
wherein the concentration of the pharmaceutical grade acid and the pharmaceutical grade pH buffering agent in the buffer solution is sufficient to provide a total titratable acid content between 60 mmol/L and 3,000 mmol/L when administered to a subject, and
wherein the selection of the pharmaceutical grade acid and the pharmaceutical grade pH buffering agent is effective to provide a buffer solution pH of between 4.0 and 7.7.

17. The method of claim 1, wherein the subject is a human or a veterinary subject.

18. The method of claim 1, wherein the composition is delivered by intravenous, intramuscular, or parenteral administration, oral administration, otic administration, topical administration, inhalation administration, transmucosal administration and transdermal administration.

19. (canceled)

20. (canceled)

21. (canceled)

22. (canceled)

23. (canceled)

24. (canceled)

25. (canceled)

26. (canceled)

27. (canceled)

28. (canceled)

29. The method of claim 1, wherein the pharmaceutical grade acid is a physiologically acceptable acid.

30. The method of claim 1, wherein the pharmaceutical grade acid is hydrochloric acid, ascorbic acid, acetic acid, or a combination thereof.

31. The method of claim 1, wherein the at least one pH buffering agent is a physiologically acceptable buffer.

32. The method of claim 1, wherein the at least one pH buffering agent is sodium bicarbonate, a phosphate buffer, sodium hydroxide, an organic acid, an organic amine, ammonia, a citrate buffer, a synthetic buffer creating specific alkaline conditions or a combination thereof.

33. The method of claim 32, wherein the synthetic buffer is tris-hydroxymethyl aminomethane.

34. The method of claim 1, wherein the composition further comprises one or more ingredients selected from the group consisting of vitamins, salts, acids, amino acids or salts thereof, and stabilized oxidative species.

35. The method of claim 1, wherein the composition further comprises ascorbic acid.

36. The method of claim 1, wherein the composition further comprises dehydroascorbic acid.

37. The method of claim 1, wherein the composition further comprises other recognized antioxidant defense compounds including nonenzymatic compounds such as tocopherol (aTCP), coenzyme Q10 (Q), cytochrome c (C) and glutathione (GSH) and enzymatic components including manganese superoxide dismutase (MnSOD), catalase (Cat), glutathione peroxidase (GPX), phospholipid hydroperoxide glutathione peroxidase (PGPX), glutathione reductase (GR); peroxiredoxins (PRX3/5), glutaredoxin (GRX2), thioredoxin (TRX2) and thioredoxin reductase (TRXR2).

38. (canceled)

39. (canceled)

40. (canceled)

41. (canceled)

42. The method of claim 1, wherein the composition comprises pharmaceutical grade of:
900±90 mg of L-Ascorbic Acid;
63.33±6.33 mg Thiamine HCl;
808±80.8 mg of Magnesium Sulfate;
1.93±0.193 mg of Cyanocobalamin;
119±11.9 mg of Niacinamide;
119±11.9 mg of Pyridoxine HCl;
2.53±0.253 mg of Riboflavin 5′Phosphate;
2.93±0.293 mg of Calcium D-Pantothenate;
840±84 mg of Sodium Bicarbonate;
4.5±0.45 mM of HCl; and
water in an amount to obtain a final composition volume of 20 mL.

43. (canceled)

44. (canceled)

45. (canceled)
